SEQUENCE LISTING (55 sequences)

SEQ ID NO: 1 - RNA, 20 nt, synthetic construct
  source 1..20: mol_type = other RNA
cgcagccatgtgccgcaccc

SEQ ID NO: 2 - RNA, 20 nt, synthetic construct
  source 1..20: mol_type = other RNA
cctgagtctccacggcagtg

SEQ ID NO: 3 - RNA, 20 nt, synthetic construct
  source 1..20: mol_type = other RNA
caaagagttcaagacacgtc

SEQ ID NO: 4 - RNA, 20 nt, synthetic construct
  source 1..20: mol_type = other RNA
cattttgtctaactttggag

SEQ ID NO: 5 - RNA, 20 nt, synthetic construct
  source 1..20: mol_type = other RNA
tcattccagattccagatgt

SEQ ID NO: 6 - RNA, 20 nt, synthetic construct
  source 1..20: mol_type = other RNA
cgcccagccgcaaacagccg

SEQ ID NO: 7 - RNA, 20 nt, synthetic construct
  source 1..20: mol_type = other RNA
aggcagtggccacaagagcg

SEQ ID NO: 8 - RNA, 20 nt, synthetic construct
  source 1..20: mol_type = other RNA
agactctgctgctgtttgtg

SEQ ID NO: 9 - RNA, 20 nt, synthetic construct
  source 1..20: mol_type = other RNA
tcttgcagaaatgtccaatc

SEQ ID NO: 10 - RNA, 20 nt, synthetic construct
  source 1..20: mol_type = other RNA
tccaggcatgcagatcccac

SEQ ID NO: 11 - RNA, 20 nt, synthetic construct
  source 1..20: mol_type = other RNA
ggctgaacaagtccttcctc

SEQ ID NO: 12 - RNA, 20 nt, synthetic construct
  source 1..20: mol_type = other RNA
catggatgatggccaagtgc

SEQ ID NO: 13 - RNA, 20 nt, synthetic construct
  source 1..20: mol_type = other RNA
gcctgaacacaaggcatggc

SEQ ID NO: 14 - RNA, 20 nt, synthetic construct
  source 1..20: mol_type = other RNA
tccggcgacgcttgtccact

SEQ ID NO: 15 - RNA, 20 nt, synthetic construct
  source 1..20: mol_type = other RNA
taggatggtagcacacaacc

SEQ ID NO: 16 - DNA, 2017 bp, synthetic construct
  source 1..2017: mol_type = other DNA
gcggccgccgatcaaagaaagccctgtgtgcctcccagacccaccacccccgtgcaaactctctcaacacgacaccgctgcgtgactttcctgctgttactagaggtcggcaggtacttttccctaggaaactgggtgtgcacgtctcatccccgtgccaattctcttgccgcttttccttccacacgcggcgtcctctgggcggagggtgcagctccgcgtgctccagctgccgcgaaagttgcacccggcccagtctggctgcgggccccgcctccgccttctcagcgcccagccccaccctccgattggtcagcagtgcccagccccgccccgctcctccgaggggatttaaagcagcggccggtcagtctgggcgcttgcaggctgctaaacccaaccgcagttgactagcacctgctaccgcgcctttgcttcctggcgcacgcggagcctcctggagcctgccaccatcctgcctactacgtgctgccctgcgcccgcagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgccgcaccctggccgccttccccaccacctgcctggagaggtaaggggaggttgtccaactgcttgggggaagtcccaccaactcttagggagtggagcggcaggcctggggacctgggtgggcgcggggatggggcgctagaggcttgagtagagaggtagaagcgcacttgatgccacgtgctcagattccaggagcagggagaaagcaaggggaagggaagcaagggcccgcggcttgctgccatccccatcctctgacaaactgcagtcgtgctcaggtgacctgctgaagacccggctgcgggaaggccacgttgacatttccaggcagcactgagcctgcccatttcttaagcatttgtttttcattcctcagaccagagctaggggcattgcctcctctgcccctggaagacagggagatttttttttttttggtggggtgtgtgggggtgggggctcccactgttgagggtgtgtccatattccagaggcggccgc

SEQ ID NO: 17 - DNA, 1799 bp, synthetic construct
  source 1..1799: mol_type = other DNA
gcggccgccgatcaaagaaagccctgtgtgcctcccagacccaccacccccgtgcaaactctctcaacacgacaccgctgcgtgactttcctgctgttactagaggtcggcaggtacttttccctaggaaactgggtgtgcacgtctcatccccgtgccaattctcttgccgcttttccttccacacgcggcgtcctctgggcggagggtgcagctccgcgtgctccagctgccgcgaaagttgcacccggcccagtctggctgcgggccccgcctccgccttctcagcgcccagccccaccctccgattggtcagcagtgcccagccccgccccgctcctccgaggggatttaaagcagcggccggtcagtctgggcgcttgcaggctgctaaacccaaccgcagttgactagcacctgctaccgcgcctttgcttcctggcgcacgcggagcctcctggagcctgccaccatcctgcctactacgtgctgccctgcgcccgcagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccggagctactaacttcagcctgctgaagcaggctggcgacgtggaggagaaccctggacctatgtgccgcaccctggccgccttccccaccacctgcctggagaggtaaggggaggttgtccaactgcttgggggaagtcccaccaactcttagggagtggagcggcaggcctggggacctgggtgggcgcggggatggggcgctagaggcttgagtagagaggtagaagcgcacttgatgccacgtgctcagattccaggagcagggagaaagcaaggggaagggaagcaagggcccgcggcttgctgccatccccatcctctgacaaactgcagtcgtgctcaggtgacctgctgaagacccggctgcgggaaggccacgttgacatttccaggcagcactgagcctgcccatttcttaagcatttgtttttcattcctcagaccagagctaggggcattgcctcctctgcccctggaagacagggagatttttttttttttggtggggtgtgtgggggtgggggctcccactgttgagggtgtgtccatattccagaggcggccgc

SEQ ID NO: 18 - DNA, 1802 bp, synthetic construct
  source 1..1802: mol_type = other DNA
gcggccgcgtgacttgaagcagggttgctgtagaagaaagggtgaaaggggattgcgtttgtatacattcttgatagatcctcatgttaattctgagagattggaggacagacactggatcagatgtgggtacaagcccagagctgaaaagctagtgatcaactcaggagcccccacttcctgatgcagcatccgatgctctttccaatgcccagcctgccgttggccctccctcagccatgcccaccctcttccccaactcaccctgtgtgtgtcctccttcccgcaggtcaacattgaccatgagacccacgagctgacgaggatgaacctgcagactgccacagccacatgctttgatgcggctcaggggaagacacgtaccctgatggagaaggactcctacccacgcttcctgaagtcgcctgcttaccgggacctggctgcccaagcctcagccgcctctgccactctgtccagctgcagcctggacgagccctcacacaccggatccggagctactaacttcagcctgctgaagcaggctggcgacgtggaggagaaccctggacctatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatgagtctccacggcagtgagtaagccagccgggaagagaggttgagtcacccatccccgaggtggctgcccctgtgtgggaggcaggttctgcaaagcaagtgcaagaggacaaaaaaaaaaaaaaaaaaaaaaaaaatgcgctccagcagcctgtttgggaagcagcagtctctccttcagatactgtgggactcatgctggagaggagccgcccacttccaggacctgtgaataagggctaatgatgagggttggtggggctctctgtggggcaaaaaggtggtatgggggttagcactggctctcgttctcaccggagaaggaagtgttctagtgtggtttaggaaacatgtggataaagggaaccatgaaaatgagaggaggaaagacatccagatcagctgttttgcctgttgctcagttgactctgattgcatcctgttttcctaattcccagactgttctgggcacggaagggaccctggatgtggagtcttcgcggccgc

SEQ ID NO: 19 - DNA, 2075 bp, synthetic construct
  source 1..2075: mol_type = other DNA
gcggccgcctgcagaaaagaagagaactcttaaaatattttgatcagatgaattttatagtcaaggactatccaaagcatggagtaagatacaactctatgtgatgaatttctatgtgagtttctatatgataaattaatacgccaaatgaaacagtttattgaaacatgcaacttttttaaaataaacaaacatcctccagaaccaaagactgatgctgttaatgctttagacatgtgacatatgtgtgtgtacctctgtatgtttaattttctttctttctaatcaagtgacctgttctcgtttggttaagtttcaaccacaagacaccactgtatgctttttttttttcaagatacacgtcacagcacaccaagaaaaggggaacttccagtgtctgtggtaacatcacttgataaacagactcctttaaacagcaagtgcctgtctgcattctactatataaagcagcagagacgttgactagcgcatatttgctaagagcaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggatcctgaccatgtacgattgcgcgaacataagaaggctaatgcgtactgactaggcgcgcagcagccatctttactccaaagttagacaaaatgccaggaatgttcttctctgctaacccaaaggaattgaaaggaaccactcattcacttctagacgacaaaatgcaaaaaaggaggccaaagactttgtaagtttgttcagagtctcactttgtgaattttaacaagttatctttaatggcagaaggattgagaatgaatggaagagagtgtgagcatttagtttagccagatcagaggagttaattgcctgttgtgagtaataagtaatattcagctgcattaaaaaataaatcttgatatggcattaaattgggtaggagtttttctctacacaggctcataaaaatgggttctatagcaaaatgttgtatcaaccatttttagtattgccttattccttagagaaatttattctatgtctttttacaaataaaatgaattctgaagaaatatgaatattcacttttatgtcttttgtagtggaatgggcggccgc

SEQ ID NO: 20 - DNA, 2075 bp, synthetic construct
  source 1..2075: mol_type = other DNA
gcggccgcactccggcctcgtggcgggcgacctcctcctgccagggaatcgccgctctggcctcgggctggggagcccgtcagggtgggtgagggagcgccggcgcccccgccgggccgccgccaacttcggctccctccctccgtcgcaaagccctcgagcccgcccggccgccacgcttcagcaaaaggtcgtgcgcagcggcccacactgaagactctccatctgctcccacacctgccccagctggccgctgctatgtggcccgagtgcgcaagaagccggggccgcagacgtcagcagcgccccggcttcgagacccttcggcagcagccgtgactgccgccggcgggcgctgacccatccccgtgccagtctgcagccgaccaatccgcgtcctcttgaggcggggccggagccgcgaggccccgcccccaagccgaggcctcataaatgctgcgacgcacgcccagccgcaaacagccgtgtctccagcgggagaacgataatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggatcctgaccatgtacgattgctacgacgtagaagagctaatgcgtactgactaggcaaagtgctatgttcttggctgttcaacacgactgcagacccatggacaagagcgcaggcagtggccacaagagcgaggagaagcgagaaaagatgaaacggaccctgtgagtatggctttcttccctctcccgccaccccctgccccacactgcaagctgcaaacgcggtactttcgggctcgcctttgacgttaggaaactagcctgagcctatgcagggaaaaaaaatcgaaaaggtcaatttgttaagtaaggttaaatctgggtgatgctcgggtacagtttaagaaccgagggagacagttgatatgagggcggtggttgatgcgctaagaaattgcgggttggctttttgtcctcctgcattcaaaatgacatcagaatcctgcggctgaagcgcgtccccagcattcatacgttgcatgatgagttctcatcagcttacacagctactggaaggtgatgctcttgctggttctgaatatactcgtttaagcggccgc

SEQ ID NO: 21 - DNA, 2075 bp, synthetic construct
  source 1..2075: mol_type = other DNA
gcggccgccacattgcagaggacgtggctggtctctgggatgcagtccctctgtggaggtggcatggggagggacggatgcatgacctaaggggggtattttcagtgtctgacatgatcgataccactctggacaaggaggccaggatgcagaaagcctgtgtgcctcgctgattgtcggggaggatgtggcttggacaagagcctggttcctccgatgccagggttcttgtttcttccactcaacattgctgtcctgcagtccctccctccctgcacctcctgccttcgctttcattcgaggtgtccatggcaagtctggtcatttccccccatttcctcaggaataaaagtgcagcagtgcctgctgtggggacagctgagggcagtgaggccctggggagctgctgcaggcagcaggtgggcgggacgccagcaggctgtctagctgttcccatgatggtctcctgttctctgcagaggcgtgcaaagactccagaattggaggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggatcctgaccatgtacgattggaaagtcaaagcactgctaatgcgtactgactaggatgaagactctgctgctgtttgtgtgtctgctgctgacctgggagagtgggcaggtcctgggggaccagacggtctcagacaatgagctccagggtgagtagaccaagcatgatgttcctctggccacagggtgatgaggtcagagggcagggtagctaattctgctcagtgcctctctatcaggccccagtgttacagaccgtttttatcttgtgcactgggtctgggtgcctgtgtctgggcccactctgagcctcagctccgaggcccctggttcaggctctgcgtgcatcagactgccggcatttgcaggcatttcccaagcactttcggctgttgcatttcattcagctcttcccctcccaggccccttagcccagctcccaggcctcctccacgaagctgtgtctggaccaccggagctcttatccctctcccctttggagtgcccagagcttatccctcctgtgagctgacggtttctgcaggatcattggcggccgc

SEQ ID NO: 22 - DNA, 2075 bp, synthetic construct
  source 1..2075: mol_type = other DNA
gcggccgctcactgcagcccaggaacctgagcccagagggggccacccaccttccccaggcagggaggcccggcccccagggagatgggggggatgggggaggagaagggcctgcccccacccggcagcctcaggaggggcagctcgggcgggatatggaaagaggccacagcagtgagcagagacacagaggaggaaggggccctgagctggggagacccccacggggtagggcgtgggggccacgggcccacctcctccccatctcctctgtctccctgtctctgtctctctctccctcccccaccctctccccagtcctaccccctcctcacccctcctcccccagcactgcctctgtcactctcgcccacgtggatgtggaggaagagggggcgggagcaaggggcgggcaccctcccttcaacctgacctgggacagtttcccttccgctcacctccgcctgagcagtggagaaggcggcactctggtggggctgctccaggcatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggatcctgaccatgtacgattgatcacatcgttgccagctaatgcgtactgactaggcagatcccacaagcgccctggccagtcgtctgggcggtgctacaactgggctggcggccaggatggttcttaggtaggtggggtcggcggtcaggtgtcccagagccaggggtctggagggaccttccaccctcagtccctggcaggtcggggggtgctgaggcgggcctggccctggcagcccaggggtcccggagcgaggggtctggagggacctttcactctcagtccctggcaggtcggggggtgctgtggcaggcccagccttggcccccagctctgccccttaccctgagctgtgtggctttgggcagctcgaactcctgggttcctctctgggccccaactcctcccctggcccaagtcccctctttgctcctgggcaggcaggacctctgtcccctctcagccggtccttggggctgcgtgtttctgtagaatgacgggtcaggctggccagaaccccaaaccttggccgtggggagtctgcgtggcgggcggccgc

SEQ ID NO: 23 - DNA, 2075 bp, synthetic construct
  source 1..2075: mol_type = other DNA
gcggccgcgcctgtaactcggctgaaggttagtgcaacttcatttctttcctttactctccagagctccccaaacatcaagaaacaggacaaggcaaaccctgtaacttaaggtttgcccgacccatcgccttcgggaacaactttctcattgtgaaattcaacttcatttctagatggtcatttctagaaagagactgctgaatctgagcttcagagaagaggctcatctgagtgggatgagtgggggggtatgagggagatgtttggaaatacccaggagtgtagaccctcagtagctttttagctctgggtctttatttggttagtctttccacgccctaaactgttgttctgcagcattctctctctcctgcctttcctctcgcgcccctacatgctctctgactgccgcgggctgccggtgtagctccaggtgtacccgagcccgggagaaagtgttcagttgaccaggctgagtgtgttatcaccctgtttcatttccagccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggatcctgaccatgtacgattgattcgccttacgcaagctaatgcgtactgactaggccttgtgttcaggcgcagtatgggtcctcgcctcaaggagccagccccgcttctcagagctacagttaccactcttcgggagaatacagctccgatttcttaactccagagtttgtcaagtttagcatggacctcaccaacactgaaatcactgccaccacttctctccccagcttcagtacctttatggacaactacagcacaggctacgacgtcaagccaccttgcttgtaccaaatgcccctgtccggacagcagtcctccattaaggtagaagacattcagatgcacaactaccagcaacacagccacctgcccccccagtctgaggagatgatgccgcactccgggtcggtttactacaagccctcctcgcccccgacgcccaccaccccgggcttccaggtgcagcacagccccatgtgggacgacccgggatctctccacaacttccaccagaactacgtggccactacgcacatgatcgagcagaggaagcggccgc

SEQ ID NO: 24 - DNA, 2075 bp, synthetic construct
  source 1..2075: mol_type = other DNA
gcggccgcgtttgcccaagagcaggagtgcttggtggtggtctgaggggaggagctacagacttgtcagggctactttgagcccttaactggcatccagtactccccaaatccagtgctgttcagggtggcctcagaatgaaacgggagcttaagttggttttgaggagcagtacctgttgctgacagacagggttggctccctgacaaacattactgaaaacacgggttattcccctcctcctcagtacccactctctgccttccttctcttgtttaggctaatcttttaaagtgatgtaacttaaaacagtccagtgtgagttaatctctggggccagctagtatcccgggagtagaggtgctaagatcttttgcctacagatcagggtaatgacaagatcaaacactggggtttcctgcaggcagctatagaggagtcgtattaaagtcaggctaatagaatggctttttttttttcctttccttttcaggtgttggagagcacaatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggatcctgaccatgtacgattgctttgtacgagcaaagctaatgcgtactgactagggctgaacaagtccttcctctgtctttgtatttgagcaatatgcggaaagctgtgaagatacgggagagaactccagaagacatttttaaacctactaatgggatcattcatcattttaaaaccatgcaccgatacacactggaaatgttcagaacttgccagttttgtcctcagtttcgggagatcatccacaaagccctcatcgacagaaacatccaggccaccctggaaagccagaagaaactcaactggtgtcgagaagtccggaagcttgtggcgctgaaaacgaacggtaagacttgttctgttgtgtttcttttgcctgggtgatagctcccgcctgctggatccccattcatgaagctttaataggacaagcccaaactcaaatcaatcttgagatttagtattgagacctttatatagaatctctattcggggtatgtgataatagcagacttgttttgtgaatctatttattaaggatattttctgatgcggccgc

SEQ ID NO: 25 - DNA, 864 bp, Homo sapiens
  source 1..864: mol_type = other DNA
atggacgggccgcgcctgctgctgttgctgcttctgggggtgtcccttggaggtgccaaggaggcatgccccacaggcctgtacacacacagcggtgagtgctgcaaagcctgcaacctgggcgagggtgtggcccagccttgtggagccaaccagaccgtgtgtgagccctgcctggacagcgtgacgttctccgacgtggtgagcgcgaccgagccgtgcaagccgtgcaccgagtgcgtggggctccagagcatgtcggcgccgtgcgtggaggccgacgacgccgtgtgccgctgcgcctacggctactaccaggatgagacgactgggcgctgcgaggcgtgccgcgtgtgcgaggcgggctcgggcctcgtgttctcctgccaggacaagcagaacaccgtgtgcgaggagtgccccgacggcacgtattccgacgaggccaaccacgtggacccgtgcctgccctgcaccgtgtgcgaggacaccgagcgccagctccgcgagtgcacacgctgggccgacgccgagtgcgaggagatccctggccgttggattacacggtccacacccccagagggctcggacagcacagcccccagcacccaggagcctgaggcacctccagaacaagacctcatagccagcacggtggcaggtgtggtgaccacagtgatgggcagctcccagcccgtggtgacccgaggcaccaccgacaacctcatccctgtctattgctccatcctggctgctgtggttgtgggccttgtggcctacatagccttcaagaggtggaacagtcatcgctatcctcgaggtcaccgcggtctagagtccacctgcagccaagcttatcgataa

SEQ ID NO: 26 - DNA, 378 bp, Homo sapiens
  source 1..378: mol_type = other DNA
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacataa

SEQ ID NO: 27 - DNA, 297 bp, Homo sapiens
  source 1..297: mol_type = other DNA
atgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaa

SEQ ID NO: 28 - DNA, 738 bp, synthetic construct
  source 1..738: mol_type = other DNA
atgaagaaaagtggtgttcttttcctcttgggcatcatcttgctggttctgattggagtgcaaggaaccccagtagtgagaaagggtcgctgttcctgcatcagcaccaaccaagggactatccacctacaatccttgaaagaccttaaacaatttgccccaagcccttcctgcgagaaaattgaaatcattgctacactgaagaatggagttcaaacatgtctaaacccagattcagcagatgtgaaggaactgattaaaaagtgggagaaacaggtcagccaaaagaaaaagcaaaagaatgggaaaaaacatcaaaaaaagaaagttctgaaagttcgaaaatctcaacgttctcgtcaaaagaagactacaggatccggagctactaacttcagcctgctgaagcaggctggcgacgtggaggagaaccctggacctatgaatcaaactgccattctgatttgctgccttatctttctgactctaagtggcattcaaggagtacctctctctagaactgtacgctgtacctgcatcagcattagtaatcaacctgttaatccaaggtctttagaaaaacttgaaattattcctgcaagccaattttgtccacgtgttgagatcattgctacaatgaaaaagaagggtgagaagagatgtctgaatccagaatcgaaggccatcaagaatttactgaaagcagttagcaaggaaaggtctaaaagatctccttaa

SEQ ID NO: 29 - DNA, 702 bp, Homo sapiens
  source 1..702: mol_type = other DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtga

SEQ ID NO: 30 - DNA, 501 bp, Homo sapiens
  source 1..501: mol_type = other DNA
atgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 31 - DNA, 1266 bp, synthetic construct
  source 1..1266: mol_type = other DNA
atgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgggatccggagctactaacttcagcctgctgaagcaggctggcgacgtggaggagaaccctggacctatgaaatatacaagttatatcttggcttttcagctctgcatcgttttgggttctcttggctgttactgccaggacccatatgtaaaagaagcagaaaaccttaagaaatattttaatgcaggtcattcagatgtagcggataatggaactcttttcttaggcattttgaagaattggaaagaggagagtgacagaaaaataatgcagagccaaattgtctccttttacttcaaactttttaaaaactttaaagatgaccagagcatccaaaagagtgtggagaccatcaaggaagacatgaatgtcaagtttttcaatagcaacaaaaagaaacgagatgacttcgaaaagctgactaattattcggtaactgacttgaatgtccaacgcaaagcaatacatgaactcatccaagtgatggctgaactgtcgccagcagctaaaacagggaagcgaaaaaggagtcagatgctgtttcgaggtcgaagagcatcccagtaa

SEQ ID NO: 32 - DNA, 462 bp, Homo sapiens
  source 1..462: mol_type = other DNA
atgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttga

SEQ ID NO: 33 - DNA, 1812 bp, Homo sapiens
  source 1..1812: mol_type = other DNA
atgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaa

SEQ ID NO: 34 - DNA, 1599 bp, Homo sapiens
  source 1..1599: mol_type = other DNA
atgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggtggcggtggcggcggatctagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttcctaa

SEQ ID NO: 35 - DNA, 63 bp, synthetic construct
  source 1..63: mol_type = other DNA
tccggagctactaacttcagcctgctgaagcaggctggcgacgtggaggagaaccctggacct

SEQ ID NO: 36 - DNA, 63 bp, synthetic construct
  source 1..63: mol_type = other DNA
ggctccggcgagggcaggggaagtcttctaacatgcggggacgtggaggaaaatcccggccca

SEQ ID NO: 37 - DNA, 284 bp, synthetic construct
  source 1..284: mol_type = other DNA
tctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 38 - RNA, 20 nt, synthetic construct
  source 1..20: mol_type = other RNA
ggacgcaggacagcacgtcg

SEQ ID NO: 39 - DNA, 1759 bp, synthetic construct
  source 1..1759: mol_type = other DNA
gcggccgccgatcaaagaaagccctgtgtgcctcccagacccaccacccccgtgcaaactctctcaacacgacaccgctgcgtgactttcctgctgttactagaggtcggcaggtacttttccctaggaaactgggtgtgcacgtctcatccccgtgccaattctcttgccgcttttccttccacacgcggcgtcctctgggcggagggtgcagctccgcgtgctccagctgccgcgaaagttgcacccggcccagtctggctgcgggccccgcctccgccttctcagcgcccagccccaccctccgattggtcagcagtgcccagccccgccccgctcctccgaggggatttaaagcagcggccggtcagtctgggcgcttgcaggctgctaaacccaaccgcagttgactagcacctgctaccgcgcctttgcttcctggcgcacgcggagcctcctggagcctgccaccatcctgcctactacgtgctgccctgcgcccgcagccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgccgcaccctggccgccttccccaccacctgcctggagaggtaaggggaggttgtccaactgcttgggggaagtcccaccaactcttagggagtggagcggcaggcctggggacctgggtgggcgcggggatggggcgctagaggcttgagtagagaggtagaagcgcacttgatgccacgtgctcagattccaggagcagggagaaagcaaggggaagggaagcaagggcccgcggcttgctgccatccccatcctctgacaaactgcagtcgtgctcaggtgacctgctgaagacccggctgcgggaaggccacgttgacatttccaggcagcactgagcctgcccatttcttaagcatttgtttttcattcctcagaccagagctaggggcattgcctcctctgcccctggaagacagggagatttttttttttttggtggggtgtgtgggggtgggggctcccactgttgagggtgtgtccatattccagaggcggccgc

SEQ ID NO: 40 - DNA, 3109 bp, synthetic construct
  source 1..3109: mol_type = other DNA
gcggccgccgatcaaagaaagccctgtgtgcctcccagacccaccacccccgtgcaaactctctcaacacgacaccgctgcgtgactttcctgctgttactagaggtcggcaggtacttttccctaggaaactgggtgtgcacgtctcatccccgtgccaattctcttgccgcttttccttccacacgcggcgtcctctgggcggagggtgcagctccgcgtgctccagctgccgcgaaagttgcacccggcccagtctggctgcgggccccgcctccgccttctcagcgcccagccccaccctccgattggtcagcagtgcccagccccgccccgctcctccgaggggatttaaagcagcggccggtcagtctgggcgcttgcaggctgctaaacccaaccgcagttgactagcacctgctaccgcgcctttgcttcctggcgcacgcggagcctcctggagcctgccaccatcctgcctactacgtgctgccctgcgcccgcagccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagttaatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgccgcaccctggccgccttccccaccacctgcctggagaggtaaggggaggttgtccaactgcttgggggaagtcccaccaactcttagggagtggagcggcaggcctggggacctgggtgggcgcggggatggggcgctagaggcttgagtagagaggtagaagcgcacttgatgccacgtgctcagattccaggagcagggagaaagcaaggggaagggaagcaagggcccgcggcttgctgccatccccatcctctgacaaactgcagtcgtgctcaggtgacctgctgaagacccggctgcgggaaggccacgttgacatttccaggcagcactgagcctgcccatttcttaagcatttgtttttcattcctcagaccagagctaggggcattgcctcctctgcccctggaagacagggagatttttttttttttggtggggtgtgtgggggtgggggctcccactgttgagggtgtgtccatattccagaggcggccgc

SEQ ID NO: 41 - DNA, 1541 bp, synthetic construct
  source 1..1541: mol_type = other DNA
gcggccgccgatcaaagaaagccctgtgtgcctcccagacccaccacccccgtgcaaactctctcaacacgacaccgctgcgtgactttcctgctgttactagaggtcggcaggtacttttccctaggaaactgggtgtgcacgtctcatccccgtgccaattctcttgccgcttttccttccacacgcggcgtcctctgggcggagggtgcagctccgcgtgctccagctgccgcgaaagttgcacccggcccagtctggctgcgggccccgcctccgccttctcagcgcccagccccaccctccgattggtcagcagtgcccagccccgccccgctcctccgaggggatttaaagcagcggccggtcagtctgggcgcttgcaggctgctaaacccaaccgcagttgactagcacctgctaccgcgcctttgcttcctggcgcacgcggagcctcctggagcctgccaccatcctgcctactacgtgctgccctgcgcccgcagccatgtacaggatgcaactcctgtcttgcattgcactaagtcttgcacttgtcacaaacagtgcacctacttcaagttctacaaagaaaacacagctacaactggagcatttactgctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgactggatccggagctactaacttcagcctgctgaagcaggctggcgacgtggaggagaaccctggacctatgtgccgcaccctggccgccttccccaccacctgcctggagaggtaaggggaggttgtccaactgcttgggggaagtcccaccaactcttagggagtggagcggcaggcctggggacctgggtgggcgcggggatggggcgctagaggcttgagtagagaggtagaagcgcacttgatgccacgtgctcagattccaggagcagggagaaagcaaggggaagggaagcaagggcccgcggcttgctgccatccccatcctctgacaaactgcagtcgtgctcaggtgacctgctgaagacccggctgcgggaaggccacgttgacatttccaggcagcactgagcctgcccatttcttaagcatttgtttttcattcctcagaccagagctaggggcattgcctcctctgcccctggaagacagggagatttttttttttttggtggggtgtgtgggggtgggggctcccactgttgagggtgtgtccatattccagaggcggccgc

SEQ ID NO: 42 - DNA, 2891 bp, synthetic construct
  source 1..2891: mol_type = other DNA
gcggccgccgatcaaagaaagccctgtgtgcctcccagacccaccacccccgtgcaaactctctcaacacgacaccgctgcgtgactttcctgctgttactagaggtcggcaggtacttttccctaggaaactgggtgtgcacgtctcatccccgtgccaattctcttgccgcttttccttccacacgcggcgtcctctgggcggagggtgcagctccgcgtgctccagctgccgcgaaagttgcacccggcccagtctggctgcgggccccgcctccgccttctcagcgcccagccccaccctccgattggtcagcagtgcccagccccgccccgctcctccgaggggatttaaagcagcggccggtcagtctgggcgcttgcaggctgctaaacccaaccgcagttgactagcacctgctaccgcgcctttgcttcctggcgcacgcggagcctcctggagcctgccaccatcctgcctactacgtgctgccctgcgcccgcagccatgtggccccctgggtcagcctcccagccaccgccctcacctgccgcggccacaggtctgcatccagcggctcgccctgtgtccctgcagtgccggctcagcatgtgtccagcgcgcagcctcctccttgtggctaccctggtcctcctggaccacctcagtttggccagaaacctccccgtggccactccagacccaggaatgttcccatgccttcaccactcccaaaacctgctgagggccgtcagcaacatgctccagaaggccagacaaactctagaattttacccttgcacttctgaagagattgatcatgaagatatcacaaaagataaaaccagcacagtggaggcctgtttaccattggaattaaccaagaatgagagttgcctaaattccagagagacctctttcataactaatgggagttgcctggcctccagaaagacctcttttatgatggccctgtgccttagtagtatttatgaagacttgaagatgtaccaggtggagttcaagaccatgaatgcaaagcttctgatggatcctaagaggcagatctttctagatcaaaacatgctggcagttattgatgagctgatgcaggccctgaatttcaacagtgagactgtgccacaaaaatcctcccttgaagaaccggatttttataaaactaaaatcaagctctgcatacttcttcatgctttcagaattcgggcagtgactattgatagagtgatgagctatctgaatgcttccggaagcggagctactaacttcagcctgctgaagcaggctggagacgtggaggagaaccctggacctatgtgtcaccagcagttggtcatctcttggttttccctggtttttctggcatctcccctcgtggccatatgggaactgaagaaagatgtttatgtcgtagaattggattggtatccggatgcccctggagaaatggtggtcctcacctgtgacacccctgaagaagatggtatcacctggaccttggaccagagcagtgaggtcttaggctctggcaaaaccctgaccatccaagtcaaagagtttggagatgctggccagtacacctgtcacaaaggaggcgaggttctaagccattcgctcctgctgcttcacaaaaaggaagatggaatttggtccactgatattttaaaggaccagaaagaacccaaaaataagacctttctaagatgcgaggccaagaattattctggacgtttcacctgctggtggctgacgacaatcagtactgatttgacattcagtgtcaaaagcagcagaggctcttctgacccccaaggggtgacgtgcggagctgctacactctctgcagagagagtcagaggggacaacaaggagtatgagtactcagtggagtgccaggaggacagtgcctgcccagctgctgaggagagtctgcccattgaggtcatggtggatgccgttcacaagctcaagtatgaaaactacaccagcagcttcttcatcagggacatcatcaaacctgacccacccaagaacttgcagctgaagccattaaagaattctcggcaggtggaggtcagctgggagtaccctgacacctggagtactccacattcctacttctccctgacattctgcgttcaggtccagggcaagagcaagagagaaaagaaagatagagtcttcacggacaagacctcagccacggtcatctgccgcaaaaatgccagcattagcgtgcgggcccaggaccgctactatagctcatcttggagcgaatgggcatctgtgccctgcagtggatccggagctactaacttcagcctgctgaagcaggctggcgacgtggaggagaaccctggacctatgtgccgcaccctggccgccttccccaccacctgcctggagaggtaaggggaggttgtccaactgcttgggggaagtcccaccaactcttagggagtggagcggcaggcctggggacctgggtgggcgcggggatggggcgctagaggcttgagtagagaggtagaagcgcacttgatgccacgtgctcagattccaggagcagggagaaagcaaggggaagggaagcaagggcccgcggcttgctgccatccccatcctctgacaaactgcagtcgtgctcaggtgacctgctgaagacccggctgcgggaaggccacgttgacatttccaggcagcactgagcctgcccatttcttaagcatttgtttttcattcctcagaccagagctaggggcattgcctcctctgcccctggaagacagggagatttttttttttttggtggggtgtgtgggggtgggggctcccactgttgagggtgtgtccatattccagaggcggccgc

SEQ ID NO: 43 - DNA, 1492 bp, synthetic construct
  source 1..1492: mol_type = other DNA
gcggccgcctggtcttcaagccttcctcttctcctggcaccccaagtctccgaccttgggggtggggacagccccgtgcgcttgagatgaggccctgggggctaccagtcgtgccccaccccttcagactcccctcgctgcctcctccctcccaacgcccagccccactctcttattggtcggcgacgttcgtccccgccccgccccacggggataaaagccttagtagccaccggcccggcgcgtgccgctctccagacctcttctgcgcagcctggtacttgctactcgcttttccctcttcgccctccggtccgccaccatcctctttacgacgtgctgtcctgcgtccgcaaccatgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgccgcaccctagccaccttccccaacacctgcctggagaggtaagaggattttttttttttttttagcagtccggacgaccaagtcccgaatccactaaccctcagccttgggaacgcggggaaacggggacagccgggcagaacttgttgtgctataaagaccccaaagtgttaagacctatcagggcatagcccaagtgctgcctttgtggcctagaacttgaagactccaatgagagaagcgcttagggccttgggaatggtcaagtgtcccaggtttcttgggactgaactctgggtggtggtgttagttgggaggccctccttccccaacttctcggtgctggcgcggccgc

SEQ ID NO: 44 - DNA, 3414 bp, synthetic construct
  source 1..3414: mol_type = other DNA
gcggccgcggttgcagagatttctgctataattgtgttaagtaaatgtttccgcccatgcctgctgatcttggccaaatcgctagttttctgatcttctgatccttcatctcaaagtggaggtaatgaccgtgggggcgaatagctcacttggctcccgtcaccttcgaagaccatgggatggtacctgttctgacctgagagagagtgcagtatagtggtgatgtactttttcttggaatgcaccccatgcgcctgtcctccgtgaactctccaccccctaccaaacctctcctgtcacttttgaattgttttcctgcttttactagaggtcggcgtctctctctctctctctctctctctctctctctctctctctctctctctctctctctctctcggcgtatgtctggtcttcaagccttcctcttctcctggcaccccaagtctccgaccttgggggtggggacagccccgtgcgcttgagatgaggccctgggggctaccagtcgtgccccaccccttcagactcccctcgctgcctcctccctcccaacgcccagccccactctcttattggtcggcgacgttcgtccccgccccgccccacggggataaaagccttagtagccaccggcccggcgcgtgccgctctccagacctcttctgcgcagcctggtacttgctactcgcttttccctcttcgccctccggtccgccaccatcctctttacgacgtgctgtcctgcgtccgcaaccatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatctagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctgatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgccgcaccctagccaccttccccaacacctgcctggagaggtaagaggattttttttttttttttagcagtccggacgaccaagtcccgaatccactaaccctcagccttgggaacgcggggaaacggggacagccgggcagaacttgttgtgctataaagaccccaaagtgttaagacctatcagggcatagcccaagtgctgcctttgtggcctagaacttgaagactccaatgagagaagcgcttagggccttgggaatggtcaagtgtcccaggtttcttgggactgaactctgggtggtggtgttagttgggaggccctccttccccaacttctcggtgctggcaaacaaacagaatccttacaccttgatggtttttctttcagagccaaagagttcaagacgcggctgggaatctttcttcataaatcagagctgagctccgatactgggggtattagcaaattcgagtgggccagtaagcataacaaagagaggtaagtcctattgtctgtccagccctggggctttagaggggagaaggcctgaaagggaaaataggagcagactagctaggtgcccgaaacctctctctggagatttgagtgagactttactgctgtcctgtggctgcggagccccattgtggaggtggtagagtccaggtttgccacccccagcagatgcccgcgtgcaggacatgcatccctgtttctctgaggatgttgtaacactcgaatacagggcggccgc

SEQ ID NO: 45 - DNA, 1289 bp, synthetic construct
  source 1..1289: mol_type = other DNA
gcggccgcctggtcttcaagccttcctcttctcctggcaccccaagtctccgaccttgggggtggggacagccccgtgcgcttgagatgaggccctgggggctaccagtcgtgccccaccccttcagactcccctcgctgcctcctccctcccaacgcccagccccactctcttattggtcggcgacgttcgtccccgccccgccccacggggataaaagccttagtagccaccggcccggcgcgtgccgctctccagacctcttctgcgcagcctggtacttgctactcgcttttccctcttcgccctccggtccgccaccatcctctttacgacgtgctgtcctgcgtccgcaaccatgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaaggatccggagctactaacttcagcctgctgaagcaggctggcgacgtggaggagaaccctggacctatgtgccgcaccctagccaccttccccaacacctgcctggagaggtaagaggattttttttttttttttagcagtccggacgaccaagtcccgaatccactaaccctcagccttgggaacgcggggaaacggggacagccgggcagaacttgttgtgctataaagaccccaaagtgttaagacctatcagggcatagcccaagtgctgcctttgtggcctagaacttgaagactccaatgagagaagcgcttagggccttgggaatggtcaagtgtcccaggtttcttgggactgaactctgggtggtggtgttagttgggaggccctccttccccaacttctcggtgctgcggccgc

SEQ ID NO: 46 - DNA, 3211 bp, synthetic construct
  source 1..3211: mol_type = other DNA
gcggccgcggttgcagagatttctgctataattgtgttaagtaaatgtttccgcccatgcctgctgatcttggccaaatcgctagttttctgatcttctgatccttcatctcaaagtggaggtaatgaccgtgggggcgaatagctcacttggctcccgtcaccttcgaagaccatgggatggtacctgttctgacctgagagagagtgcagtatagtggtgatgtactttttcttggaatgcaccccatgcgcctgtcctccgtgaactctccaccccctaccaaacctctcctgtcacttttgaattgttttcctgcttttactagaggtcggcgtctctctctctctctctctctctctctctctctctctctctctctctctctctctctctctcggcgtatgtctggtcttcaagccttcctcttctcctggcaccccaagtctccgaccttgggggtggggacagccccgtgcgcttgagatgaggccctgggggctaccagtcgtgccccaccccttcagactcccctcgctgcctcctccctcccaacgcccagccccactctcttattggtcggcgacgttcgtccccgccccgccccacggggataaaagccttagtagccaccggcccggcgcgtgccgctctccagacctcttctgcgcagcctggtacttgctactcgcttttccctcttcgccctccggtccgccaccatcctctttacgacgtgctgtcctgcgtccgcaaccatgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatctagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgccggatccggagctactaacttcagcctgctgaagcaggctggcgacgtggaggagaaccctggacctatgtgccgcaccctagccaccttccccaacacctgcctggagaggtaagaggattttttttttttttttagcagtccggacgaccaagtcccgaatccactaaccctcagccttgggaacgcggggaaacggggacagccgggcagaacttgttgtgctataaagaccccaaagtgttaagacctatcagggcatagcccaagtgctgcctttgtggcctagaacttgaagactccaatgagagaagcgcttagggccttgggaatggtcaagtgtcccaggtttcttgggactgaactctgggtggtggtgttagttgggaggccctccttccccaacttctcggtgctggcaaacaaacagaatccttacaccttgatggtttttctttcagagccaaagagttcaagacgcggctgggaatctttcttcataaatcagagctgagctccgatactgggggtattagcaaattcgagtgggccagtaagcataacaaagagaggtaagtcctattgtctgtccagccctggggctttagaggggagaaggcctgaaagggaaaataggagcagactagctaggtgcccgaaacctctctctggagatttgagtgagactttactgctgtcctgtggctgcggagccccattgtggaggtggtagagtccaggtttgccacccccagcagatgcccgcgtgcaggacatgcatccctgtttctctgaggatgttgtaacactcgaatacgcggccgc

SEQ ID NO: 47 - DNA, 1702 bp, synthetic construct
  source 1..1702: mol_type = other DNA
gcggccgcctggtcttcaagccttcctcttctcctggcaccccaagtctccgaccttgggggtggggacagccccgtgcgcttgagatgaggccctgggggctaccagtcgtgccccaccccttcagactcccctcgctgcctcctccctcccaacgcccagccccactctcttattggtcggcgacgttcgtccccgccccgccccacggggataaaagccttagtagccaccggcccggcgcgtgccgctctccagacctcttctgcgcagcctggtacttgctactcgcttttccctcttcgccctccggtccgccaccatcctctttacgacgtgctgtcctgcgtccgcaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaatctagagggccctattgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtgccgcaccctagccaccttccccaacacctgcctggagaggtaagaggattttttttttttttttagcagtccggacgaccaagtcccgaatccactaaccctcagccttgggaacgcggggaaacggggacagccgggcagaacttgttgtgctataaagaccccaaagtgttaagacctatcagggcatagcccaagtgctgcctttgtggcctagaacttgaagactccaatgagagaagcgcttagggccttgggaatggtcaagtgtcccaggtttcttgggactgaactctgggtggtggtgttagttgggaggccctccttccccaacttctcggtgctggcgcggccgc

SEQ ID NO: 48 - DNA, 1499 bp, synthetic construct
  source 1..1499: mol_type = other DNA
gcggccgcctggtcttcaagccttcctcttctcctggcaccccaagtctccgaccttgggggtggggacagccccgtgcgcttgagatgaggccctgggggctaccagtcgtgccccaccccttcagactcccctcgctgcctcctccctcccaacgcccagccccactctcttattggtcggcgacgttcgtccccgccccgccccacggggataaaagccttagtagccaccggcccggcgcgtgccgctctccagacctcttctgcgcagcctggtacttgctactcgcttttccctcttcgccctccggtccgccaccatcctctttacgacgtgctgtcctgcgtccgcaaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagggatccggagctactaacttcagcctgctgaagcaggctggcgacgtggaggagaaccctggacctatgtgccgcaccctagccaccttccccaacacctgcctggagaggtaagaggattttttttttttttttagcagtccggacgaccaagtcccgaatccactaaccctcagccttgggaacgcggggaaacggggacagccgggcagaacttgttgtgctataaagaccccaaagtgttaagacctatcagggcatagcccaagtgctgcctttgtggcctagaacttgaagactccaatgagagaagcgcttagggccttgggaatggtcaagtgtcccaggtttcttgggactgaactctgggtggtggtgttagttgggaggccctccttccccaacttctcggtgctgcggccgc

SEQ ID NO: 49 - DNA, 510 bp, Mus musculus
  source 1..510: mol_type = genomic DNA
atgtacagcatgcagctcgcatcctgtgtcacattgacacttgtgctccttgtcaacagcgcacccacttcaagctccacttcaagctctacagcggaagcacagcagcagcagcagcagcagcagcagcagcagcagcacctggagcagctgttgatggacctacaggagctcctgagcaggatggagaattacaggaacctgaaactccccaggatgctcaccttcaaattttacttgcccaagcaggccacagaattgaaagatcttcagtgcctagaagatgaacttggacctctgcggcatgttctggatttgactcaaagcaaaagctttcaattggaagatgctgagaatttcatcagcaatatcagagtaactgttgtaaaactaaagggctctgacaacacatttgagtgccaattcgatgatgagtcagcaactgtggtggactttctgaggagatggatagccttctgtcaaagcatcatctcaacaagccctcaataa

SEQ ID NO: 50 - DNA, 1632 bp, Mus musculus
  source 1..1632: mol_type = genomic DNA
atgtgtcctcagaagctaaccatctcctggtttgccatcgttttgctggtgtctccactcatggccatgtgggagctggagaaagacgtttatgttgtagaggtggactggactcccgatgcccctggagaaacagtgaacctcacctgtgacacgcctgaagaagatgacatcacctggacctcagaccagagacatggagtcataggctctggaaagaccctgaccatcactgtcaaagagtttctagatgctggccagtacacctgccacaaaggaggcgagactctgagccactcacatctgctgctccacaagaaggaaaatggaatttggtccactgaaattttaaaaaatttcaaaaacaagactttcctgaagtgtgaagcaccaaattactccggacggttcacgtgctcatggctggtgcaaagaaacatggacttgaagttcaacatcaagagcagtagcagttcccctgactctcgggcagtgacatgtggaatggcgtctctgtctgcagagaaggtcacactggaccaaagggactatgagaagtattcagtgtcctgccaggaggatgtcacctgcccaactgccgaggagaccctgcccattgaactggcgttggaagcacggcagcagaataaatatgagaactacagcaccagcttcttcatcagggacatcatcaaaccagacccgcccaagaacttgcagatgaagcctttgaagaactcacaggtggaggtcagctgggagtaccctgactcctggagcactccccattcctacttctccctcaagttctttgttcgaatccagcgcaagaaagaaaagatgaaggagacagaggaggggtgtaaccagaaaggtgcgttcctcgtagagaagacatctaccgaagtccaatgcaaaggcgggaatgtctgcgtgcaagctcaggatcgctattacaattcctcatgcagcaagtgggcatgtgttccctgcagggtccgatccggtggcggtggctcgggcggtggtgggtcgggtggcggcggatctagggtcattccagtctctggacctgccaggtgtcttagccagtcccgaaacctgctgaagaccacagatgacatggtgaagacggccagagaaaaactgaaacattattcctgcactgctgaagacatcgatcatgaagacatcacacgggaccaaaccagcacattgaagacctgtttaccactggaactacacaagaacgagagttgcctggctactagagagacttcttccacaacaagagggagctgcctgcccccacagaagacgtctttgatgatgaccctgtgccttggtagcatctatgaggacttgaagatgtaccagacagagttccaggccatcaacgcagcacttcagaatcacaaccatcagcagatcattctagacaagggcatgctggtggccatcgatgagctgatgcagtctctgaatcataatggcgagactctgcgccagaaacctcctgtgggagaagcagacccttacagagtgaaaatgaagctctgcatcctgcttcacgccttcagcacccgcgtcgtgaccatcaacagggtgatgggctatctgagctccgcctga

SEQ ID NO: 51 - DNA, 18 bp, synthetic construct
  source 1..18: mol_type = other DNA
gggctgcgatactgggag

SEQ ID NO: 52 - DNA, 19 bp, synthetic construct
  source 1..19: mol_type = other DNA
ttcaggaaagcgtggaagg

SEQ ID NO: 53 - DNA, 1476 bp, synthetic construct
  source 1..1476: mol_type = other DNA
atggccttaccagtgaccgccttgctcctgccgctggccttgctgctccacgccgccaggccgcaggtacaactgcagcagtcaggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcgattacaaggacgacgatgacaagaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatatctacatctgggcgcccttggccgggacttgtggggtccttctcctgtcactggttatcaccctttactgcaaacggggcagaaagaaactcctgtatatattcaaacaaccatttatgagaccagtacaaactactcaagaggaagatggctgtagctgccgatttccagaagaagaagaaggaggatgtgaactgagagtgaagttcagcaggagcgcagacgcccccgcgtacaagcagggccagaaccagctctataacgagctcaatctaggacgaagagaggagtacgatgttttggacaagagacgtggccgggaccctgagatggggggaaagccgagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 54 - DNA, 1848 bp, synthetic construct
  source 1..1848: mol_type = other DNA
atggcctccaggctgctgtgctgggtgctgctgtgcctgctgggagcaggaccagtgaaggcaggcgtgacccagacacctaggtacctgatcaagacccgcggccagcaggtgacactgtcttgcagcccaatcagcggccaccgctccgtgtcttggtaccagcagaccccaggacagggcctgcagttcctgtttgagtatttctccgagacacagaggaacaagggcaatttccctggccggttttctggcagacagtttagcaactcccgctctgagatgaacgtgagcaccctggagctgggcgatagcgccctgtacctgtgcgccagctccctggccgcaggaggctatttcaacgagcagttctttggaccaggaaccaggctgacagtgctggaggacctgagaaatgtgaccccccctaaggtgtccctgtttgagccttctaaggccgagatcgccaacaagcagaaggccaccctggtgtgcctggcaaggggcttctttccagatcacgtggagctgagctggtgggtgaatggcaaggaggtgcactccggcgtgtgcaccgacccacaggcctacaaggagagcaactactcctattgtctgtctagccggctgagagtgtccgccacattctggcacaacccaaggaatcacttccgctgccaggtgcagtttcacggcctgagcgaggaggataagtggccagagggctccccaaagccagtgacccagaatatctctgccgaggcatggggaagggcagactgtggaatcaccagcgcctcctatcagcagggcgtgctgagcgccacaatcctgtacgagatcctgctgggcaaggccaccctgtatgccgtgctggtgtccacactggtggtcatggctatggtgaagagaaagaactctagggcaaagcggagcggaagcggagcaaccaatttcagcctgctgaagcaggcaggcgatgtggaggagaaccctggaccaatggacaagatcctgggcgcctcttttctggtgctgtggctgcagctgtgctgggtgtccggacagcagaaggagaagtctgatcagcagcaggtgaagcagtctccccagagcctgatcgtgcagaagggcggcatcagcatcatcaactgtgcctacgagaataccgccttcgattactttccctggtatcagcagttcccaggcaagggacccgccctgctgatcgcaatcaggcctgacgtgagcgagaagaaggagggccgcttcacaatcagctttaataagtccgccaagcagttctccctgcacatcatggacagccagcccggcgattccgccacctacttttgtgcagcagaggcaggaaaccacaggggctccacactgggccggctgtatttcggcagaggcacccagctgacagtgtggcctaacatccagaatcccgagcctgccgtgtaccagctgaaggacccaagatcccaggattctaccctgtgcctgttcacagactttgattctcagatcaatgtgcctaagacaatggagagcggcacctttatcacagacaagtgcgtgctggacatgaaggctatggactccaagtctaacggcgccatcgcctggtctaatcagaccagcttcacatgccaggatatctttaaggagacaaacgccacatatccttcctctgacgtgccatgtgatgccaccctgacagagaagagcttcgagacagacatgaacctgaattttcagaacctgctggtcatcgtgctgcggatcctgctgctgaaggtggccggcttcaatctgctgatgacactgagactgtggagctcc

SEQ ID NO: 55 - DNA, 1638 bp, synthetic construct
  source 1..1638: mol_type = other DNA
atggattttcaggtgcagattttcagcttcctgctaatcagtgcctcagtcataatgtctagacaggtacaactgcagcagtcaggacctgaactgaagaagcctggagagacagtcaagatctcctgcaaggcctctgggtatcctttcacaaactatggaatgaactgggtgaagcaggctccaggacagggtttaaagtggatgggctggattaacacctccactggagagtcaacatttgctgatgacttcaagggacggtttgacttctctttggaaacctctgccaacactgcctatttgcagatcaacaacctcaaaagtgaagacatggctacatatttctgtgcaagatgggaggtttaccacggctacgttccttactggggccaagggaccacggtcaccgtttcctctggcggtggcggttctggtggcggtggctccggcggtggcggttctgacatccagctgacccagtctcacaaattcctgtccacttcagtaggagacagggtcagcatcacctgcaaggccagtcaggatgtgtataatgctgttgcctggtatcaacagaaaccaggacaatctcctaaacttctgatttactcggcatcctcccggtacactggagtcccttctcgcttcactggcagtggctctgggccggatttcactttcaccatcagcagtgtgcaggctgaagacctggcagtttatttctgtcagcaacattttcgtactccattcacgttcggctcggggacaaaattggagatcgaacaaaaactcatctcagaagaggatctgaatggggtcaccgtctcttcaggcggcggcggcagcggagattacaaggacgacgatgacaagggaggcggaggcagcgcgtcagtcatcagcaactcggtgatgtacttcagttctgtcgtgccagtccttcagaaagtgaactctactactaccaagccagtgctgcgaactccctcacctgtgcaccctaccgggacatctcagccccagagaccagaagattgtcggccccgtggctcagtgaaggggaccggattggatcctaagctgttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcacccgaaagccttaccagccctacgcccctgccagagactttgcagcgtaccgccccgccgagagagcaaaattcagcaggagtgctgagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagtactgccaccaaggacacctatgatgccctgcatatgcagaccctggcccctcgctaa

(19) United States
(12) Patent Application Publication
(10) Pub. No.: US 2026/0286303 A1
(43) Pub. Date: Sep. 24, 2026

(54) COMPOSITIONS AND METHODS FOR IMMUNOTHERAPY - II

(71) Applicant: Peter MacCallum Cancer Institute, Melbourne (AU)

(72) Inventors: Paul Beavis, Reservoir (AU); Kah Min Yap, Carlton (AU); Xi Ying Amanda Chen, West Melbourne (AU); Phillip K. Darcy, Yallambie (AU)

(21) Appl. No.: 19/476,620

(22) PCT Filed: Apr. 19, 2024
(86) PCT No.: PCT/AU2024/050379

§ 371 Date: Oct. 17, 2025

(30) Foreign Application Priority Data
Apr. 20, 2023 (AU) 2023901180

(51) Int. Cl. C12N 5/0783; C07K 14/47

(52) U.S. Cl. CPC C12N 5/0636; C07K 14/4703; C12N 2510/00

(57) ABSTRACT

The present disclosure provides an engineered T cell comprising a heterologous nucleotide sequence encoding at least one immunomodulatory factor, wherein the heterologous nucleotide sequence is introduced in-frame of an endogenous gene of the T cell, wherein expression of the endogenous gene is under the control of endogenous regulatory elements, wherein expression of the heterologous nucleotide sequence is controlled by the endogenous regulatory elements of the endogenous gene, and wherein the endogenous gene is selected from the group consisting of RGS16, RGS1, RGS2, and CLU. In some embodiments, there is provided pharmaceutical compositions comprising the engineered T cell disclosed herein, and methods for the treatment of cancer using the engineered T cell disclosed herein. The present disclosure also provides genome editing systems, and methods for enhancing the function of a T cell using the genome editing systems disclosed herein.

### RELATED APPLICATIONS

This application claims priority from Australian Provisional Patent Application No. 2023901180 filed on 20 Apr. 2023, the entire content of which is incorporated by reference.

### FIELD

The present disclosure relates generally to T cells, e.g., CAR T cells, which have been engineered to express an immunomodulatory factor, including to express at least one immunomodulatory factor in a tumor selective manner. The present disclosure also extends to therapeutic uses of the T cells contemplated herein, and genome editing systems for engineering such T cells.

### BACKGROUND

Chimeric antigen receptor (CAR) T cell therapy has proven to be highly efficacious in the treatment of hematological malignancies, with a number of Food and Drug Administration (FDA) approved CAR T cell products currently available, including Kymriah (tisagenlecleucel), Yescarta (axicabtagene ciloleucel), Tecartus (brexucabtagene autolecucel), Abecma (idecabtagene vicleucel), Carvykti (ciltacabtagene autoleucel) and Breyanzi (lisocabtagene maraleucel). These products target CD19 or BCMA, and have been shown to be effective for the treatment of B cell acute lymphoblastic leukaemia (B-ALL), chronic lymphocytic leukaemia (CLL) and non-Hodgkin's lymphoma (NHL) (Grupp et al., 2013, New England Journal of Medicine, 368:1509-1518; Maude et al., 2018, The New England Journal of Medicine, 378 (5): 439-448; Park et al., 2018, The New England Journal of Medicine, 378 (5): 449-459; Porter et al., 2011, The New England Journal of Medicine, 365:725-733).

While CAR T cells have demonstrated efficacy for the treatment of haematological malignancies, this is yet to be achieved for the treatment of solid cancers. In the context of solid tumors, CAR T cells face various barriers that limit their therapeutic efficacy, including tumor heterogeneity, inefficient trafficking into the tumor, and immunosuppressive mechanisms in the tumor microenvironment. Engineering CAR T cells to express immunomodulatory factors, such as pro-inflammatory cytokines, is a strategy that has the potential to overcome multiple limitations of CAR T cell therapy by eliciting widespread effects on endogenous immune mechanisms. While these so-called “armored” CAR T cells have demonstrated promising results in pre-clinical models, their clinical application been limited by severe, life-threatening toxicities including liver dysfunction, high fevers and hemodynamic instability, which may be attributable to constitutive, uncontrolled or unintended transgene expression (Zhang et al., 2015, Clinical Cancer Research, 21:2278-2288).

Accordingly, there remains a need for the development of improved engineered T cells, and methods for their production, which enable their safe and effective use for immunotherapy, e.g., for the treatment of cancer.

### SUMMARY

In an aspect of the present disclosure, there is provided an engineered T cell comprising a heterologous nucleotide sequence encoding at least one immunomodulatory factor, wherein the heterologous nucleotide sequence is introduced in-frame of an endogenous gene of the T cell, wherein expression of the endogenous gene is under the control of an endogenous regulatory element, wherein expression of the heterologous nucleotide sequence is controlled by the endogenous regulatory elements of the endogenous gene, and wherein the endogenous gene is selected from the group consisting of RGS16, RGS1, RGS2, and CLU.

In another aspect of the present disclosure, there is provided an engineered T cell comprising a heterologous nucleotide sequence encoding at least one immunomodulatory factor, wherein the heterologous nucleotide sequence is introduced in-frame of an endogenous gene of the T cell, wherein expression of the endogenous gene is under the control of an endogenous regulatory element, wherein expression of the heterologous nucleotide sequence is controlled by the endogenous regulatory elements of the endogenous gene, and wherein the endogenous gene is RGS16.

In another aspect of the present disclosure, there is provided a pharmaceutical composition comprising the engineered T cell (e.g., a population of the engineered T cells) described herein.

In another aspect of the present disclosure, there is provided a method for the treatment of cancer comprising the administration of a therapeutically effective amount of the engineered T cell (e.g., a population of the engineered T cells), or pharmaceutical composition described herein to a subject in need thereof.

In another aspect of the present disclosure, there is provided a use of the engineered T cell (e.g., a population of the engineered T cells), or pharmaceutical composition described herein in the manufacture of a medicament for the treatment of cancer.

In another aspect of the present disclosure, there is provided a genome editing system comprising:
(a) a sgRNA comprising a sequence of at least 10 contiguous nucleotides that are complementary to a target nucleic acid sequence within an endogenous gene of a T cell, wherein the endogenous gene is selected from the group consisting of RGS16, RGS1, RGS2, and CLU;
(b) a RNA-guided nuclease; and
(c) a homology directed repair (HDR) template, wherein the HDR template comprises a nucleotide sequence encoding at least one immunomodulatory factor.

In another aspect of the present disclosure, there is provided a genome editing system comprising:
(a) a sgRNA comprising a sequence of at least 10 contiguous nucleotides that are complementary to a target nucleic acid sequence within an endogenous gene of a T cell, wherein the endogenous gene is RGS16;
(b) a RNA-guided nuclease; and
(c) a homology directed repair (HDR) template, wherein the HDR template comprises a nucleotide sequence encoding at least one immunomodulatory factor.

In another aspect of the present disclosure, there is provided a method of enhancing the function of a T cell, the method comprising providing to the T cell the genome editing system described herein.

### BRIEF DESCRIPTION OF THE DRAWINGS

Embodiments of the disclosure are described herein, by way of non-limiting example only, with reference to the accompanying drawings.

FIG. 1 shows that knock-in of GFP into the RGS16 target locus (i.e., to achieve transcriptional control of GFP via endogenous RGS16 regulatory elements) via CRISPR/Cas9-mediated homology-directed repair (CRISPR-HDR) in human anti-Lewis Y CAR T cells results in high expression of GFP following T cell stimulation in vitro. (A-B) A graphical representation of GFP expression (mean fluorescence intensity (MFI); y-axis) following CRISPR-HDR editing at different target loci (x-axis) in non-stimulated, anti-CD3/anti-CD28 stimulated, or OVCAR or MCF-7 tumor stimulated human anti-Lewis Y CAR T cells after (A) one stimulation, or (B) three stimulations. Values indicative of mean fluorescence intensity (MFI) of GFP expression in CD8⁺ FLAG (CAR)⁺ T cells.

FIG. 2 shows that knock-in of GFP into the RGS16 target locus via CRISPR-HDR in human anti-Lewis Y CAR T cells results in improved expression of GFP following T cell stimulation in vitro relative to other tumor-selective gene targets, PD-1 and NR4A2. A graphical representation of GFP expression (MFI; y-axis) following CRISPR-HDR editing at different target loci (x-axis) in non-stimulated, or tumor stimulated human anti-Lewis Y CAR T cells. Values indicative of mean fluorescence intensity (MFI) of GFP expression in CD8⁺ FLAG (CAR)⁺ T cells.

FIG. 3 shows that knock-in of GFP into the RGS16, RGS1, RGS2, and CLU target loci via CRISPR-HDR in human anti-Lewis Y CAR T cells results in tumor-selective expression of GFP in vivo. Additionally, it shows that editing of T cells at the RGS16 target locus in a manner designed to conserve endogenous RGS16 expression (GFP-P2A-RGS16) leads to increased tumor selectivity of GFP expression. (A-B) A graphical representation of GFP expression (MFI; y-axis) at (A) day 7, and (B) day 14 post-adoptive transfer in tumors and spleens. Values indicative of mean fluorescence intensity (MFI) of GFP expression in CD8⁺ FLAG (CAR)⁺ T cells. These data are from NSG mice inoculated with 5×10⁶ OVCAR cells, followed by treatment with 2×10⁷ CRISPR/Cas9 edited human anti-Lewis Y CAR T cells at 10-14 days post tumor inoculation.

FIG. 4 shows that knock-in of GFP into the RGS16, RGS1, RGS2, and CLU target loci via CRISPR-HDR in human anti-Lewis Y CAR T cells results in tumor-selective expression of GFP in vivo. A series of representative FACS plots of GFP expression in tumor (top panel) and spleen (bottom panel) at day 14 post-adoptive transfer. These data are from NSG mice inoculated with 5×10⁶ OVCAR-3 cells, followed by treatment with 2×10⁷ CRISPR/Cas9 edited human anti-Lewis Y CAR T cells at 10-14 days post tumor inoculation.

FIG. 5 shows that the proliferation of CRISPR-HDR edited human anti-Lewis Y CAR T cells is not affected by the insertion of a heterologous nucleotide (i.e., GFP) in-frame of RGS16, RGS1, RGS2, and CLU. A graphical representation of Ki67 expression (% Ki67; y-axis) in CD8⁺ FLAG (CAR)⁺ T cells that have been CRISPR-HDR edited at different target loci (x-axis) at (A) day 7, and (B) day 14 post-adoptive transfer. These data are from NSG mice inoculated with 5×10⁶ OVCAR-3 cells, followed by treatment with 2×10⁷ CRISPR/Cas9 edited human anti-Lewis Y CAR T cells at 10-14 days post tumor inoculation.

FIG. 6 shows that the memory phenotype of CRISPR-HDR edited human anti-Lewis Y CAR T cells is not affected by the insertion of a heterologous nucleotide (i.e., GFP) in-frame of RGS16, RGS1, RGS2, and CLU. (A-B) A graphical representation of memory phenotypes of tumor-derived CD8⁺ FLAG (CAR)⁺ T cells (%; x-axis) that have been CRISPR-HDR edited at different target loci (x-axis) at (A) day 7, and (B) day 14 post-adoptive transfer. (C-D) A graphical representation of memory phenotypes of spleen-derived CD8⁺ FLAG (CAR)⁺ T cells (%; x-axis) that have been CRISPR-HDR edited at different target loci (x-axis) at (C) day 7, and (D) day 14 post-adoptive transfer. These data are from NSG mice inoculated with 5×10⁶ OVCAR-3 cells, followed by treatment with 2×10⁷ CRISPR/Cas9 edited human anti-Lewis Y CAR T cells at 10-14 days post tumor inoculation.

FIG. 7 shows that CRISPR-HDR editing at the RGS16 target locus with RGS16 HDR templates that are designed to conserve endogenous RGS16 expression (GFP-P2A-RGS16, RGS16-P2A-GFP) can affect expression of GFP compared to a RGS16 HDR template that disrupts endogenous RGS16 expression (RGS16/GFP). (A-B) A graphical representation of GFP expression (MFI; y-axis) following CRISPR-HDR editing using each of the RGS16 HDR templates (x-axis) in non-stimulated, anti-CD3/anti-CD28 stimulated, or tumor stimulated human anti-Lewis Y CAR T cells after (A) one stimulation, or (B) three stimulations. Values indicative of mean fluorescence intensity (MFI) in CD8⁺ FLAG (CAR)⁺ T cells. RGS16/GFP and GFP-P2A-RGS16 are designed to integrate the transgene adjacent to translational start codon, and RGS16-P2A-GFP is designed to integrate the transgene adjacent to stop codon.

FIG. 8 shows that CRISPR-HDR editing at the RGS16 target locus with RGS16 HDR templates that are designed to conserve endogenous RGS16 expression (GFP-P2A-RGS16, RGS16-P2A-GFP) can affect expression of GFP compared to a RGS16 HDR template that disrupts endogenous RGS16 expression (RGS16/GFP). A series of representative FACS plots of GFP expression in non-stimulated (top panel), or MCF-7 (middle panel) or OVCAR-3 (bottom panel) tumor stimulated human anti-Lewis Y CAR T cells after three stimulations. RGS16/GFP and GFP-P2A-RGS16 are designed to integrate the transgene adjacent to translational start codon, and RGS16-P2A-GFP is designed to integrate the transgene adjacent to stop codon.

FIG. 9 shows that CRISPR-HDR editing of human anti-Lewis Y CAR T cells at the RGS16 target locus with a RGS16 HDR template designed to conserve endogenous RGS16 expression (GFP-P2A-RGS16) improves tumor-selective expression of GFP relative to a RGS16 HDR template that disrupts endogenous RGS16 expression (RGS16/GFP) in vivo. Representative FACS plots of GFP expression in tumor (top panel) and spleen (bottom panel) at day 14 post-adoptive transfer. These data are from NSG mice inoculated with 5×10⁶ OVCAR-3 cells, followed by treatment with 2×10⁷ CRISPR/Cas9 edited human anti-Lewis Y CAR T cells at 10-14 days post tumor inoculation. RGS16/GFP and GFP-P2A-RGS16 are designed to integrate the transgene adjacent to translational start codon.

FIG. 10 shows the efficiency of CRISPR/Cas9-mediated gene knockout of the indicated genes in human anti-Lewis Y CAR T cells. A graphical representation of indel formation (%; y-axis) at different endogenous gene loci (x-axis) based on a comparison of unedited and edited samples using Inference of CRISPR Edits (ICE) analysis (Synthego).

FIG. 11 shows the impact of endogenous gene knockout on cytokine production. A graphical representation of IFNγ production (% or pg/mL; y-axis) in non-stimulated, MCF-7-stimulated or OVCAR-3-stimulated human anti-Lewis Y CAR T cells following CRISPR/Cas9-mediated knockout of endogenous genes (x-axis) measured by (A) intracellular cytokine staining (ICS), or (B) cytometric bead array (CBA).

FIG. 12 shows the impact of endogenous gene knockout on cytokine production. A graphical representation of TNF production (% or pg/mL; y-axis) in non-stimulated, MCF-7-stimulated or OVCAR-3-stimulated human anti-Lewis Y CAR T cells following CRISPR/Cas9-mediated knockout of endogenous genes (x-axis) measured by (A) ICS, or (B) CBA.

FIG. 13 shows the impact of endogenous gene knockout on cytokine production. A graphical representation of IL-2 production (pg/mL; y-axis) in non-stimulated, MCF-7-stimulated or OVCAR-3-stimulated human anti-Lewis Y CAR T cells following CRISPR/Cas9-mediated knockout of endogenous genes (x-axis) measured by CBA.

FIG. 14 shows the impact of endogenous gene knockout on cytotoxicity. A graphical representation of cytotoxicity (% ⁵¹Cr specific release; y-axis) following CRISPR/Cas9-mediated knockout of endogenous genes (x-axis) at an effector to target ratio of 1:1 of CRISPR-KO edited human anti-Lewis Y CAR T cells co-cultured with ⁵¹Cr-labelled MCF-7, or OVCAR-3 tumor cells.

FIG. 15 shows that knock-in of GFP into the RGS16 target locus via CRISPR-HDR in human anti-Lewis Y CAR T cells can be combined with secondary endogenous gene knockout of TNFAIP3 to improve GFP expression following T cell stimulation in vitro. (A-B) A graphical representation of GFP expression (MFI; y-axis) following CRISPR-HDR editing at the RGS16 target locus and CRISPR/Cas9-mediated knockout of the secondary endogenous gene, TNFAIP3 (x-axis) in non-stimulated, or tumor-stimulated human anti-Lewis Y CAR T cells after (A) one stimulation, or (B) three stimulations. Values indicative of mean fluorescence intensity (MFI).

FIG. 16 shows that RGS16 is an optimal target gene for CRISPR-engineered armored T cells. A graphical representation of the difference in GFP MFI between human CD8⁺ CAR T cells harvested from tumor and spleen (AMFI, Tumor-Spleen; y-axis) of mice bearing subcutaneous OVCAR-3 tumors 14 days following adoptive transfer of human anti-Lewis Y CAR T cells engineered to express GFP from the indicated gene loci (x-axis). One-way ANOVA, **** p<0.0001.

FIG. 17 shows that expression of IL-2 via the RGS16 locus in human anti-Lewis Y CAR T cells mediates superior therapeutic efficacy compared to expression via the PD-1 or NR4A2 loci. (A) A graphical representation of tumor area (mm²; y-axis) and time (days post-treatment; x-axis) in mice bearing subcutaneous OVCAR-3 tumors following adoptive transfer of human anti-Lewis Y CAR T cells engineered to express IL-2 from the NR4A2, PDCD1 and RGS16 target loci. Represented as mean±SEM from n=3-6 mice per group. Two-way ANOVA, * p<0.05, ** p<0.01, *** p<0.001. (B) A graphical representation of IL-2 concentration (ng/ml; y-axis) in supernatants of human anti-Lewis Y CAR T cells engineered to express IL-2 from either the NR4A2, PDCD1 or RGS16 gene loci (x-axis) following stimulation with OVCAR-3 or MCF-7 tumor cells for 24 hours. (C) A graphical representation of IL-2 concentration (ng/ml; y-axis) in supernatants of human anti-Lewis Y CAR T cells engineered to express IL-2 from the NR4A2, PDCD1 or RGS16 target loci following stimulation with OVCAR-3 tumor cells for 24 or 72 hours (x-axis). (B-C) Represented as mean±SD of triplicate samples. Two-way ANOVA, * p<0.05, ** p<0.01, **** p<0.0001. (D) A graphical representation of body weight (%; y-axis) of mice bearing subcutaneous OVCAR-3 tumors following adoptive transfer of human anti-Lewis Y CAR T cells engineered to express IL-2 from either the NR4A2, PDCD1 or RGS16 gene loci at up to 8 days post-treatment (x-axis). (E) A graphical representation of serum IL-2 concentrations (pg/mL; y-axis) of mice bearing subcutaneous OVCAR-3 tumors following adoptive transfer of human anti-Lewis Y CAR T cells engineered to express IL-2 from the RGS16 gene locus (x-axis) at 11 days post-treatment. (D-E) Represented as mean±SEM from n=6 mice per group. (F) A graphical representation of Ki67 expression (Percentage Ki67⁺ of CD8⁺ CAR T cells; y-axis) following ex vivo analysis of human CAR T cells isolated from tumors harvested from mice bearing subcutaneous OVCAR-3 tumors following adoptive transfer of human anti-Lewis Y CAR T cells engineered to express IL-2 from RGS16 target locus (x-axis). Represented as mean±SEM from n=4 mice per group. One-way ANOVA, ** p<0.01, *** p<0.001. (G) A graphical representation of the number of human CAR T cells (number of cells/mg tumor or spleen; y-axis) isolated from tumors (left panel) and spleen (right panel) harvested from mice bearing subcutaneous OVCAR-3 tumors following adoptive transfer of human anti-Lewis Y CAR T cells engineered to express IL-2 from RGS16 target locus (x-axis). Represented as mean±SEM from n=4 mice per group. (H) A graphical representation of human CAR T cells (number of cells/mg tumor, spleen or ml of blood; y-axis) isolated from tumors (left panel), spleen (middle panel) and blood (right panel) 14 days post-adoptive transfer of human anti-Lewis Y CAR T cells engineered to express IL-2 from the RGS16 target locus (x-axis). Represented as mean±SEM from n=4-6 mice per group. One-way ANOVA, * p<0.05, *** p<0.001, **** p<0.0001. (I) A graphical representation of the proportion of intratumoral human CD8⁺ CAR T cells expressing 0, 1, 2 or 3 effector proteins (IFNγ, TNF and IL-2). Wheels showing mean proportions from n=4 mice per group. (J) A graphical representation of the proportion of splenic human CD8⁺ CAR T cells with stem cell memory (TSCM), central memory (TCM), effector memory (TEM) or terminally differentiated effector (TEMRA) phenotype based on CD45RA and CD62L expression. Data represented as mean proportions from n=4 mice per group.

FIG. 18 shows the frequency of T cells expressing RGS16 (y-axis) in peripheral blood and tumor of patients across 21 different cancer types (x-axis), analyzed from single-cell RNA sequencing data by Zheng et al.

FIG. 19 shows the clinical applicability of CRISPR-engineered armored CAR T cells. A graphical representation of (A) GFP expression (GFP⁺, %; y-axis) and (B) MFI (y-axis) in human anti-Lewis Y CAR T cells derived from a multiple myeloma patient engineered to express GFP from the RGS16 or NR4A2 target locus following co-culture with OVCAR-3 tumor cells for 72 hours. Represented as mean±SD of triplicate samples.

FIG. 20 shows the clinical applicability of CRISPR-engineered armored CAR T cells. A graphical representation of (A) GFP expression (GFP⁺, %; y-axis) and (B) MFI (y-axis) in human anti-Lewis Y CAR T cells derived from three diffuse large B cell lymphoma patients engineered to express GFP from the RGS16 or NR4A2 target locus following co-culture with OVCAR-3 or MCF-7 tumor cells for 72 hours. Represented as mean±SD of triplicate samples. (C) A graphical representation of IL-2 concentration (pg/mL; y-axis) in supernatants of human anti-Lewis Y CAR T cells derived from a diffuse large B cell lymphoma patient engineered to express IL-2 from the RGS16 target locus following co-culture with OVCAR-3 or MCF-7 tumor cells for 24 hours.

FIG. 21 shows that CRISPR-HDR editing of murine OT-I T cells at the RGS16 target locus to either disrupt (RGS16/GFP) or conserve endogenous RGS16 expression (GFP-P2A-RGS16) can affect expression of GFP both in vitro and in vivo. (A-B) A graphical representation of (A) GFP expression (%; y-axis), or (B) MFI (y-axis) for murine OT-I T cells engineered to express GFP via the RGS16, NR4A2 or PD-1 gene loci (x-axis) following stimulation with plate-bound anti-CD3/28 or AT3 ova tumor cells. (C-D) A graphical representation of (C) GFP expression (% GFP⁺; y-axis) or (D) MFI (y-axis) for murine OT-I T cells engineered to express GFP via the RGS16, NR4A2 or PD-1 gene loci (x-axis) in liver, spleen and tumor assessed 8 days post-adoptive transfer to AT3 ova tumor bearing mice.

FIG. 22 shows that the RGS16 HDR template GFP-P2A-RGS16 conserves endogenous RGS16 expression. A graphical representation of RGS16 mRNA expression (fold change relative to hRpl27; y-axis) where the endogenous RGS16 gene expression was either disrupted (RGS16/GFP) or conserved (GFP-P2A-RGS16) (x-axis).

FIG. 23 shows that knock-in of IL-2 or IL-12 into RGS16 target locus via CRISPR-HDR in murine anti-Her2 CAR T cells results in enhanced cytokine production relative to control (Mock) CAR T cells. A graphical representation of (A) IL-2 or (B) IL-12 concentration (pg/mL; y-axis) where the endogenous RGS16 gene expression was disrupted following the knock-in of IL-2 (RGS16/IL-2) or conserved following the knock-in of IL-12 (IL-12-P2A-RGS16) (x-axis). CAR T cells were stimulated with plate-bound anti-CD3/28, anti-Myc or E0771-Her2 tumor cells for 24 hours before IL-2 or IL-12 secretion was assessed by CBA.

FIG. 24 shows that knock-in of IL-2 or IL-12 into RGS16 target locus via CRISPR-HDR in murine anti-Her2 CAR T cells can affect in vivo tumor growth relative to control (Mock) CAR T cells. (A) A graphical representation of tumor size (mm²; y-axis) and time (days post-treatment; x-axis) where endogenous RGS16 gene expression was disrupted following the knock-in of IL-2 (RGS16/IL-2) (x-axis). (B) A graphical representation of tumor size (mm²; y-axis) and time (days post-treatment; x-axis) where the endogenous RGS16 gene expression was conserved following the knock-in of IL-12 (IL-12-P2A-RGS16) (x-axis).

FIG. 25 shows the ex vivo number and phenotype of peripheral blood murine anti-Her2 CAR T cells CRISPR-engineered to express IL-2 or IL-12 from the RGS16 target locus. (A) A graphical representation of the number of CD8⁺ NGFR (CAR)⁺ T cells (number of cells/mL of blood; y-axis), where endogenous RGS16 gene expression was disrupted following the knock-in of IL-2 (RGS16/IL-2) (x-axis). (B) A graphical representation of the number of CD4⁺ NGFR (CAR)⁺ T cells (number of cells/mL of blood; y-axis), where endogenous RGS16 gene expression was disrupted following the knock-in of IL-2 (RGS16/IL-2) (x-axis). (C) A graphical representation of Ki67 expression (% Ki67⁺ of CD8⁺ NGFR (CAR)⁺ T cells; y-axis), where endogenous RGS16 gene expression was conserved following the knock-in of IL-12 (IL-12-P2A-RGS16) (x-axis).

FIG. 26 shows that CRISPR-HDR editing of human anti-Her2 CAR T cells at the RGS16 target locus with HDR template that is designed to conserve RGS16 expression (GFP-P2A-RGS16) can affect expression of GFP relative to HDR template that disrupts RGS16 expression (RGS16/GFP) following stimulation with plate-bound anti-CD3/28, or PANC-1 or MDA-MB-231 tumor cells (Her2⁺). (A) A graphical representation of GFP expression (%; y-axis) and stimulation conditions (x-axis) for human anti-Her2 CAR T cells engineered to express GFP via the RGS16 gene locus. (B) A graphical representation of GFP expression (MFI; y-axis) and stimulation conditions (x-axis) for human anti-Her2 CAR T cells engineered to express GFP via the RGS16 gene locus.

FIG. 27 shows that CRISPR-HDR editing of human transgenic TCR T cells (targeted against the KRAS G12D neoantigen) at the RGS16 target locus with HDR template that is designed to conserve endogenous RGS16 expression (GFP-P2A-RGS16) can affect expression of GFP relative to HDR template that disrupts RGS16 expression (RGS16/GFP) following stimulation with plate-bound anti-CD3/28, or PANC-1 (KRAS G12D) or MDA-MB-231 (KRAS WT) tumor cells. (A) A graphical representation of GFP expression (%; y-axis) and stimulation conditions (x-axis) for human transgenic TCR T cells engineered to express GFP via the RGS16 gene locus. (B) A graphical representation of GFP expression (MFI; y-axis) and stimulation conditions (x-axis) for human transgenic TCR T cells engineered to express GFP via the RGS16 gene locus.

FIG. 28 shows that human transgenic TCR T cells (targeted against the KRAS G12D neoantigen) edited to express IL-2 at the RGS16 target locus selectively increase cytokine production following stimulation in vitro. (A-B) A graphical representation of IL-2 concentration (pg/mL; y-axis) in the supernatants from TCR T cells stimulated with PANC-1 (KRAS G12D) or MDA-MB-231 (KRAS WT) tumor cells (x-axis) for (A) 24 hours, or (B) 72 hours.

FIG. 29 shows that human anti-Her2 CAR T cells edited to express IL-2 at the RGS16 target locus with HDR templates that either disrupt (RGS16/IL-2) or conserve endogenous RGS16 expression (GFP-P2A-RGS16) increase cytokine production relative to control (Mock) CAR T cells following stimulation in vitro. A graphical representation of IL-2 concentration (ng/ml; y-axis) in the supernatants from human anti-Her2 CAR T cells stimulated with MDA-MB-231 (Her2⁺) tumor cells (x-axis) for 24 hours.

### BRIEF DESCRIPTION OF THE SEQUENCES

Nucleic acid sequences are referred to by sequence identified (SEQ ID NO) with reference to the accompanying Sequence Listing, in which:
SEQ ID NO: 1 is the nucleotide sequence of an sgRNA targeting human RGS16 (for CRISPR-mediated knock-in of transgenes at translational start codon of RGS16).
SEQ ID NO: 2 is the nucleotide sequence of an sgRNA targeting human RGS16 (for CRISPR-mediated knock-in of transgenes at stop codon of RGS16).
SEQ ID NO: 3 is the nucleotide sequence of an sgRNA targeting human RGS16 (for CRISPR/Cas9-mediated knockout).
SEQ ID NO: 4 is the nucleotide sequence of an sgRNA targeting human RGS1 (for CRISPR-mediated knock-in of transgenes at translational start codon of RGS1).
SEQ ID NO: 5 is the nucleotide sequence of an sgRNA targeting human RGS1 (for CRISPR/Cas9-mediated knockout).
SEQ ID NO: 6 is the nucleotide sequence of an sgRNA targeting human RGS2 (for CRISPR-mediated knock-in of transgenes at translational start codon of RGS2).
SEQ ID NO: 7 is the nucleotide sequence of an sgRNA targeting human RGS2 (for CRISPR/Cas9-mediated knockout).
SEQ ID NO: 8 is the nucleotide sequence of an sgRNA targeting human CLU (for CRISPR-mediated knock-in of transgenes at translational start codon of CLU).
SEQ ID NO: 9 is the nucleotide sequence of an sgRNA targeting human CLU (for CRISPR/Cas9-mediated knockout).
SEQ ID NO: 10 is the nucleotide sequence of an sgRNA targeting human PD-1.
SEQ ID NO: 11 is the nucleotide sequence of an sgRNA targeting human TNFAIP3.
SEQ ID NO: 12 is the nucleotide sequence of an sgRNA targeting human NFKBIA.
SEQ ID NO: 13 is the nucleotide sequence of an sgRNA targeting human NR4A2 (for CRISPR-mediated knock-in of transgenes at translational start codon of NR4A2).
SEQ ID NO: 14 is the nucleotide sequence of an sgRNA targeting human NR4A2 (for CRISPR/Cas9-mediated knockout).
SEQ ID NO: 15 is the nucleotide sequence of an sgRNA targeting human SOCS1.
SEQ ID NO: 16 is the nucleotide sequence of a human RGS16/GFP HDR template.
SEQ ID NO: 17 is the nucleotide sequence of a human GFP-P2A-RGS16 HDR template.
SEQ ID NO: 18 is the nucleotide sequence of a human RGS16-P2A-GFP HDR template.
SEQ ID NO: 19 is the nucleotide sequence of a human RGS1/GFP HDR template.
SEQ ID NO: 20 is the nucleotide sequence of a human RGS2/GFP HDR template.
SEQ ID NO: 21 is the nucleotide sequence of a human CLU/GFP HDR template.
SEQ ID NO: 22 is the nucleotide sequence of a human PD-1/GFP HDR template.
SEQ ID NO: 23 is the nucleotide sequence of a human NR4A2/GFP HDR template.
SEQ ID NO: 24 is the nucleotide sequence of a human TNFAIP3/GFP HDR template.
SEQ ID NO: 25 is the nucleotide sequence of a cDNA encoding human NGFR.
SEQ ID NO: 26 is the nucleotide sequence of a cDNA encoding human CXCL9.
SEQ ID NO: 27 is the nucleotide sequence of a cDNA encoding human CXCL10.
SEQ ID NO: 28 is the nucleotide sequence of a cDNA encoding human CXCL9 and CXCL10, separated by a P2A linker.
SEQ ID NO: 29 is the nucleotide sequence of a cDNA encoding human TNF.
SEQ ID NO: 30 is the nucleotide sequence of a cDNA encoding human IFNγ.
SEQ ID NO: 31 is the nucleotide sequence of a cDNA encoding human TNF and IFNγ, separated by a P2A linker.
SEQ ID NO: 32 is the nucleotide sequence of a cDNA encoding human IL-2.
SEQ ID NO: 33 is the nucleotide sequence of a cDNA encoding human IL-12 (IL-12p35p40).
SEQ ID NO: 34 is the nucleotide sequence of a cDNA encoding human IL-12 (IL-12p40p35).
SEQ ID NO: 35 is the nucleotide sequence encoding a P2A linker.
SEQ ID NO: 36 is the nucleotide sequence encoding a T2A linker.
SEQ ID NO: 37 is the nucleotide sequence encoding a poly(A) tail.
SEQ ID NO: 38 is the nucleotide sequence of an sgRNA targeting murine RGS16.
SEQ ID NO: 39 is the nucleotide sequence of a human RGS16/IL-2 HDR template.
SEQ ID NO: 40 is the nucleotide sequence of a human RGS16/IL-12 HDR template.
SEQ ID NO: 41 is the nucleotide sequence of a human IL-2-P2A-RGS16 HDR template.
SEQ ID NO: 42 is the nucleotide sequence of a human IL-12-P2A-RGS16 HDR template.
SEQ ID NO: 43 is the nucleotide sequence of a murine RGS16/IL-2 HDR template.
SEQ ID NO: 44 is the nucleotide sequence of a murine RGS16/IL-12 HDR template.
SEQ ID NO: 45 is the nucleotide sequence of a murine IL-2-P2A-RGS16 HDR template.
SEQ ID NO: 46 is the nucleotide sequence of a murine IL-12-P2A-RGS16 HDR template.
SEQ ID NO: 47 is the nucleotide sequence of a murine RGS16/GFP HDR template.
SEQ ID NO: 48 is the nucleotide sequence of a murine GFP-P2A-RGS16 HDR template.
SEQ ID NO: 49 is the nucleotide sequence of a cDNA encoding murine IL-2.
SEQ ID NO: 50 is the nucleotide sequence of a cDNA encoding murine IL-12.
SEQ ID NO: 51 is the nucleotide sequence of a human RGS16 qRT-PCR forward primer.
SEQ ID NO: 52 is the nucleotide sequence of a human RGS16 qRT-PCR reverse primer.
SEQ ID NO: 53 is the nucleotide sequence of human anti-Her2 CAR (4-1BB-CD3ζ signaling domains).
SEQ ID NO: 54 is the nucleotide sequence of human KRAS G12D TCR.
SEQ ID NO: 55 is the nucleotide sequence of murine anti-Her2 CAR (CD28-CD3ζ signaling domains)

### DETAILED DESCRIPTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. All patents, patent applications, published applications and publications, databases, websites and other published materials referred to throughout the entire disclosure, unless noted otherwise, are incorporated by reference in their entirety. In the event that there is a plurality of definitions for terms, those in this section prevail. Where reference is made to a URL or other such identifier or address, it is understood that such identifiers can change and particular information on the internet can come and go, but equivalent information can be found by searching the internet. Reference to the identifier evidences the availability and public dissemination of such information.

The articles “a”, “an” and “the” include plural aspects unless the context clearly dictates otherwise. Thus, for example, reference to “an allele” includes a single allele, as well as two or more alleles; reference to “a cell” includes a single cell, as well as two or more cells, and so forth.

As used herein, “and/or” refers to and encompasses any and all possible combinations of one or more of the associated listed items, as well as the lack of combinations when interpreted in the alternative (or).

The term “about”, as used herein, means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 10%. Therefore, about 50% means in the range of 45%-55%. Numerical ranges recited herein by endpoints include all numbers and fractions subsumed within that range (e.g., 1 to 5 includes 1, 1.5, 2, 2.75, 3, 3.90, 4, and 5). It is also to be understood that all numbers and fractions thereof are presumed to be modified by the term “about”.

Each embodiment in this specification is to be applied mutatis mutandis to every other embodiment, unless expressly stated otherwise.

Nucleotide and amino acid sequences are referred to by a sequence identifier number (i.e., SEQ ID NO:). The SEQ ID NOs: correspond numerically to the sequence identifiers <400>1 (SEQ ID NO: 1), <400>2 (SEQ ID NO: 2), etc. A sequence listing is provided after the claims. A list describing the SEQ ID NOs in the sequence listing is provided above under the section “Brief Description of the Sequences”.

All sequence identifiers (e.g., GenBank ID, EMBL-Bank ID, DNA Data Bank of Japan (DDBJ) ID, etc.) provided herein were current at the filing date.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements.

The term “optionally” is used herein to mean that the subsequent described feature may or may not be present or that the subsequently described event or circumstance may or may not occur. Hence the specification will be understood to include and encompass embodiments in which the feature is present and embodiments in which the feature is not present, and embodiment in which the event or circumstance occurs as well as embodiments in which it does not.

The present disclosure is predicated, at least in part, on the inventors' surprising finding that engineering T cells by introducing a heterologous nucleotide sequence in-frame within an endogenous gene encoding a tumor-specific factor that is selected from the group consisting of RGS16, RGS1, RGS2, and CLU, provides unexpectedly enhanced tumor-selective expression of the heterologous nucleotide sequence in the T cells upon activation of the cell when compared, for example, to the in-frame insertion of the heterologous nucleotide sequence in another endogenous gene that is selectively expressed at the tumor site, e.g., PDCD1 (i.e., PD1). The engineered T cells of the present disclosure, also referred to as “armored” T cells, are particularly useful for immunotherapy, including where the T cells are engineered by introducing a heterologous nucleotide sequence encoding at least one immunomodulatory factor. For instance, the engineered T cells described herein may be used for the treatment of solid tumors, without the toxicity that has previously been associated with the use of “armored” T cells for the treatment of solid tumors.

Thus, in an aspect disclosed herein, there is provided an engineered T cell comprising a heterologous nucleotide sequence encoding at least one immunomodulatory factor, wherein the heterologous nucleotide sequence is introduced in-frame of an endogenous gene of the T cell, wherein expression of the endogenous gene is under the control of an endogenous regulatory element, wherein expression of the heterologous nucleotide sequence is controlled by the endogenous regulatory elements of the endogenous gene, and wherein the endogenous gene is selected from the group consisting of RGS16, RGS1, RGS2, and CLU.

### Engineered T Cells

In an embodiment, the engineered T cell is derived from a thymocyte, naïve T lymphocyte, immature T lymphocyte, mature T lymphocyte, resting T lymphocyte, activated T lymphocyte, a tumor infiltrating lymphocytes (TILs), helper T cells (i.e., a CD4⁺ T cell), cytotoxic T cell (i.e., a CD8⁺ T cell), a CD4⁺CD8⁺ T cell, a CD4 CD8-T cell, or any other subset of T cells known in the art. The present disclosure also contemplates embodiments in which the engineered T cell is an engineered NKT cell. NKT cells are a specialized subset of lipid-reactive T cells that are involved, directly and indirectly, in immunosurveillance and anti-tumor immunity (see, e.g., Nelson et al., 2021 Cancers (Basel); 13 (20): 5174).

In an embodiment, the T cell is derived from a mammalian donor. In another embodiment, the T cell is derived from a human donor. In an embodiment, the T cell is an NKT cell. In another embodiment, the NKT cell is derived from a human donor.

The term “isolated” as used herein refers to a cell, which is substantially or essentially free from components that normally accompany or interact with it as found in its naturally occurring environment, e.g., whole blood. Methods for the isolation of T cells from whole blood would be known to persons skilled in the art, an illustrative example of which being the Ficoll-Paque method.

The engineered T cells described herein comprise a heterologous nucleotide sequence encoding at least one immunomodulatory factor.

The term “heterologous” as used herein refers to any material introduced from outside of a cell. For example, a nucleotide sequence that is not normally present in a T cell in the copy number, position or configuration. As such, the “heterologous nucleotide sequence” may also be referred to as a “transgene” or “exogenous nucleotide sequence”.

The term “immunomodulatory factor” as used herein refers to any molecule typically expressed by an immune cell (e.g., a macrophage, dendritic cell, T cell, natural killer (NK) cell, B cell) that provides a signal to modulate (e.g., stimulate or inhibit) an immune response. Suitable immunomodulatory factors would be known to persons skilled in the art, illustrative examples of which include cytokines (e.g., TNF, IFNγ, IFNα, IFNβ, IL-2, IL-12, IL-15, IL-18, CXCL9, CXCL10, XCL1 and CD40L), and co-stimulatory molecules (e.g., MHC class I molecule, OX40, CD27, CD28, CDS, ICAM-1, LFA-1 (CDl la/CD18), ICOS (CD278) and 4-1BB (CD137)). The term “immunomodulatory factor” is also intended to encompass synthetic non-naturally occurring immunomodulatory factors, including bi-specific T cell engagers, i.e., BiTEs, bispecific antibodies, dual-affinity retargeting proteins, tandem diabodies), nanobodies (including bi-specific nanobodies), a chimeric antigen receptor (CAR) and adjuvant mRNAs.

In an embodiment, the immunomodulatory factor is a cytokine selected from the group consisting TNF, IFNγ, IFNα, IFNβ, IL-2, IL-12, IL-15, IL-18, CXCL9, CXCL10, XCL1, CD40L, and combinations of the foregoing. In another embodiment, the cytokine is selected from the group consisting of TNF, CXCL9, CXCL10, IFNγ, IL-2, IL-12, and combinations of the foregoing. In another embodiment, the cytokine is IL-2 or IL-12.

In an embodiment, the immunomodulatory factor is a non-naturally occurring factor selected from the group consisting of a bi-specific protein (e.g., bi-specific T cell engagers, i.e., BiTEs, bispecific antibodies, dual-affinity retargeting proteins, tandem diabodies), a nanobody (including bi-specific nanobodies), a CAR, an adjuvant mRNA, and combinations of the foregoing.

As described elsewhere herein, the heterologous nucleotide sequence is introduced in-frame within an endogenous gene of the T cell that encodes a tumor-specific factor, which is under the control of an endogenous regulatory element, wherein expression of the heterologous nucleotide sequence is controlled by the endogenous regulatory element of the endogenous gene. By positioning the heterologous nucleotide sequence in-frame, the expression of the heterologous nucleotide sequence is tumor responsive and/or tumor-selective. Accordingly, unlike previous approaches that have sought to “armor” immune cells to improve immunotherapies (e.g., for the treatment of solid tumors), the expression of the heterologous nucleotide sequence within an endogenous gene encoding a tumor-specific factor is not constitutively expressed and therefore enables the systemic delivery of the engineered T cells (e.g., the adoptive transfer of the engineered T cells) without inducing significant off-target effects, such as toxicity, that may otherwise be associated with constitutive expression of the heterologous nucleotide sequence. The expression of the immunomodulatory factor in a tumor responsive and/or tumor-selective manner thus enables the expression of immunomodulatory factors that, e.g., enhance cytotoxicity, modulate the tumor microenvironment, engage host immunity, improve lymphocyte trafficking and engage DCs to improve the therapeutic efficacy of immunotherapies, such as CAR T cell therapies.

The term “endogenous” as used herein refers to any material from or produced inside a cell. Accordingly, an “endogenous gene” is a gene that is present in the donor T cell (i.e., a wild-type T cell).

In an embodiment, the endogenous gene is RGS1.

In an embodiment, the endogenous gene is RGS2.

In an embodiment, the endogenous gene is CLU.

In an embodiment, the endogenous gene is RGS16.

The term “tumor-selective factor” refers to a gene that is preferentially expressed by T cells in the presence of, or following activation in response to, a tumor (e.g., at the tumor site).

The term “regulatory element” as used herein refers to a nucleotide sequence required for the expression of a gene. In certain embodiments, the regulatory element comprises a promoter. In other embodiments, the regulatory element comprises a promoter, an enhancer and/or other regulatory elements that are required for the expression of a gene.

The term “in-frame” as used herein means that the heterologous nucleotide sequence is introduced within the open reading frame of the endogenous gene. For example, in a position that comprises, or is adjacent to, the translational start codon, or the stop codon of the endogenous gene.

Methods for the integration of heterologous nucleotide sequences would be known to persons skilled in the art, illustrative examples of which include the use of biological agents for producing site-specific DNA breaks, e.g., CRISPR-associated protein endonucleases, endonucleases, ZFNs, meganucleases, etc.

In an embodiment, the heterologous nucleotide sequence is introduced at a position adjacent to the translational start codon of the endogenous gene.

The “translational start codon” is the first codon of an mRNA transcript translated by a ribosome. In an embodiment, the translational start codon of the target gene is ATG. The term “endogenous translational start codon” refers to a translational start codon that originated from the donor T cell (i.e., the wild-type T cell).

In an embodiment, the heterologous nucleotide sequence is introduced at a position no more than about 20 nucleotides upstream or downstream of the translational start codon of the endogenous gene. Accordingly, in an embodiment, the heterologous nucleotide sequence is introduced at the translational start codon of the endogenous gene or about 1, 2, 3, 4, 5, 6, 7, 8, 9, 10, 11, 12, 13, 14, 15, 16, 17, 18, 19, or 20 nucleotides upstream or downstream of the translational start codon of the endogenous gene.

In an embodiment, introduction of the heterologous nucleotide sequence disrupts expression of the endogenous gene.

The term “disrupts” as used herein means a reduction in the level of expression of the endogenous gene. In an embodiment, the level of expression of the endogenous gene is reduced by at least about 40% relative to a T cell without the heterologous nucleotide sequence (i.e., a wild-type T cell). Accordingly, the level of expression of the endogenous gene may be reduced by at least about 40%, at least about 41%, at least about 42%, at least about 43%, at least about 44%, at least about 45%, at least about 46%, at least about 47%, at least about 48%, at least about 49%, at least about 50%, at least about 51%, at least about 52%, at least about 53%, at least about 54%, at least about 55%, at least about 56%, at least about 57%, at least about 58%, at least about 59%, at least about 60%, at least about 61%, at least about 62%, at least about 63%, at least about 64%, at least about 65%, at least about 66%, at least about 67%, at least about 68%, at least about 69%, at least about 70%, at least about 71%, at least about 72%, at least about 73%, at least about 74%, at least about 75%, at least about 76%, at least about 77%, at least about 78%, at least about 79%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99%, or effectively abolished to an undetectable level, i.e., 100%.

In another embodiment, the heterologous nucleotide sequence is introduced at a position adjacent to the stop codon of the endogenous gene.

The term “stop codon” as used herein refers to a codon that signals the end of the nucleotide sequence during translation. Accordingly, termination occurs when a ribosome reaches a stop codon (e.g., UAG, UAA and UGA).

In an embodiment, the heterologous nucleotide sequence is introduced at a position no more than about 20 base pairs upstream or downstream of the stop codon of the endogenous gene. Accordingly, in an embodiment, the heterologous nucleotide sequence is introduced at the stop codon of the endogenous gene or at a position about 1, 2, 3, 4, 5, 6, 7, 8, 9, 10, 11, 12, 13, 14, 15, 16, 17, 18, 19, or 20 nucleotides upstream or downstream of the stop codon of the endogenous gene.

In an embodiment, the heterologous nucleotide sequence further comprises one or more or all of:
(a) a stop codon;
(b) a nucleotide sequence encoding a poly(A) tail; and
(c) a linker.

The term “linker” refers to any nucleotide or group of nucleotides that binds two components of the heterologous nucleotide sequence. Suitable linkers would be known to persons skilled in the art, illustrative examples of which include a P2A linker (e.g., SEQ ID NO: 35) and a T2A linker (e.g., SEQ ID NO: 36).

Linkers may enable the integration of heterologous nucleotide sequences encoding two or more immunomodulatory factors, which are separated from each other to enable the transcription and translation of the two or more immunomodulatory factors. Alternatively, linkers may enable the positioning of the heterologous nucleotide sequence into a position adjacent to the, e.g., stop or start codon of the endogenous gene. The linker can be of any length, but is typically at least 20 nucleotides in length. Accordingly, in an embodiment, the linker is at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, at least 81, at least 82, at least 83, at least 84, at least 85, at least 86, at least 87, at least 88, at least 89, at least 90, at least 91, at least 92, at least 93, at least 94, at least 95, at least 96, at least 97, at least 98, at least 99, at least 100 nucleotides in length, and so on and so forth.

In an embodiment, the linker is selected from a P2A linker and a T2A linker.

A “poly(A) tail” is a polyadenylation signal comprising about 200 adenylate nucleotides at the 3′ end of a heterologous or endogenous mRNA. Suitable nucleotide sequences encoding a poly(A) tail would be known to persons skilled in the art, illustrative examples of which include the nucleotide sequence of SEQ ID NO: 37.

In an embodiment, the heterologous nucleotide sequence comprises a sequence of any one of SEQ ID NOs: 25-34, or a sequence that is at least 80% identical to the sequence of any one of SEQ ID NOs: 25-34. Accordingly, the sequence may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the nucleic acid sequence of SEQ ID NOs: 25-34. Methods for the determination of nucleic acid sequence identity would be known to persons skilled in the art, illustrative examples of which include computer programs that employ algorithms such as BLAST (Altschul et al., 1990, Journal of Molecular Biology, 215 (3): 403-410).

In an embodiment, the engineered T cell further comprises a genetic modification that disrupts the expression, function, or both the expression and function (i.e., knockout), of a second endogenous gene of the T cell, wherein the second endogenous gene is selected from the group consisting of NFKBIA, NR4A2, SOCS1 and TNFAIP3.

In an embodiment, the second endogenous gene is TNFAIP3.

In an embodiment, the engineered T cell further comprises a chimeric antigen receptor (CAR).

The terms “chimeric antigen receptor” or “CAR” as used herein mean a recombinant polypeptide comprising at least an antigen-binding domain that is linked, via hinge and transmembrane domains, to an intracellular signaling domain.

The antigen-binding domain is a functional portion of the CAR that is responsible for transmitting information within the cell to regulate cellular activity via defined signaling pathways. In an embodiment, the antigen-binding domain may comprise an antibody or antibody fragment thereof.

The term “antibody” is used herein in the broadest sense and specifically covers monoclonal antibodies (including full length monoclonal antibodies), polyclonal antibodies, chimeric antibodies, humanized antibodies, human antibodies, multi-specific antibodies (e.g., bispecific antibodies), and single variable domain antibodies as long as they exhibit the desired biological activity. The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (which may be abbreviated as HCVR or V_{H}) and a heavy chain constant region. The heavy chain constant region comprises three domains, C_{H1}, C_{H2} and C_{H3}. Each light chain comprises a light chain variable region (which may be abbreviated as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments disclosed herein, the FRs of an antibody (or antigen-binding portion thereof) may be identical to the human germline sequences, or may be naturally or artificially modified. An amino acid consensus sequence may be defined based on a side-by-side analysis of two or more CDRs. Included within the scope of the term “antibody” is an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known.

An “antigen-binding fragment” may be provided by means of arrangement of one or more CDRs on non-antibody protein scaffolds. “Protein scaffold” as used herein includes but is not limited to an immunoglobulin (Ig) scaffold, for example an IgG scaffold, which may be a four chain or two chain antibody, or which may comprise only the Fc region of an antibody, or which may comprise one or more constant regions from an antibody, which constant regions may be of human or primate origin, or which may be an artificial chimera of human and primate constant regions. The protein scaffold may be an Ig scaffold, for example an IgG, or IgA scaffold. The IgG scaffold may comprise some or all the domains of an antibody (i.e., C_{H1}, C_{H2}, C_{H3}, V_{H}, V_{L}). The antigen-binding fragment may comprise an IgG scaffold selected from IgG1, IgG2, IgG3, IgG4 or IgG4PE. For example, the scaffold may be IgG1. The scaffold may consist of, or comprise, the Fc region of an antibody, or is a part thereof. Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g., monovalent nanobodies, bivalent nanobodies, etc.), small modular immunopharmaceuticals (SMIPs), and shark variable IgNAR domains, are also encompassed within the expression “antigen-binding fragment” as used herein. An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} Or V_{L}-V_{L} dimers. Alternatively, the antigen-binding fragment of an antibody may contain a monomeric V_{H} or V_{L} domain. In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H1}; (ii) V_{H}-C_{H2}; (iii) V_{H}-C_{H3}; (IV) V_{H}-C_{H1}-C_{H2}; (v) V_{H}-C_{H1}-C_{H2}-C_{H3}, (vi) V_{H}-C_{H2}-C_{H3}; (vii) V_{H}-C_{L}; (viii) V_{L}-C_{H1}; (ix) V_{L}-C_{H2}, (x) V_{L}-C_{H3}; (xi) V_{L}-C_{H1}-C_{H2}; (xii) V_{L}-C_{H1}-C_{H2}-C_{H3}; (xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment or antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)). As with full antibody molecules, antigen-binding fragments may be monospecific or multi-specific (e.g., bi-specific). A multi-specific antigen-binding fragment of an antibody will typically comprise at least two different variable domains, wherein each variable domain is capable of specifically binding to a separate antigen or to a different epitope on the same antigen. Any multi-specific antigen-binding molecule format may be adapted for use in the context of an antigen-binding fragment of an antibody of the present invention using routine techniques available in the art.

In an embodiment, the antigen-binding domain comprises an antibody fragment. For example, the antigen-binding domain may comprise a scFv consisting of a V_{L} and V_{H} sequence of a monoclonal antibody (mAb) specific for a tumor cell surface molecule (i.e., tumor antigen).

In an embodiment, the CAR binds an antigen selected from the group consisting of CD19, CD20, CD22, CD30, ROR1, CD123, CD33, CD133, CD138, Claudin 18.2, Claudin 6, BCMA, GD2, Her2, Her1, mesothelin, MUC1, gp100, MART-1, MAGE-A3, MUC16, NY-ESO-1, L1-CAM, CEA, FAP, VEGFR2, WT1, TAG-72, CD171, α-FR, CAIX, PSMA, EGFRvIII CLL-1, GRP78, and Lewis Y. In another embodiment, the CAR binds an antigen selected from Her2 and Lewis Y.

In an embodiment, the CAR comprises at least one signaling domain. In another embodiment, the CAR comprises at least two signaling domains.

Examples of CAR signaling domains include CD3ζ, CD28, 4-1BB, DAP10, OX40, ICOS, DAP12, KIR2DS2, CD3s, CD35, CD3C, CD25, CD27, CD79A, CD79B, CARD11, FcRa, Fcftp, FcRy, Fyn, HVEM, Lck, LAG3, LAT, LRP, NKG2D, NOTCH1, NOTCH2, NOTCH3, NOTCH4, ROR2, Ryk, SLAMF1, Slip76, pTa, TCRa, TCRP, TRIM, Zap70, PTCH2 and LIGHT.

In an embodiment, the CAR comprises a signaling domain selected from the group consisting of the CD28 and CD3ζ signaling domains. In another embodiment, the CAR comprises both the CD28 and CD3ζ signaling domains.

In an embodiment, the engineered T cell further comprises a tumor-specific T cell receptor (TCR).

The terms “T-cell receptor” or “TCR” as used herein mean a recombinant or naturally-occurring heterodimeric polypeptide comprising an alpha polypeptide chain (i.e., alpha chain, a chain) and a beta polypeptide chain (i.e., beta chain, β chain), which is capable of binding to a peptide antigen bound to MHC.

In an embodiment, the TCR binds specifically to an antigen selected from the group consisting of 707-AP, AFP, ART-4, BAGE, Bcr-abl, CAMEL, CAP-1, CASP-8, CDC27m, CDK4/m, CEA, CT, Cyp-B, DAM, EGFRvlll, ELF2M, ETV6-AML1, G250, GAGE, GnT-V, Gp100, HAGE, HER-2/neu, HLA-A, HPV, HSP70-2M, HST-2, hTERT, hTRT, iCE, KIAA0205, KRAS, LAGE (L antigen), LDLR/FUT, MAGE, MART-1/Melan-A, MC1 R, Myosin/m, MUC1, MUM-1, MUM-2, MUM-3, NA88-A, NY-ESO-1, P15, P53, p190 minor, Pml/RARa, PRAME, PSA, PSMA, RAGE, RU1, RU2, SAGE, SART-1, SART-3, SSX1, SSX2, SSX3, SSX4, TEL/AML1, TPI/m, TRP-1, TRP-2, TRP-2/INT2 and WT1.

In an embodiment, the TCR binds specifically to a neoantigen.

The term “neoantigen” as used herein refers to foreign proteins that are absent on normal tissues, but can arise from tumors through various mechanisms, such as mutation (e.g., single nucleotide variants, base insertions and deletions (INDELs), and gene fusions), aberrant transcriptomic variants (e.g., RNA splicing variants, polyadenylation and RNA editing variants, non-coding region variants), post-translational modifications, and integration of viral sequences. Suitable neoantigens would be known to persons skilled in the art, illustrative examples of which include the neoantigens described by Xie et al. (2023, Signal Transduction and Targeted Therapy, 8:9).

In an embodiment, the neoantigen is selected from the group consisting of KRASG12D, KRASG12V, P53R175H, P53Y220C, P53R248, and H3.3K27M.

In an embodiment, the neoantigen is KRAS G12D.

### Pharmaceutical Compositions

In an aspect of the present disclosure, there is provided a pharmaceutical composition comprising the engineered T cell (i.e., a population of the engineered T cells) described herein.

In an embodiment, the pharmaceutical composition comprises a population of the engineered T cells in sufficient number to administer a dosage of 10⁴ to 10⁹ cells/kg body weight. In another embodiment, the pharmaceutical composition comprises a population of the engineered T cells in sufficient number to administer a dosage of 10⁵ to 10⁶ cells/kg body weight, including all integer values within those ranges.

In some embodiments, periodic re-administration of the pharmaceutical composition may be required to achieve a desirable therapeutic effect. The exact amounts and rates of administration of the pharmaceutical composition will depend on a number of factors, examples of which are described elsewhere herein, such as the subject's age, body weight, general health, sex and dietary requirements, as well as any drugs or agents used in combination or coincidental with the administration of the composition. Where multiple divided doses are required, these may be administered hourly, daily, weekly, monthly or at other suitable time intervals or the dose may be proportionally reduced as indicated by the exigencies of the situation. Alternatively, a continuous infusion strategy can be employed.

In an embodiment, the pharmaceutical composition is suitable for parenteral administration. In another embodiment, the composition is suitable for intravenous administration.

The pharmaceutical compositions disclosed herein may be prepared according to conventional methods well known in the pharmaceutical industries, such as those described in Remington's Pharmaceutical Handbook (Mack Publishing Co., NY, USA), comprising a therapeutically effective amount of the composition alone, with one or more pharmaceutically acceptable carriers or diluents.

The term “pharmaceutically acceptable carrier” as used herein means any suitable carriers, diluents or excipients. These include all aqueous and non-aqueous isotonic sterile injection solutions, which may contain anti-oxidants, buffers and solutes to render the composition isotonic with the blood of the intended recipient, aqueous and non-aqueous sterile suspensions, which may include suspending agents and thickening agents, dispersion media, anti-fungal and anti-bacterial agents, isotonic and absorption agents, and the like.

In an embodiment, the pharmaceutical composition further comprises one or more immune adjuvants.

The term “immune adjuvant” as used herein refers to a compound or substance that is capable of enhancing a subject's immune response to the immunogen including, for example, the subject's antibody response to the immunogen. An immune adjuvant may therefore assist to enhance the immune response to an engineered T cell in a subject, compared to the administration of the engineered T cell or in the absence of the immune adjuvant.

Suitable immune adjuvants will be familiar to persons skilled in the art, illustrative examples of which include an inhibitor of the PDL-1: PD-1 axis, a TLR3 agonist, a 4-1BB agonist, a TLR7 agonist, an inhibitor of TIM-3, and an inhibitor of CTLA-4.

It is further contemplated herein that the pharmaceutical composition may be co-administered with one or more other agents suitable for the treatment or amelioration of symptoms associated with cancer, such as a solid tumor, illustrative examples of which include surgery, chemotherapy (e.g., anastrozole, bicalutamide, bleomycin sulfate, busulfan, busulfan injection, capecitabine, N4-pentoxycarbonyl-5-deoxy-5-fluorocytidine, carboplatin, carmustine, chlorambucil, cisplatin, cladribine, cyclophosphamide, cytarabine, cytosine arabinoside, cytarabine liposome injection, dacarbazine, dactinomycin, daunorubicin hydrochloride, daunorubicin citrate liposome injection, dexamethasone, docetaxel, doxorubicin hydrochloride, etoposide, fludarabine phosphate, 5-fluorouracil, flutamide, tezacitibine, gemcitabine, hydroxyurea, idarubicin, ifosfamide, irinotecan, L-asparaginase, leucovorin calcium, melphalan, 6-mercaptopurine, methotrexate, mitoxantrone, mylotarg, paclitaxel, phoenix (Yttrium90/MX-DTPA), pentostatin, polifeprosan 20 with carmustine implant, tamoxifen citrate, teniposide, 6-thioguanine, thiotepa, tirapazamine, topotecan hydrochloride for injection, vinblastine, vincristine, and vinorelbine), radiation, immunosuppressive agents (e.g., cyclosporin, azathioprine, methotrexate, mycophenolate, and FK506), antibodies, or other immunoablative agents (e.g., CAMPATH), targeted agents, steroids, and peptide vaccines.

Such combinations may be administered simultaneous with the pharmaceutical composition or concurrently with the pharmaceutical composition.

### Methods of Treatment and Associated Therapeutic Uses

In an aspect, the present disclosure provides a method for the treatment of cancer comprising the administration of a therapeutically effective amount of the engineered T cell or the pharmaceutical composition disclosed herein to a subject in need thereof.

In another aspect, the present disclosure provides the use of the engineered T cell or the pharmaceutical composition described herein in the manufacture of a medicament for the treatment of cancer.

In an embodiment, the engineered T cell described herein is for use in the treatment of cancer.

The therapeutic regimen for the treatment of cancer can be determined by a person skilled in the art and will typically depend on factors including, but not limited to, the type, size, stage and receptor status of the tumor in addition to the age, weight and general health of the subject. Another determinative factor may be the risk of developing recurrent disease. For instance, for a subject identified as being at high risk or higher risk of developing recurrent disease, a more aggressive therapeutic regimen may be prescribed as compared to a subject who is deemed at a low or lower risk of developing recurrent disease. Similarly, for a subject identified as having a more advanced stage of cancer, for example, stage III or IV disease, a more aggressive therapeutic regimen may be prescribed as compared to a subject that has a less advanced stage of cancer.

The term “cancer” as used herein means any condition associated with aberrant cell proliferation. Such conditions will be known to persons skilled in the art. In an embodiment, the cancer is a primary cancer (e.g., a tumor). In another embodiment, the cancer is a metastatic cancer.

Examples of various cancers are described elsewhere herein and include breast cancer, colorectal cancer, prostate cancer, ovarian cancer, cervical cancer, skin cancer, pancreatic cancer, renal cancer, liver cancer, brain cancer, lymphoma, leukemia, lung cancer, sarcoma and the like. The terms “cancer” and “tumor” may be used interchangeably herein, e.g., encompassing both solid and diffuse or circulating tumors.

In an embodiment, the cancer is a solid cancer.

In an embodiment, the solid cancer is selected from the group consisting of pancreatic cancer, colorectal cancer, breast cancer, melanoma and sarcoma.

In an embodiment, the cancer is a diffuse or circulating tumor.

In an embodiment, the diffuse or circulating tumor is selected from the group consisting of lymphoma, leukemia and multiple myeloma.

In an embodiment, the lymphoma is diffuse large B cell lymphoma.

The term “subject” as used herein refers to any mammal, including livestock and other farm animals (such as cattle, goats, sheep, horses, pigs and chickens), performance animals (such as racehorses), companion animals (such as cats and dogs), laboratory test animals and humans. In an embodiment, the subject is a human. In an embodiment, the subject is an adult. In another embodiment, the subject is a child.

As used herein, the term “effective amount” typically refers to an amount of the engineered T cell or pharmaceutical composition described herein that is sufficient to affect one or more beneficial or desired therapeutic outcomes (e.g., reduction in tumor size). Said beneficial or desired therapeutic outcomes may be measured using clinical techniques known in the art, illustrative examples of which include the measurement of imaging biomarkers, tumor size (e.g., as measured by anatomical imaging modalities, such as CT or MRI), quantification of the presence of inflammatory mediators (e.g., Interleukin-1, TNF, TGF-β, etc.). An “effective amount” can be provided in one or more administrations. The exact amount required may vary depending on factors such as the nature and severity of the cancer to be treated, and the age and general health of the subject.

The terms “treat”, “treating”, “treatment” and the like are used interchangeably herein to mean relieving, reducing, alleviating, ameliorating or otherwise inhibiting the severity and/or progression of cancer, or a symptom thereof, in a subject. It is to be understood that the terms “treat”, “treating”, “treatment” and the like, as used herein, do not imply that a subject is treated until clinical symptoms of cancer have been eliminated or are no longer evident (e.g., elimination of solid tumor mass and associated metastatic lesions, if any). Said treatment may also reduce the severity of cancer by preventing progression or alleviating the symptoms associated with cancer.

The terms “prevent”, “preventing”, “prevention” and the like are used interchangeably herein to mean inhibit, hinder, retard, reduce or otherwise delay the development of cancer and/or progression of cancer, or a symptom thereof, in a subject. In the context of the present disclosure, the term “prevent” and variations thereof does not necessarily imply the complete prevention of the specified event. Rather, the prevention may be to an extent, and/or for a time, sufficient to produce the desired effect. Prevention may be inhibition, retardation, reduction or otherwise hindrance of the event, activity or function. Such preventative effects may be in magnitude and/or be temporal in nature.

In an embodiment, the engineered T cell is an autologous engineered T cell.

The term “autologous” as used herein refers to any material derived from the same subject to whom it is later to be administered into the subject in accordance with the methods disclosed herein. Accordingly, in certain embodiments, T cells isolated from the subject may be contacted with the genome editing systems described herein and cultured ex vivo for a time and under conditions suitable for the integration of the heterologous nucleotide sequence, before being reinfused back into the subject in accordance with the method of treatment described herein.

In another embodiment, the engineered T cell is an allogenic engineered T cell.

The term “allogenic” as used herein refers to any material derived from a different animal of the same species as the subject to whom the material is administered.

### Genome Editing Systems

The present inventors have generated the engineered T cells of the present disclosure by the precise integration of the heterologous nucleotide sequence using CRISPR/Cas9 gene editing. In particular, the use of a CRISPR/Cas9 genome editing system has enabled the integration of a heterologous nucleotide sequence encoding an immunomodulatory factor into the open reading frame of an endogenous gene selected from the group consisting of RGS16, RGS1, RGS2 and CLU under the control of endogenous regulatory elements. As such, the expression of the immunomodulatory factor by the engineered T cells is also controlled by the endogenous regulatory elements of RGS16, RGS1, RGS2 or CLU thereby enabling the tumor-selective expression of the immunomodulatory factor, thus avoiding the toxicity associated with the constitutive expression of immunomodulatory factors that is observed with typical approaches for the production of armored CAR T cells.

Accordingly, in an aspect disclosed herein, there is provided a genome editing system comprising:
(a) a sgRNA comprising a sequence of at least 10 contiguous nucleotides that are complementary to a target nucleic acid sequence within an endogenous gene of a T cell, wherein the endogenous gene is selected from the group consisting of RGS1, RGS2, RGS16 and CLU;
(b) a RNA-guided nuclease; and
(c) a homology directed repair (HDR) template, wherein the HDR template comprises a nucleotide sequence encoding at least one immunomodulatory factor.

The term “genome editing” as used herein refers to a type of genetic alteration in which a heterologous nucleotide sequence is inserted into a target nucleic acid sequence within an endogenous gene of a T cell encoding a tumor-specific factor, i.e., into the genome of a T cell, using one or more RNA-guided nucleases. Any suitable RNA-guided nuclease can be introduced into a T cell to induce genome editing of a target nucleic acid sequence, including CRISPR-associated protein (Cas) endonucleases, zinc finger nucleases (ZFNs), transcription activator-like effector nucleases (TALENs), meganucleases, variants, fragments and combinations thereof. Naturally-occurring and synthetic RNA-guided endonucleases are contemplated herein.

In an embodiment, the genome editing system is a CRISPR/Cas genome editing system. In another embodiment, the genome editing system is a CRISPR/Cas9 genome editing system.

The “clustered regularly interspaced short palindromic repeat” (CRISPR)/“CRISPR-associated protein” (Cas) system (CRISPR/Cas system) evolved in bacteria and archaea as an adaptive immune system to defend against viral attack. The mechanisms of CRISPR-mediated gene editing would be known to persons skilled in the art and have been described, for example, by Doudna et al., (2014, Methods in Enzymology, 546). Briefly, upon exposure to a virus, short segments of viral DNA are integrated in the clustered regularly interspaced short palindromic repeats (i.e., CRISPR) locus. RNA is transcribed from a portion of the CRISPR locus that includes the viral sequence. That RNA, which contains sequence complementarity to the viral genome, mediates targeting of a Cas endonuclease to the sequence in the viral genome. The Cas endonuclease cleaves the viral target sequence to prevent integration or expression of the viral sequence.

CRISPR/Cas genome editing systems may be used to generate a site-specific double strand break (DSB) or single strand break (SSB) within a double-stranded DNA (dsDNA). Once a DSB or SSB is detected in a cell, the DNA repair machinery will repair the break by “non-homologous end-joining” or “NHEJ” or “homology-directed repair” or “HDR”.

NHEJ is triggered to repair double-stranded breaks in which the break ends are directly ligated without the need for a homologous template. Due to the error-prone nature of this repair pathway, small insertions or deletions (INDELs) may be introduced at the target locus near the site of the initial cleavage, and such INDELs can cause frameshift mutations, promote internal ribosomal entry, convert pseudo-mRNAs into protein encoding molecules, or induce exon skipping by disruption of exon splicing enhancers (see, e.g., Tuladhar et al., 2019, Nature Communications, 10:4056). Unpredicted large genome modifications can also be introduced, which can be more than several kilobases (see, e.g., Kosicki et al., 2018, Nature Biotechnology, 36:765-771).

By contrast, HDR accurately and precisely repairs DNA breaks using a homologous template to guide repair. The most common form of HDR is homologous recombination (HR), by which nucleotide sequences are exchanged between two similar or identical molecules of DNA.

As used herein the terms “polynucleotide”, “nucleotide sequence”, “nucleic acid” or “nucleic acid molecule” mean a single- or double-stranded polymer of deoxyribonucleotide, ribonucleotide bases or known analogues or natural nucleotides, or mixtures thereof, and can include molecules comprising coding and non-coding sequences of a gene, sense and antisense sequences and complements, exons, introns, genomic DNA, cDNA, pre-mRNA, mRNA, rRNA, siRNA, miRNA, tRNA, ribozymes, recombinant polypeptides, isolated and purified naturally occurring DNA or RNA sequences, synthetic RNA and DNA sequences, nucleic acid probes, primers and fragments.

The term “target nucleic acid sequence” as used herein refers to a nucleic acid sequence within an endogenous gene of a T cell encoding a tumor-specific factor to which a gRNA is designed to have complementarity, where hybridization between the target nucleic acid sequence and the gRNA promotes the formation of a complex comprising the RNA-guided nuclease, the gRNA and the target nucleic acid sequence (i.e., a genome editing complex).

By “complementary” or “substantially complementary” it is meant that a nucleic acid (e.g., RNA, DNA) comprises a sequence of nucleotides that enables it to non-covalently bind, i.e., form Watson-Crick base pairs and/or G/U base pairs, “anneal”, or “hybridize” to another nucleic acid in a sequence-specific, antiparallel, manner (i.e., a nucleic acid specifically binds to a complementary nucleic acid) under the appropriate in vitro and/or in vivo conditions of temperature and solution ionic strength. Standard Watson-Crick base-pairing includes: adenine/adenosine (A) pairing with thymine/thymidine (T), A pairing with uracil/uridine (U), and guanine/guanosine (G) pairing with cytosine/cytidine (C). In addition, for hybridization between two RNA molecules (e.g., dsRNA), and for hybridization of a DNA molecule with an RNA molecule (e.g., when a target nucleic acid sequence base pairs with a gRNA) G can also base pair with U. For example, G/U base-pairing is partially responsible for the degeneracy (i.e., redundancy) of the genetic code in the context of tRNA anti-codon base pairing with codons in rnRNA. Thus, in the context of this disclosure, a G (e.g., of a target nucleic acid sequence base pairing with a gRNA) is considered complementary to both a U and to C. For example, when a G/U base-pair can be made at a given nucleotide position of a protein binding segment of a guide RNA molecule, the position is not considered to be non-complementary, but is instead considered to be complementary.

Hybridization requires that the two nucleic acids contain complementary sequences, although mismatches between bases are possible. The conditions appropriate for hybridization between two nucleic acids depend on the length of the nucleic acids and the degree of complementarity, variables well known in the art. The greater the degree of complementarity between two nucleotide sequences, the greater the value of the melting temperature (Tm) for hybrids of nucleic acids having those sequences. Typically, the length for a hybridizable nucleic acid is 8 nucleotides or more (e.g., 10 nucleotides or more, 12 nucleotides or more, 15 nucleotides or more, 20 nucleotides or more, 22 nucleotides or more, 25 nucleotides or more, or 30 nucleotides or more).

By “gene” it is meant a unit of inheritance that, when present in its endogenous state, occupies a specific locus on a genome and comprises transcriptional and/or translational regulatory sequences and/or a coding region and/or non-translated sequences (i.e., introns, 5′ and 3′ untranslated sequences).

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode,” “encoding” and the like include an RNA product resulting from transcription of a DNA molecule, a protein resulting from translation of an RNA molecule, a protein resulting from transcription of a DNA molecule to form an RNA product and the subsequent translation of the RNA product, or a protein resulting from transcription of a DNA molecule to provide an RNA product, processing of the RNA product to provide a processed RNA product (e.g., mRNA) and the subsequent translation of the processed RNA product.

The terms “protein”, “peptide” and “polypeptide” are used interchangeably herein to refer to a polymer of amino acid residues linked together by peptide (amide) bonds. The terms refer to a protein, peptide, or polypeptide of any size, structure or function.

### Guide RNA

The terms “guide RNA” or “gRNA” refer to a RNA sequence that is complementary to a target nucleic acid sequence and directs a RNA-guided nuclease to the target nucleic acid sequence. gRNA typically comprises CRISPR RNA (crRNA) and a tracr RNA (tracrRNA). “crRNA” is a 17-20 nucleotide sequence that is complementary to the target nucleic acid sequence, while the “tracrRNA” provides a binding scaffold for the RNA-guided nuclease. crRNA and tracrRNA exist in nature as two separate RNA molecules, which has been adapted for molecular biology techniques using, for example, 2-piece gRNAs such as CRISPR tracer RNAs (cr:tracrRNAs).

The genome editing systems described herein comprise a sgRNA.

The terms “single-guide RNA” or “sgRNA” refer to a single RNA sequence that comprises the crRNA fused to the tracrRNA.

The sgRNA contemplated herein are complementary to a target nucleic acid sequence within an endogenous gene of a T cell encoding a tumor-specific factor under the control of endogenous regulatory elements, wherein the endogenous gene is selected from the group consisting of RGS16, RGS1, RGS2 and CLU.

In an embodiment, the sgRNA comprises a sequence of at least 10 contiguous nucleotides that are complementary to a target nucleic acid sequence. Accordingly, the sgRNA comprises a sequence of at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, or at least 30 nucleotides that are complementary to a target nucleic acid sequence.

In an embodiment, the sgRNA comprises a sequence of at least 20 contiguous nucleotides that are complementary to a target nucleic acid sequence.

Methods and tools for the design of sgRNA would be known to persons skilled in the art, illustrative examples of which include CHOPCHOP, CRISPR Design, sgRNA Designer, Synthego and GT-Scan. Accordingly, sgRNAs complementary to a target nucleic acid in RGS16 could be designed and produced by persons skilled in the art, illustrative examples of which include the sgRNAs described herein, such as the sgRNA shown in Table 1.

In an embodiment, the sgRNA comprises a sequence of SEQ ID NOs: 1-9, or a sequence that is at least 90% identical to the sequence of SEQ ID NOs: 1-9.

### RNA-Guided Nuclease

In an embodiment, the RNA-guided nuclease is a CRISPR-associated (Cas) endonuclease. Suitable Cas endonucleases would be known to persons skilled in the art, illustrative examples of which include Cas3, Cas9, Cas12 (e.g., Cas12a, Cas12b, Cas12c, Cas12d, Cas12e) and Cas14.

In an embodiment, the RNA-guided nuclease is Cas9.

### Homology Directed Repair (HDR) Template

The HDR templates of the genome editing system disclosed herein comprise a nucleotide sequence encoding at least one immunomodulatory factor.

In an embodiment, the HDR template comprises a cDNA sequence encoding at least one immunomodulatory factor.

In an embodiment, the immunomodulatory factor is a cytokine selected from the group consisting of TNF, IFNγ, IFNα, IFNβ, IL-2, IL-12, IL-15, IL-18, CXCL9, CXCL10, XCL1, CD40L, and combinations of the foregoing.

In another embodiment, the cytokine is selected from the group consisting of TNF, CXCL9, CXCL10, IFNγ, IL-2, IL-12, and combinations of the foregoing.

Where combinations of two or more immunomodulatory factors are encoded, the nucleotide sequence encoding each immunomodulatory factor may be separated by a linker.

The structure and arrangement of the HDR template will be determined by reference to the desired position of the alteration to be made to the target nucleic acid sequence. Typically, a HDR template comprises a pair of homology arms corresponding to the target nucleic acid sequence, wherein each pair of homology arms flanks one or more transgenes (i.e., nucleotide sequence encoding at least one immunomodulatory factor). In a particular example, the pair of homology arms comprises a 5′ homology arm and a 3′ homology arm. A “5′ homology arm” refers to a polynucleotide sequence that is identical, or nearly identical, or homologous to a DNA sequence 5′ of a target site (e.g., a target sequence comprising, or adjacent to, the translational start codon or translation stop codon of an endogenous gene). A “3′ homology arm” refers to a polynucleotide sequence that is identical, or nearly identical, or homologous to a DNA sequence 3′ of the target site.

In an embodiment, the target nucleic acid sequence comprises, or is adjacent to, the translational start codon of the endogenous gene.

Introduction of the HDR template adjacent to the translational start codon of the endogenous gene may preserve the expression of the endogenous gene, which may be beneficial where the endogenous gene is beneficial for T cell function or where retention of the endogenous gene helps to maintain the selectivity of transgene expression for the tumor site. Alternatively, introduction of the HDR template adjacent to the translational start codon of an endogenous gene may disrupt the expression of the endogenous gene. This may be desirable where the endogenous gene encodes an immunosuppressive factor.

In an embodiment, the translational start codon of the endogenous gene is within from about 10 nucleotides to about 50 nucleotides upstream or downstream from a protospacer adjacent motif (PAM).

The terms “protospacer adjacent motif” and “PAM” as used herein refer to a nucleotide sequence adjacent to the target nucleic acid sequence. In an embodiment, the PAM is the sequence 5′-NGG-3′, where N is any nucleotide (i.e., A, T, C, G).

In an embodiment, the HDR template further comprises one or more, or all of:
(a) a 5′ homology arm comprising a sequence of from about 250 to about 1000 contiguous nucleotides that are homologous to a region 5′ to the translational start codon of the endogenous gene;
(b) a stop codon;
(c) a nucleotide sequence encoding a poly(A) tail;
(d) a linker; and
(e) a 3′ homology arm comprising a sequence of from about 250 to about 1000 contiguous nucleotides that are homologous to a region 3′ to the translational start codon of the endogenous gene.

In an embodiment, the 5′ homology arm comprises a sequence of from about 250 to about 1000 contiguous nucleotides that are homologous to a region immediately 5′ to the translational start codon of the endogenous gene, preferably about 250, preferably about 260, preferably about 270, preferably about 280, preferably about 290, preferably about 300, preferably about 310, preferably about 320, preferably about 330, preferably about 340, preferably about 350, preferably about 360, preferably about 370, preferably about 380, preferably about 390, preferably about 400, preferably about 410, preferably about 420, preferably about 430, preferably about 440, preferably about 450, preferably about 460, preferably about 470, preferably about 480, preferably about 490, preferably about 500, preferably about 510, preferably about 520, preferably about 530, preferably about 540, preferably about 550, preferably about 560, preferably about 570, preferably about 580, preferably about 590, preferably about 600, preferably about 610, preferably about 620, preferably about 630, preferably about 640, preferably about 650, preferably about 660, preferably about 670, preferably about 680, preferably about 690, preferably about 700, preferably about 710, preferably about 720, preferably about 730, preferably about 740, preferably about 750, preferably about 760, preferably about 770, preferably about 780, preferably about 790, preferably about 800, preferably about 810, preferably about 820, preferably about 830, preferably about 840, preferably about 850, preferably about 860, preferably about 870, preferably about 880, preferably about 890, preferably about 900, preferably about 910, preferably about 920, preferably about 930, preferably about 940, preferably about 950, preferably about 960, preferably about 970, preferably about 980, preferably about 990, or more preferably about 1000 contiguous nucleotides that are homologous to a region immediately 5′ to the translational start codon of the endogenous gene.

In another embodiment, the 5′ homology arm comprises a sequence of from about 350 to about 800 contiguous nucleotides that are homologous to a region 5′ to the translational start codon of the endogenous gene.

In an embodiment, the 3′ homology arm comprises a sequence of from about 250 to about 1000 contiguous nucleotides that are homologous to a region immediately 3′ to the translational start codon of the endogenous gene, preferably about 250, preferably about 260, preferably about 270, preferably about 280, preferably about 290, preferably about 300, preferably about 310, preferably about 320, preferably about 330, preferably about 340, preferably about 350, preferably about 360, preferably about 370, preferably about 380, preferably about 390, preferably about 400, preferably about 410, preferably about 420, preferably about 430, preferably about 440, preferably about 450, preferably about 460, preferably about 470, preferably about 480, preferably about 490, preferably about 500, preferably about 510, preferably about 520, preferably about 530, preferably about 540, preferably about 550, preferably about 560, preferably about 570, preferably about 580, preferably about 590, preferably about 600, preferably about 610, preferably about 620, preferably about 630, preferably about 640, preferably about 650, preferably about 660, preferably about 670, preferably about 680, preferably about 690, preferably about 700, preferably about 710, preferably about 720, preferably about 730, preferably about 740, preferably about 750, preferably about 760, preferably about 770, preferably about 780, preferably about 790, preferably about 800, preferably about 810, preferably about 820, preferably about 830, preferably about 840, preferably about 850, preferably about 860, preferably about 870, preferably about 880, preferably about 890, preferably about 900, preferably about 910, preferably about 920, preferably about 930, preferably about 940, preferably about 950, preferably about 960, preferably about 970, preferably about 980, preferably about 990, or more preferably about 1000 contiguous nucleotides that are homologous to a region immediately 3′ to the translational start codon of the endogenous gene.

In another embodiment, the 3′ homology arm comprises a sequence of from about 350 to about 800 contiguous nucleotides that are homologous to a region 3′ to the translational start codon of the endogenous gene.

In an embodiment, the HDR template comprises a sequence of any one of SEQ ID NOs: 16-21 and 39-42, or a sequence that is at least 80% identical to the sequence of any one of SEQ ID NOs: 16-21 and 39-42. Accordingly, the sequence may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence of any one of SEQ ID NOs: 16-21 and 39-42.

In an embodiment, the HDR template comprises a sequence of any one of SEQ ID NOs: 25-34, or a sequence that is at least 80% identical to the sequence of any one of SEQ ID NOs: 25-34. Accordingly, the sequence may be at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or 100% identical to the sequence of any one of SEQ ID NOs: 25-34.

Accordingly, in an embodiment, the target nucleic acid sequence comprises or is adjacent to the stop codon of the endogenous gene.

Introduction of the HDR template adjacent to the stop codon of the endogenous gene may preserve the expression of the endogenous gene, which may be beneficial where the endogenous gene is beneficial for T cell function, or where retention of endogenous gene expression helps to maintain the selectivity of transgene expression for the tumor site.

In an embodiment, the HDR template further comprises one or more of all of:
(a) a 5′ homology arm comprising a sequence of from about 250 to about 1000 contiguous nucleotides that are homologous to a region 5′ to the stop codon of the endogenous gene;
(b) a linker; and
(c) a 3′ homology arm comprising a sequence of from about 250 to about 1000 contiguous nucleotides that are homologous to a region 3′ to the stop codon of the endogenous gene.

In an embodiment, the 5′ homology arm comprises a sequence of from about 250 to about 1000 contiguous nucleotides that are homologous to a region immediately 5′ to the stop codon of the endogenous gene, preferably about 250, preferably about 260, preferably about 270, preferably about 280, preferably about 290, preferably about 300, preferably about 310, preferably about 320, preferably about 330, preferably about 340, preferably about 350, preferably about 360, preferably about 370, preferably about 380, preferably about 390, preferably about 400, preferably about 410, preferably about 420, preferably about 430, preferably about 440, preferably about 450, preferably about 460, preferably about 470, preferably about 480, preferably about 490, preferably about 500, preferably about 510, preferably about 520, preferably about 530, preferably about 540, preferably about 550, preferably about 560, preferably about 570, preferably about 580, preferably about 590, preferably about 600, preferably about 610, preferably about 620, preferably about 630, preferably about 640, preferably about 650, preferably about 660, preferably about 670, preferably about 680, preferably about 690, preferably about 700, preferably about 710, preferably about 720, preferably about 730, preferably about 740, preferably about 750, preferably about 760, preferably about 770, preferably about 780, preferably about 790, preferably about 800, preferably about 810, preferably about 820, preferably about 830, preferably about 840, preferably about 850, preferably about 860, preferably about 870, preferably about 880, preferably about 890, preferably about 900, preferably about 910, preferably about 920, preferably about 930, preferably about 940, preferably about 950, preferably about 960, preferably about 970, preferably about 980, preferably about 990, or more preferably about 1000 contiguous nucleotides that are homologous to a region immediately 5′ to the stop codon of the endogenous gene.

In another embodiment, the 5′ homology arm comprises a sequence of from about 350 to about 800 contiguous nucleotides that are homologous to a region 5′ to the stop codon of the endogenous gene.

In an embodiment, the 3′ homology arm comprises a sequence of from about 250 to about 1000 contiguous nucleotides that are homologous to a region immediately 3′ to the stop codon of the endogenous gene, preferably about 250, preferably about 260, preferably about 270, preferably about 280, preferably about 290, preferably about 300, preferably about 310, preferably about 320, preferably about 330, preferably about 340, preferably about 350, preferably about 360, preferably about 370, preferably about 380, preferably about 390, preferably about 400, preferably about 410, preferably about 420, preferably about 430, preferably about 440, preferably about 450, preferably about 460, preferably about 470, preferably about 480, preferably about 490, preferably about 500, preferably about 510, preferably about 520, preferably about 530, preferably about 540, preferably about 550, preferably about 560, preferably about 570, preferably about 580, preferably about 590, preferably about 600, preferably about 610, preferably about 620, preferably about 630, preferably about 640, preferably about 650, preferably about 660, preferably about 670, preferably about 680, preferably about 690, preferably about 700, preferably about 710, preferably about 720, preferably about 730, preferably about 740, preferably about 750, preferably about 760, preferably about 770, preferably about 780, preferably about 790, preferably about 800, preferably about 810, preferably about 820, preferably about 830, preferably about 840, preferably about 850, preferably about 860, preferably about 870, preferably about 880, preferably about 890, preferably about 900, preferably about 910, preferably about 920, preferably about 930, preferably about 940, preferably about 950, preferably about 960, preferably about 970, preferably about 980, preferably about 990, or more preferably about 1000 contiguous nucleotides that are homologous to a region immediately 3′ to the stop codon of the endogenous gene.

In another embodiment, the 3′ homology arm comprises a sequence of from about 350 to about 800 contiguous nucleotides that are homologous to a region 3′ to the stop codon of the endogenous gene.

In an embodiment, the HDR template comprises a linker.

As described elsewhere herein, a linker is any nucleotide or group of nucleotides that binds two components of the HDR template. In an embodiment, the linker is selected from a P2A linker (e.g., SEQ ID NO: 35) and a T2A linker (e.g., SEQ ID NO: 36). In a preferred embodiment, the linker is a P2A linker (e.g., SEQ ID NO: 35).

In an embodiment, the configuration of the HDR template is selected from the group consisting of:
(a) 5′-linker-immunomodulatory factor-3′; and
(b) 5′-immunomodulatory factor-linker-3′.

As described elsewhere herein, the present inventors have unexpectedly shown that, by inserting the heterologous nucleotide sequence (i.e., transgene) in-frame in the endogenous gene encoding a tumor-specific factor so as to allow for expression of the endogenous gene upon T cell activation, there is greater expression of the heterologous nucleotide sequence when the engineered T cells is at the site of the tumor in vivo when compared to the level of expression of the heterologous nucleotide sequence when the engineered T cell is distal to the tumor site (e.g., within the spleen). Accordingly, the configuration and components of the HDR template may be suitably adjusted to maintain expression of the endogenous gene where expression of the endogenous gene may be beneficial, e.g., where the endogenous gene is beneficial to T cell function, or where retention of endogenous gene expression helps to maintain the selectivity of transgene expression for the tumor site.

In an embodiment, the heterologous nucleotide sequence (i.e., transgene) is introduced adjacent to the translational start codon of an endogenous gene, wherein expression of the endogenous gene is to be maintained. In an embodiment, the configuration of the HDR template is 5′HA-immunomodulatory factor-linker-3′HA, wherein the 5′HA is the 5′ homology arm, and wherein the 3′HA is the 3′ homology arm. In an embodiment, the configuration comprises 5′HA-immunomodulatory factor/GFP-P2A-3′HA.

In another embodiment, the HDR template comprises the nucleotide sequence of SEQ ID NO: 17, 41 or 42 (i.e., GFP-P2A-RGS16, IL-2-P2A-RGS16, IL-12-P2A-RGS16).

In another embodiment, the heterologous nucleotide sequence (i.e., transgene) is to be introduced adjacent to the stop codon of an endogenous gene, wherein expression of the endogenous gene is to be maintained. In an embodiment, the configuration of the HDR template is 5′HA-linker-immunomodulatory factor-3′HA. In an embodiment, the configuration comprises 5′HA-P2A-immunomodulatory factor/GFP-3′HA. In another embodiment, the HDR template comprises the nucleotide sequence of SEQ ID NO: 18 (i.e., RGS16-P2A-GFP).

Where disruption of the expression of the endogenous gene is beneficial (e.g., to enhance CAR T cell function, or to promote more potent intratumoral expression of the heterologous nucleotide sequence by T cells), the configuration and components of the HDR template may be adjusted to disrupt expression of the endogenous gene, taking into consideration the position where the heterologous nucleotide sequence is to be introduced.

In an embodiment, the HDR template is introduced adjacent to the translational start codon of an endogenous gene and expression of the endogenous gene is to be disrupted. In an embodiment, the configuration of the HDR template is 5′HA-immunomodulatory factor-poly(A) tail-3′HA. In an embodiment, the configuration comprises 5′HA-immunomodulatory factor/GFP-poly(A) tail-3′HA. In another embodiment, the HDR template comprises the nucleotide sequence of SEQ ID NOs: 16, 39 or 40 (i.e., RGS16/GFP, RGS16/IL-2, RGS16/IL-12).

The HDR templates contemplated herein may further comprise one or more additional modifications to, e.g., to prevent sgRNA/Cas9 RNP binding, disrupt expression of the endogenous gene, to introduce or remove a PAM, and/or to remove endogenous restriction enzyme/cleavage sites (e.g., NotI sites).

### Vectors, Delivery Vehicles and Cells

In an embodiment, the HDR template is within a vector.

The vectors can be episomal vectors (i.e., that do not integrate into the genome of a host cell), or can be vectors that integrate into a host cell genome. Vectors may be replication competent or replication-deficient. Exemplary vectors include, but are not limited to, plasmids, cosmids, and viral vectors, such as adeno-associated virus (AAV) vectors, lentiviral, retroviral, adenoviral, herpesviral, parvoviral and hepatitis viral vectors. The choice and design of an appropriate vector is within the ability and discretion of one of ordinary skill in the art. Preferably, however, the vector is suitable for use in biotechnology.

Vectors suitable for use in biotechnology would be known to persons skilled in the art, illustrative examples of which include viral vectors derived from adenovirus, adeno-associated virus (AAV), herpes simplex virus (HSV), retrovirus, lentivirus, self-amplifying single-strand RNA (ssRNA) viruses such as alphavirus (e.g., Semliki Forest virus, Sindbis virus, Venezuelan equine encephalitis, M1), and flavivirus (e.g., Kunjin virus, West Nile virus, Dengue virus), rhabdovirus (e.g., rabies, vesicular stomatitis virus), measles virus, Newcastle Disease virus (NDV) and poxivirus as described by, for example, Lundstrom (2019, Diseases, 6:42).

In an embodiment, the vector is an adeno-associated virus (AAV) vector. Exemplary AAV vectors include, without limitation, those derived from serotypes AAV1, AAV2, AAV3, AAV4, AAV5, AAV6, AAV7, AAV8, AAV9, AAV10, AAV11, AAV12 or AAV13, or using synthetic or modified AAV capsid proteins such as those optimized for efficient in vivo transduction. A recombinant AAV vector describes replication-defective virus that includes an AAV capsid shell encapsidating an AAV genome. Typically, one or more of the wild-type AAV genes have been deleted from the genome in whole or part, preferably the rep and/or cap genes.

In an embodiment, the AAV vector is an AAV5 or AAV6 vector.

Where the HDR template is within a vector, the HDR template may further comprise one or more cut sites for cloning into a vector. For example, SEQ ID NOs: 16-21 and 39-42 are flanked by NotI cut sites at the 5′ and 3′ end (e.g., nucleotides 1-8 and 2010-2017 of SEQ ID NO: 16).

The present disclosure also provides non-viral delivery vehicles of the genome editing systems as described herein, and components thereof. Suitable non-viral delivery vehicles will be known to persons skilled in the art, illustrative examples of which include using lipids, lipid-like materials or polymeric materials, as described by, e.g., Rui et al. (2019, Trends in Biotechnology, 37 (3): 281-293), and nanoparticles/nanocarriers, as described by, e.g., Nguyen et al. (2020, Nature Biotechnology, 38:44-49).

In an embodiment, the sgRNA and the RNA-guided nuclease are complexed as a ribonucleoprotein (RNP).

The terms “ribonucleoproteins”, “RNPs” and “Cas-gRNA ribonucleoproteins” are used interchangeably herein to refer to a complex comprising sgRNA and an RNA-guided nuclease that can be used to directly deliver the sgRNA and RNA-guided nuclease into a T cell.

Suitable methods for the direct delivery of RNPs to cells (e.g., T cells) would be known to persons skilled in the art, illustrative examples of which include microinjection and electroporation. In an embodiment, the RNP is delivered to cells by electroporation.

In an embodiment, the genome editing system further comprises an inhibitor of non-homologous end-joining (NHEJ).

Suitable inhibitors of NHEJ would be known to persons skilled in the art, illustrative examples of which include DNA ligase IV (e.g., SCR7) and DNA-PK inhibitors (e.g., VX-984, NU7026, M3814, AZD-7648).

In an embodiment, the inhibitor of NHEJ is a DNA-PK inhibitor. In another embodiment, the DNA-PK inhibitor is M3814.

In another aspect, the present disclosure provides a cell or a cell extract comprising the genome editing system as described herein.

Cells according to the present disclosure include any cell into which the polynucleotides, vectors and RNPs described herein may be introduced and expressed.

The cell or cell extract contemplated herein may be derived from any species, particularly a vertebrate, and even more particularly a mammal. Suitable vertebrates that fall within the scope of the disclosure include, but are not restricted to, any member of the subphylum Chordata including primates (e.g., humans, monkeys and apes, and includes species of monkeys from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice, rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. In a preferred embodiment, the cell or cell extract is derived from a human.

In an embodiment, the cells are human T cells or murine T cells.

In particular embodiments, the cells are human T cells.

### Methods for Altering a Target Nucleic Acid Sequence

In another aspect, the present disclosure provides a method of altering a nucleic acid molecule in a T cell, the method comprising providing to the T cell the genome editing system as described herein.

The term “altering” as used herein refers to any change to the target nucleic acid sequence, including the insertion of one or more nucleotides (e.g., a heterologous nucleotide sequence) into the target nucleic acid sequence.

The cell may be provided with the genome editing systems described herein using any suitable method known in the art. Such methods include transfection, transduction, viral transduction, microinjection, lipofection, nucleofection, nanoparticle bombardment, transformation, conjugation and the like. The skilled person would readily understand and adapt any such method taking into consideration of whether the components of genome editing system are provided as polynucleotides, vectors or RNPs.

In an embodiment, the sgRNA and RNA-guided nuclease are provided to the T cell complexed as an RNP; and the HDR template is provided to the T cell within a vector.

Those skilled in the art will appreciate that the invention described herein is susceptible to variations and modifications other than those specifically described. It is to be understood that the invention includes all such variations and modifications, which fall within the spirit and scope. The invention also includes all of the steps, features, compositions and compounds referred to or indicated in this specification, individually or collectively, and any and all combinations of any two or more of said steps or features.

Unless otherwise defined, all technical and scientific terms used herein have the same meanings as commonly understood by one of ordinary skill in the art to which this invention belongs.

All patents, patent applications and publications mentioned herein are hereby incorporated by reference in their entireties.

The various embodiments enabled herein are further described by the following non-limiting examples.

### EXAMPLES

### Example 1—Materials and Methods

### Human Anti-Lewis Y CAR T Cell Model

Human PBMCs were activated with anti-CD3 (OKT3, 30 ng/ml) and cultured in RPMI media supplemented with 10% FCS, penicillin/streptomycin, sodium pyruvate, glutamax, NEAA, HEPES, β-mercaptoethanol (50 μM) and IL-2 (600 IU/mL) for 48 hours. T cells were then modified with CRISPR/Cas9 editing prior to transduction.

The PG13 packaging cell line producing retrovirus for anti-Lewis Y CAR were seeded at a density of 1.5×10⁶ cells/T175 in 22 mL of supplemented RPMI three days prior to harvesting viral supernatant. Non-treated 6-well plates (Corning Costar, New York, USA) were coated with 1.5 mL of 15 μg/mL retronectin (Takara, Japan) overnight at 4° C. Plates were washed three times with PBS prior to use. Viral supernatants were filtered through an Acrodisc 0.45 μM filter (Sigma-Aldrich) and 5 mL/well was added to the retronectin-coated plates and centrifuged at 1200×g for 1 hour. CRISPR/Cas9-edited T cells were resuspended at 5×10⁵ cell/mL in 5 mL of fresh viral supernatant containing IL-2 (600 IU/mL) and added to the wells. Plates containing cells were centrifuged at 1200×g for 1 hour and incubated overnight at 37° C. This process was repeated the next day. Following transduction, cells were cultured at a density of 2×10⁶ cells/mL in RPMI supplemented with 10% FCS, penicillin/streptomycin, sodium pyruvate, glutamax, NEAA, HEPES, β-mercaptoethanol and 600 IU/mL IL-2. Transduced cells were rested and the expression of CAR was assessed via flow cytometry 3-7 days after the transduction process.

### Lentiviral Transduction of Human T Cells

human anti-Her2 CAR T cells and KRASG12D TCR T cells were generated by lentiviral transduction. Lentiviral packaging plasmids (pCMV-VSV-G, pMDLg/pRRE, pRSV-Rev) and plasmid vectors encoding a second-generation anti-Her2 CAR (SEQ ID NO: 53) or the KRAS G12D TCR (SEQ ID NO: 54) were used. In brief, packaging plasmids and transgene plasmids were transfected into HEK293T cells. Across the following 3 days, cell culture supernatants were collected, pooled and centrifuged with Lenti-X-Concentrator (Takara Bio) to concentrate lentivirus. Lentivirus was used to transduce human T cells activated with OKT3 (30 ng/ml) and IL-2 (600 IU/mL) for 48 hours by adding virus directly to cell cultures at a multiplicity of infection of 0.5 in Lentiboost (Sirion).

### Murine CAR T Cell Model

Retroviral supernatants were harvested from the GP+e86 packaging line for transduction of murine T cells with an anti-hHer2 CAR (SEQ ID NO: 55). Following transduction, murine T cells were maintained in media containing hIL-2 (100 IU/mL) and mIL-7 (200 μg/mL) for in vitro assays or mIL-7 (200 μg/mL) and hIL-15 (10 ng/ml) for in vivo applications.

### CRISPR/Cas9 Gene Editing

Per 1×10⁶ activated human T cells to be electroporated, 26.6 pmoles recombinant Cas9 (IDT) and 270 pmoles sgRNA (Synthego) were combined and incubated for 10 min at RT to generate Cas9/sgRNA ribonucleoprotein (RNP) complexes.

Human T cells (1×10⁶) were resuspended in 20 μL P3 Buffer (Lonza), or TheraPEAK X-VIVO 15 (Lonza) combined with RNP and electroporated using a 4D-Nucleofector (Lonza) using pulse code EO115. Pre-warmed complete RPMI media was then immediately added to electroporation wells and T cells were transferred to a separate vessel for incubation with AAV6 (Packgene or Vigene) encoding CRISPR-HDR templates of interest for 4 hours at 37° C.

Murine T cells were activated from naïve splenocytes by culturing in complete RPMI containing β-mercaptoethanol (50 μM), murine anti-CD3 (0.5 mg/mL), murine anti-CD28 (0.5 mg/mL), hIL-2 (100 IU/mL) and mIL-7 (200 μg/mL) for 24 hours. To perform CRISPR/Cas9 editing, 26.6 pmoles recombinant Cas9 (IDT) and 270 pmoles sgRNA (Synthego) were combined and incubated at RT for 10 minutes to form Cas9/sgRNA RNP complexes. 20×10⁶ murine T cells were resuspended in 20 μL P3 electroporation buffer (containing 82% P3 buffer and 18% Supplement 1; Lonza), combined with RNP and electroporated using a 4D-Nucleofector X Unit (Lonza) using pulse code CM137. For CRISPR-mediated knock-out, 100 μl of prewarmed media was immediately added, and T cells were incubated for 10 minutes before being transferred to an appropriate culture plate. For CRISPR-mediated knock-in, T cells were immediately added to AAV6 at a multiplicity of infection (MOI) of 10-100K and incubated for 4 hours before AAV6 was washed off and downstream protocols were performed.

### Flow Cytometry

For immunofluorescence staining, morphology of cells was determined via FSC-A and SSC-A, doublets were excluded via FSC-A and FSC-H and dead cells excluded via Fixable-Yellow live/dead stain (Invitrogen, Carlsbad, CA, USA).

Extracellular staining: Cell suspensions in round-bottom 96-well plates were centrifuged at 1400 rpm for 4 minutes and supernatants were discarded. Cells were stained with a cocktail of fluorochrome-conjugated antibodies in a total volume of 30-50 μL/well for 30 minutes at 4° C. Cells were washed twice with 100 μL of flow cytometry buffer.

Intracellular staining: Surface stained cells that required intracellular staining were fixed and permeabilized using a Cytofix/Cytoperm Solution Kit according to the manufacturer's instructions (BD Pharmigen). Cells were washed twice in 200 μL of fixation/permeabilization buffer and centrifuged at 1400 rpm for 4 minutes and supernatants were discarded. Cells were stained with a cocktail of fluorochrome-conjugated antibodies in a total volume of 30-50 μL/well for 30 minutes at room temperature in the dark. Cells were washed again, and resuspended in 100 μL of fixation/permeabilization buffer for flow cytometry analysis.

Data was collected using a BD CANTO, BD Fortessa, or BD Symphony (BD Biosciences, San Jose, California, USA) and analysed using FacsDiva v8 and Flowlogic v10.2 software (Miltenyi Biotec, Auburn, California, USA).

### Cytokine Production

Cytokine production of human or murine CAR T cells following in vitro stimulation was determined by Intracellular cytokine staining (ICS) or Cytometric bead array (CBA). ICS was used to measure IFNγ and TNF expression levels. CBA was also performed utilizing supernatants obtained from the in vitro co-culture/stimulation assays. Briefly, BD capture beads for IFNγ, TNF, IL-2 and IL-12 were incubated for 1 hour at room temperature, followed by a 1 hour room temperature incubation of the PE detection beads. Following this, samples were analysed using the FACS Verse, where output was displayed as pg/mL, as determined from the standard curve for each bead.

### Chromium Release Assay

Target tumor cells were labelled with 100 μCi chromium-51 (⁵¹Cr) for 1 hour at 37° C. with 5% CO₂. Tumor cells were washed three times with serum-free RPMI media and centrifuged at 1500 rpm for 4 minutes. Tumors were re-suspended in supplemented RPMI media and plated at 1×10⁴ cells/well in a V-bottom 96-well plate. T cells were added to tumor cells at indicated effector cell:target ratios (E:T ratios). Control wells containing ⁵¹Cr-labelled tumors cells alone or tumor cells with 100 μL of 10% Triton X were included to determine spontaneous and total chromium release, respectively. Plates were incubated overnight at 37° C. with 5% CO₂. Following incubation, plates were centrifuged at 1500 rpm for 5 minutes and 100 μl of supernatant from each well was transferred to 1.2 mL microtiter tubes (Quality Scientific Plastics, San Diego, California, USA). Chromium release was measured using an automatic gamma counter Wallac Wizard 1470 (General Electricity Healthcare, Amersham Australia, KewDale, Western Australia).

Percentage of tumor cell lysis was calculated using the formula: [(sample counts per minute-spontaneous counts per minute)/(total counts per minute-spontaneous counts per minute)]×100%.

### In Vitro Co-Culture/Stimulation Assay

Murine and human T cells were co-cultured with tumor cells (E0771-Her2 for murine anti-Her2 CAR T cells, MCF-7 (Lewis Y⁺) and OVCAR (Lewis Y⁺) for human anti-Lewis Y CAR T cells, MDA-MB-231 (Her2⁺) and PANC-1 (Her2⁺) for human anti-Her2 CAR T cells, PANC-1 for KRAS G12D TCR T cells and AT3-ova for OT-I T cells) at an effector to target (E:T) ratio of 1:1 for 24 hours prior to collection of supernatants and flow cytometry analysis of T cells. For 72 hour chronic stimulation assays, supernatant was completely removed at the 24 hour and 48 hour timepoints and equivalent number of fresh tumor cells were added. For T cell stimulation with anti-CD3 (0.5 mg/mL), anti-CD28 (0.5 mg/mL) or anti-Myc tag antibody (1:1000), a U-bottom 96-well plate was coated with 100 mL PBS containing the appropriate dilutions of antibody at 37° C. for 2 hours before wells were washed twice with 200 mL PBS and T cells were added.

### Adoptive Transfer Experiments

For murine adoptive transfer experiments, hHer2 transgenic mice or Ly5.1 mice were inoculated with 2×10⁵ E0771-hHer2 or 5×10⁵ AT-3-ova in the fourth mammary fat pad. Tumors were allowed to establish for 6 days (E0771-Her2) or 8 days (AT-3-ova) before mice were preconditioned with 4 Gy (E0771-Her2) or 0.5 Gy (AT-3-ova) total body X-ray irradiation (TBI). For human adoptive transfer experiments, NSG mice were inoculated with 5-6×10⁶ OVCAR-3 subcutaneously. Tumors were allowed to establish for 10-16 days (OVCAR-3) prior to preconditioning with 1 Gy TBI. Mice were intravenously injected with 1 dose of 0.5×10⁷ OT-I T cells or 2 doses of 0.25-1×10⁷ murine or human CAR T cells on consecutive days, and intraperitoneally injected with 5 doses of hIL-2 (25,000 IU/dose) on consecutive days.

For tumor growth experiments, tumor area was measured using callipers every 2-4 days until all mice reached ethical endpoint. The ethical endpoint for tumor size was 150 mm². For ex vivo flow cytometry analyses of the tumor and spleen, tissues were harvested on days 7-9 for murine CAR T cell experiments, and days 7 or 14 for human CAR T cell experiments unless indicated otherwise. Tumors were processed by mechanical dissociation followed by enzymatic digestion with serum-free DMEM containing 1 mg/mL collagenase type IV (Sigma-Aldrich) and 0.02 mg/mL DNAse I (Sigma-Aldrich) at 37° C. for 30 minutes with gentle shaking, then filtered through a 70 μm filter followed by a 35 μm filter prior to staining. Spleens were processed by macerating and filtering through a 70 umm filter, red blood cells were lysed with ACK lysis buffer, then samples were filtered through a 35 μm filter prior to staining. For experiments requiring ex vivo stimulation, samples were stimulated with 10 ng/mL phorbol 12-myristate 13-acetate (PMA) and 1 mg/mL ionomycin, in addition to GolgiPlug (BD Biosciences) and GolgiStop (BD Biosciences) at a 1:1000 and 1:1500 dilution, respectively. Samples were incubated at 37° C. for 3 hours prior to staining.

For flow cytometry and serum analyses of blood, 200 mL blood was collected via either retro-orbital or submandibular bleeds into Eppendorf tubes containing 10 μL EDTA. For flow cytometry analysis, red blood cells were lysed using 75 mL ACK lysis buffer prior to staining. For serum analyses, blood was centrifuged at 10,000×g at 4° C. for 15-20 minutes and serum was collected for downstream analyses.

### Toxicity Analysis

The safety of engineered T cells in mice was assessed through various parameters, including the monitoring of weight and physical changes during treatment, as well as the serum analyses of cytokine levels.

### Single-Cell RNA Sequencing Analysis

A scRNA-seq atlas of publicly available and previously published datasets of tumor infiltrating, healthy tissue and peripheral blood T cells from 21 different cancer types was analyzed as per the methods of Zheng et al. (2021, Science, 374). Data integration was performed by per-cell size-factor normalization and per-gene z-score scaling across cells for each dataset. Each dataset was then partitioned into mini-clusters to reduce noise before batch-correction with the Harmony package (Korsunsky et al., 2019, Nature Methods, 16:1289-1296). Seurat was then used to further cluster integrated datasets into ‘meta-clusters’. To determine binarized expression of genes of interest across different cancer types above mean expression, the scale and average.cell functions from the sscVis package was used, and total frequency of cells from each scRNA-seq dataset was plotted as a box-plot.

### Example 2—sgRNA and HDR Template Design

### Disruption of Target Gene Expression

To design sgRNAs suitable for CRISPR/Cas9-mediated knock-in at a site within RGS16, Cas9 5′-NGG-3′ protospacer adjacent motif (PAM) were identified within 20 base pairs of the translational start codon of the gene (where “N” is any A, T, G, or C nucleotide). Guide sequences were then designed to comprise a 20 bp RNA sequence complementary to the DNA sequence 5′ of the PAM site. Guide sequences were triaged according to their on-target and off-target scores calculated as described by Doench et al. (2016, Nature Biotechnology, 34:184-191). To further interrogate possible off-target editing effects, guide sequences were assessed through the COSMID tool (Cradick et al., 2014, Molecular Therapy—Nucleic Acids, 3 (12) e214) and any predicted off-target sites considered using the UCSC genome browser (Kent et al., 2002, Genome Research, 12 (6): 996-1006) to ensure that potential off-target sequences were not in coding regions of genes.

Exemplary sgRNAs targeting human RGS16, RGS1, RGS2 and CLU are shown in Table 1.

Following this guide design, homology directed repair (HDR) templates for disruption of the expression of the endogenous gene were constructed comprising the following components, from 5′ to 3′:
(a) a 350-750 bp 5′ homology arm, homologous to the 350-750 bp genomic DNA region immediately 5′ to the translational start codon of the endogenous gene;
(b) the cDNA sequence of the transgene to be inserted (e.g., GFP, exogenous immunomodulatory factor), provided in frame with the endogenous gene expressing a tumor-specific factor (e.g., RGS16, RGS1, RGS2, CLU, PD-1);
(c) a stop codon;
(d) a sequence coding for a poly(A) tail; and
(e) a 350-750 bp 3′ homology arm, homologous to the 350-750 bp genomic DNA region immediately 3′ to the translational start codon of the endogenous gene.

Exemplary HDR templates are shown in Table 2 (see, e.g., RGS16/GFP).

### Conservation of Target Gene Expression

Where the endogenous target gene is beneficial to CAR T cell function or where retention of the endogenous gene helps to maintain the selectivity of transgene expression for the tumor site, HDR templates were designed to preserve expression of the endogenous gene. Accordingly, the heterologous nucleotide sequence encoding at least one immunomodulatory factor is upstream of a P2A or T2A linker sequence (i.e., 5′-GFP/exogenous immunomodulatory factor-linker-3′). In this variation, HDR templates for preservation of endogenous gene expression were constructed comprising the following components, from 5′ to 3′:
(a) a 350-750 bp 5′ homology arm, homologous to the 350-750 bp genomic DNA region immediately 5′ to the translation start site of the endogenous gene;
(b) the cDNA sequence of the transgene to be inserted (e.g., GFP, exogenous immunomodulatory factor), provided in-frame with the endogenous gene expressing a tumor-specific factor (e.g., RGS16, RGS1, RGS2 or CLU);
(c) a P2A or T2A linker sequence, provided in-frame of the endogenous gene; and
(d) a 350-750 bp 3′ homology arm, homologous to the 350-750 bp genomic DNA region immediately 3′ to the translation start site of the endogenous gene.

Where the HDR template is for insertion of transgene at a position adjacent to, or comprising the stop codon of the endogenous gene, HDR templates for the preservation of endogenous gene expression were constructed comprising the following components, from 5′ to 3′:
(a) a 350-750 bp 5′ homology arm, homologous to the 350-750 bp genomic DNA region immediately 5′ to the stop codon of the endogenous gene;
(b) a P2A or T2A linker sequence, provided in-frame of the endogenous gene;
(c) the cDNA sequence of the transgene to be inserted (e.g., GFP, exogenous immunomodulatory factor), provided in-frame with the endogenous gene expressing a tumor-specific factor (e.g., RGS16, RGS1, RGS2 or CLU); and
(d) a 350-750 bp 3′ homology arm, homologous to the 350-750 bp genomic DNA region immediately 3′ to the stop codon of the endogenous gene.

### Example 3—In Vitro Assessment of CRISPR HDR Edited T Cells

Human anti-Lewis Y CAR T cells were engineered by CRISPR-HDR to express GFP downstream of indicated promoters by electroporating human T cells in the presence of a Cas9/RGS16 sgRNA RNP, Cas9/CLU sgRNA RNP, Cas9/NR4A2 sgRNA RNP, Cas9/RGS1 sgRNA RNP, Cas9/RGS2 sgRNA RNP and Cas9/TNFAIP3 sgRNA RNP. Following electroporation, cells were incubated with AAV6 encoding the relevant CRISPR-HDR templates for 4 hours at 37° C. before they were transduced to express the CAR.

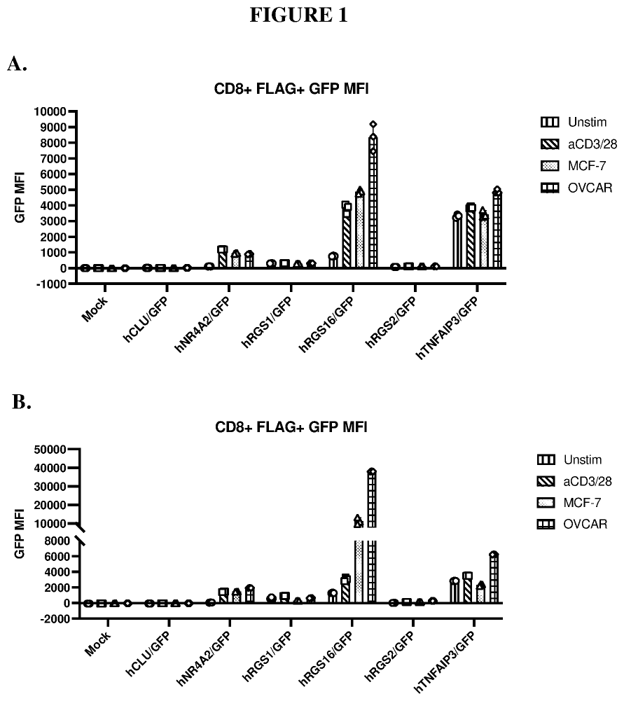

For primary human T cells, peripheral blood mononuclear cells (PBMCs) were activated with soluble anti-CD3 and IL-2 for 48 hours before electroporation, as described above. Edited T cells were cultured for 72 hours before they were stimulated with plate-bound anti-CD3+anti-CD28 or the indicated tumor cell lines (MCF-7 or OVCAR-3) for 24 or 72 hours to upregulate target gene expression, then GFP expression by CD8⁺ CAR T cells was analyzed by flow cytometry (FIG. 1; Table 5).

Successful editing was observed for all target genes, as compared to unstimulated control cells and cells electroporated with Cas9 only (“Mock”) (FIG. 1).

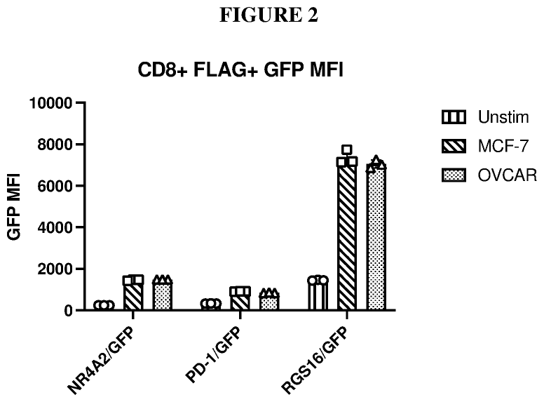

RGS16-edited cells also showed improved performance relative to PD-1- and NR4A2-edited cells following in vitro T cell stimulation (FIG. 2).

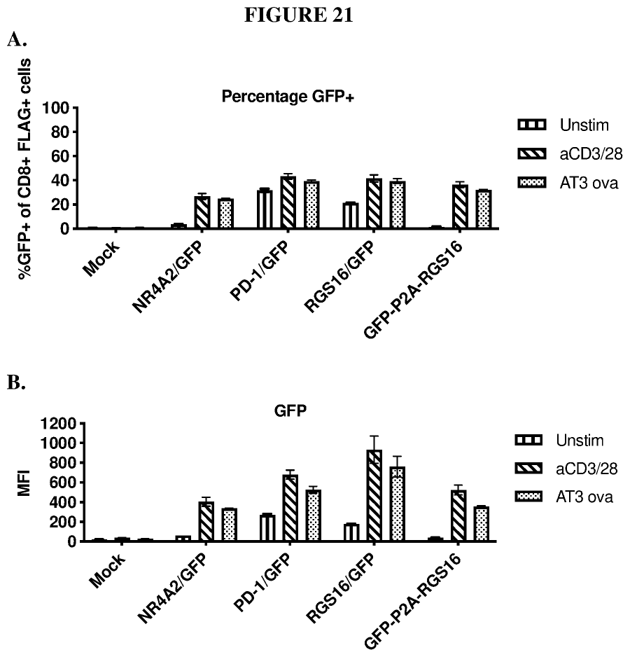

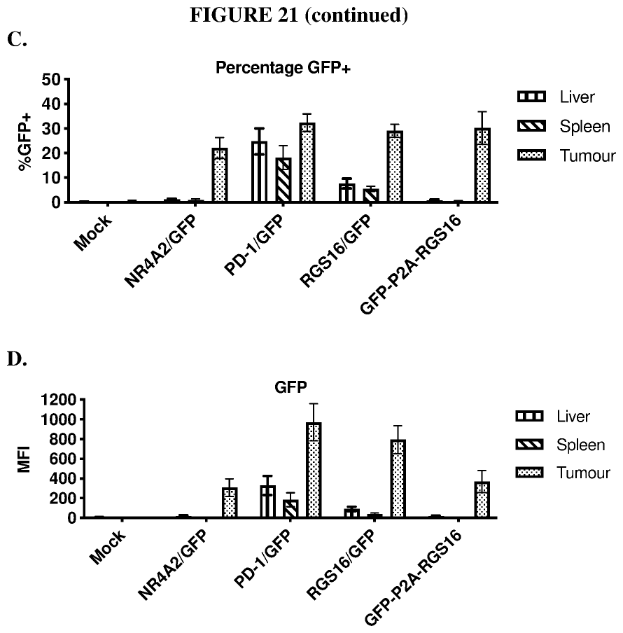

Similarly, for murine OT-I T cells edited to express GFP via the NR4A2, PD-1 or RGS16 gene loci, cells edited to express GFP at the RGS16 gene locus showed improved performance relative to cells edited at the NR4A2 gene locus following stimulation with both anti-CD3/28 and AT3 ova tumor cells, and improved performance relative to cells edited at the PD-1 gene locus following stimulation with AT3 ova tumor cells (Table 6; FIG. 21).

### Example 4—In Vivo Assessment of CRISPR HDR Edited T Cells

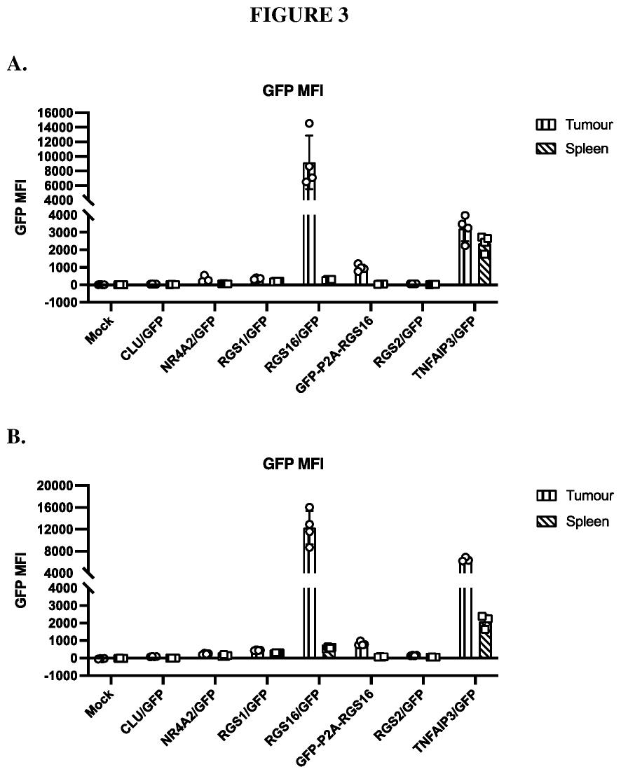

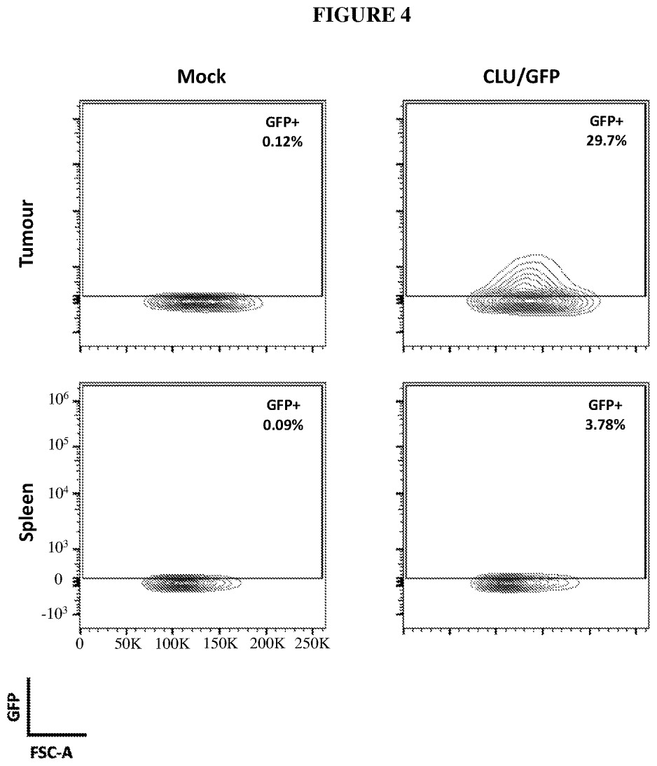

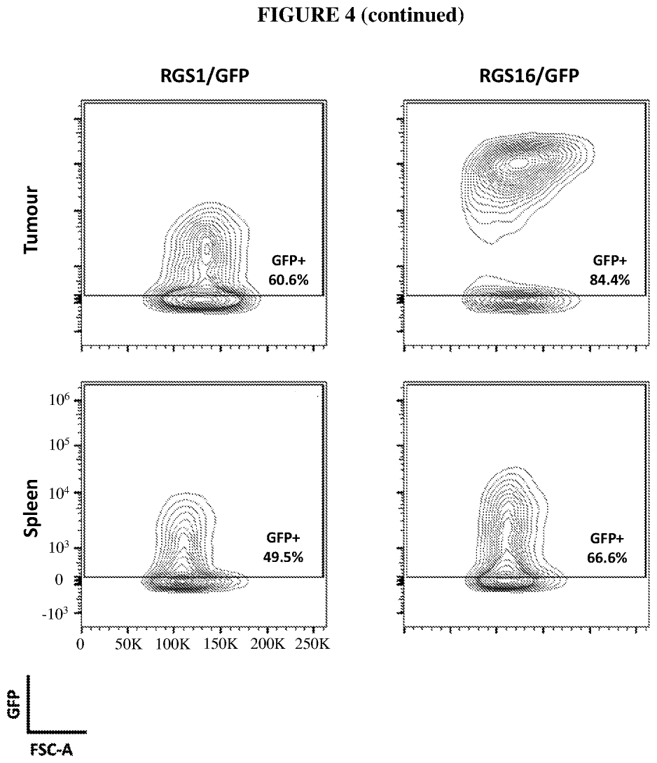

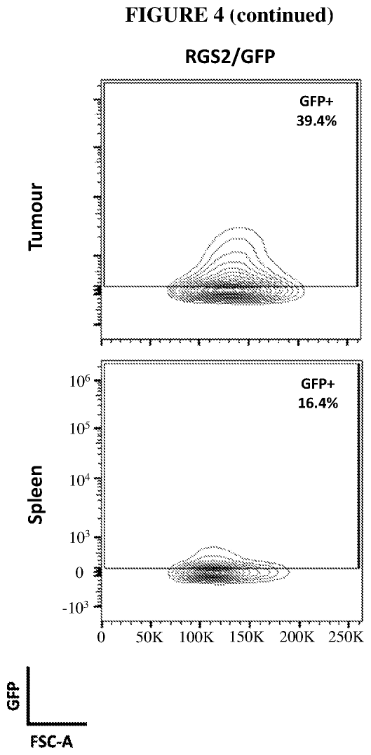

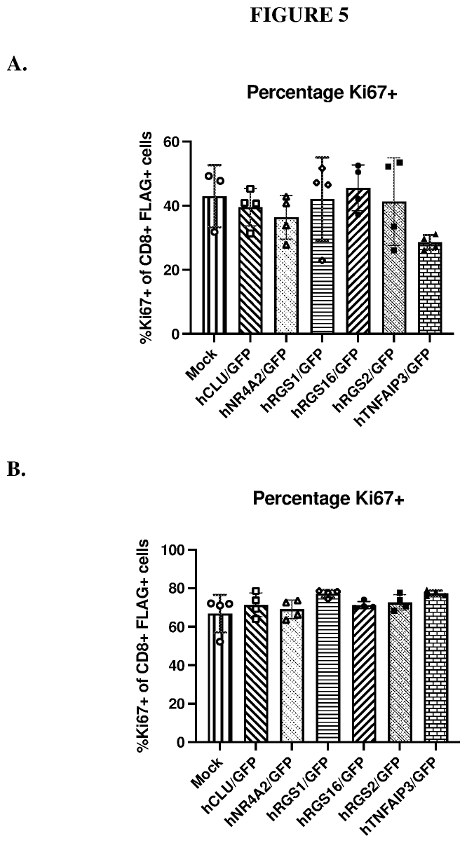

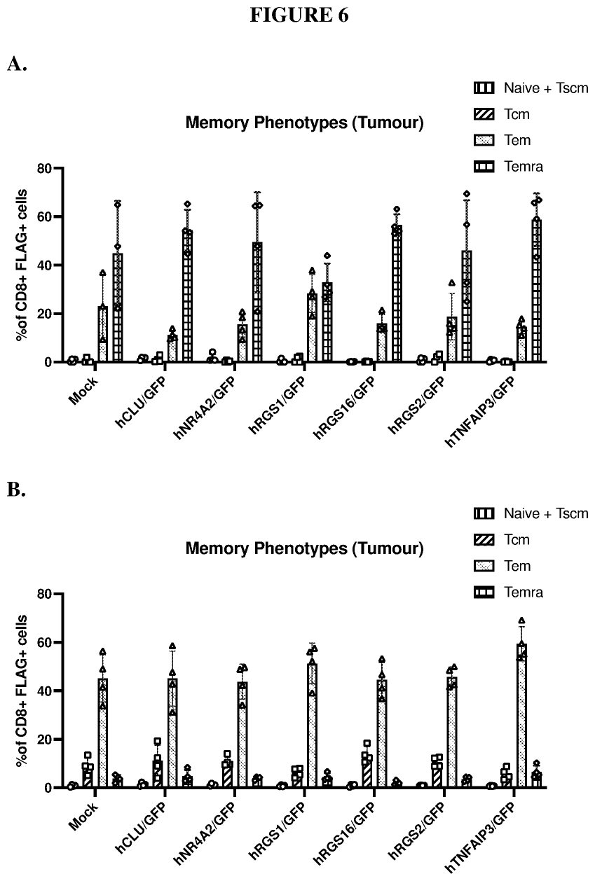

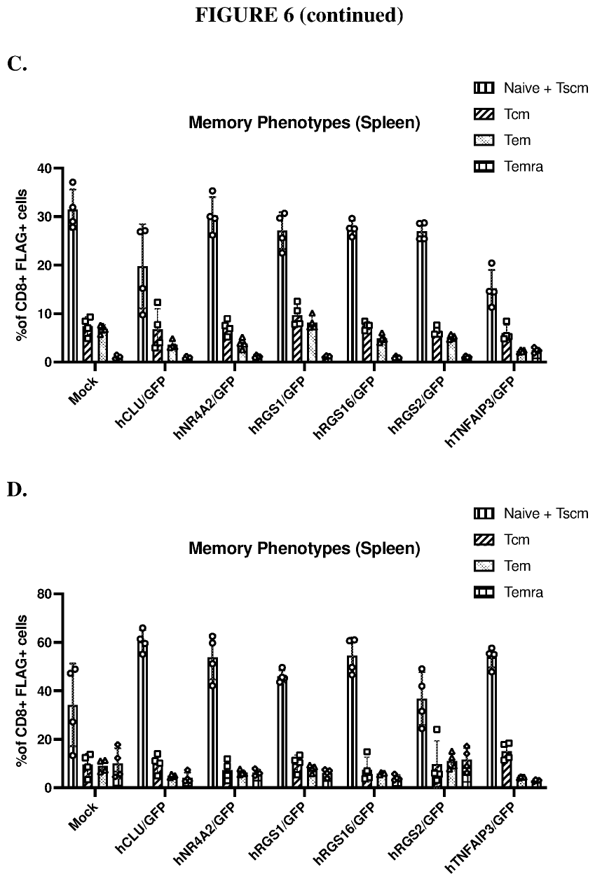

CRISPR-HDR edited human anti-Lewis Y CAR T cells were injected into mice bearing established Lewis Y⁺ OVCAR tumors (20-30 mm²) following 1Gy total body irradiation. Mice were treated with 25,000 units of IL-2 per day for days 0-4, and at 7 and 14 days post treatment, the expression of GFP by CD8⁺ FLAG (CAR)⁺ T cells isolated from spleens or tumors was assessed by flow cytometry. As shown in FIG. 3, the expression of GFP was significantly increased in CD8⁺ FLAG (CAR)⁺ T cells isolated from tumors relative to the equivalent cell populations isolated from spleens when GFP was expressed under the control of the endogenous regulatory elements of RGS16, with a GFP MFI of 9200 (Day 7) or 12312 (Day 14) in the tumor relative to a GFP MFI of 283 (Day 7) or 588 (Day 14) in the spleen (FIG. 4). The proliferation and phenotypes of the CRISPR-HDR edited CAR T cells were consistent with controls (FIGS. 5 and 6), indicating that incorporation of the HDR template in-frame of RGS16 did not adversely impact CAR T cell function in vivo.

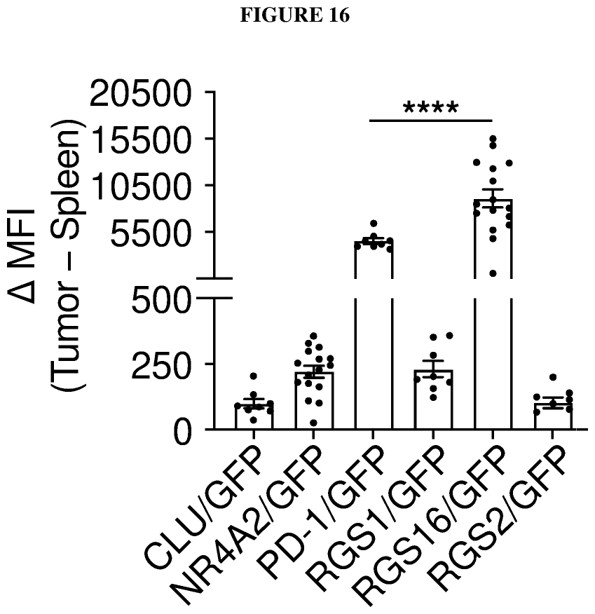

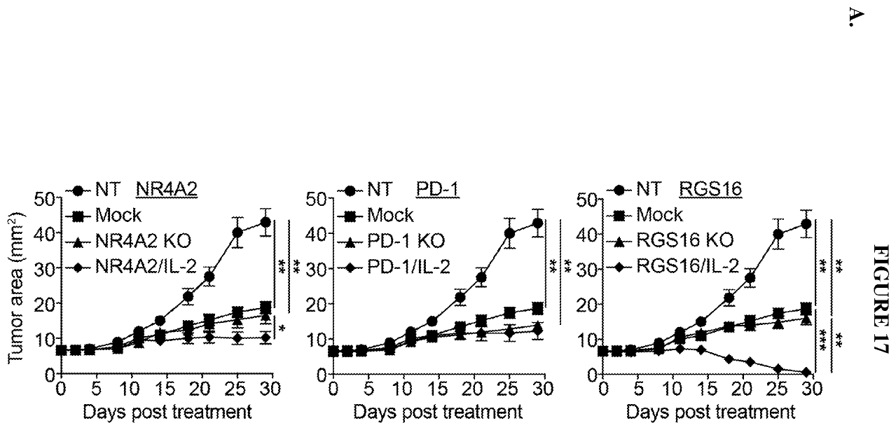

Notably, CLU/, NR4A2/and RGS2/GFP CAR T cells exhibited tumor-restricted expression, with <10% of CAR T cells expressing GFP in the spleen. Of these three promoters, NR4A2 supported the highest GFP expression in the tumor. On the other hand, whilst RGS16/GFP CAR T cells demonstrated similar GFP expression levels in the spleen compared to PD-1/GFP CAR T cells, it supported potent intratumoral GFP intensity at levels that were significantly higher than those supported by PDCD1 (FIG. 16). These data indicate that the RGS16 promoter is ideal for transgenes that require potent intratumoral expression to mediate their anti-tumor effects.

To further confirm the tumor specificity of the RGS16 promoter, GFP expression of RGS16/GFP CAR T cells in the liver was also assessed, as it would be crucial to minimize the expression of pro-inflammatory factors in this tissue. RGS16/GFP CAR T cells exhibited similar levels of GFP in the liver compared to the spleen, further supporting the utility of this promoter to drive tumor-directed transgene expression (Tables 7 and 8).

Murine OT-I T cells engineered to express GFP via the NR4A2, PD-1 or RGS16 gene locus were also injected into mice bearing AT3 ova tumors. At 8 days post-adoptive transfer, it was shown that GFP was highly expressed by intratumoral RGS16/GFP OT-I T cells relative to NR4A2/GFP OT-I cells, with lower GFP expression in the liver and spleen relative to PD-1/GFP OT-I T cells (Table 9; FIG. 21).

### Example 5—Assessment of RGS16 HDR Templates

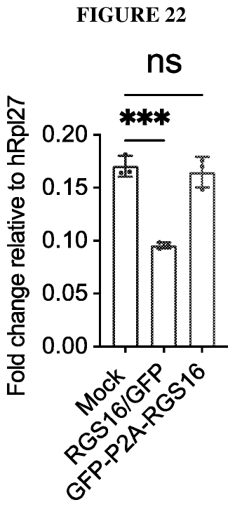

Maintenance of the expression of the endogenous RGS16 gene was achieved by incorporating a P2A linker in the HDR template. The expression of RGS16 was validated by qRT-PCR (primer sequences shown in Table 4), demonstrating that the editing strategy using the GFP-P2A-RGS16 HDR template maintains endogenous RGS16 expression (FIG. 22).

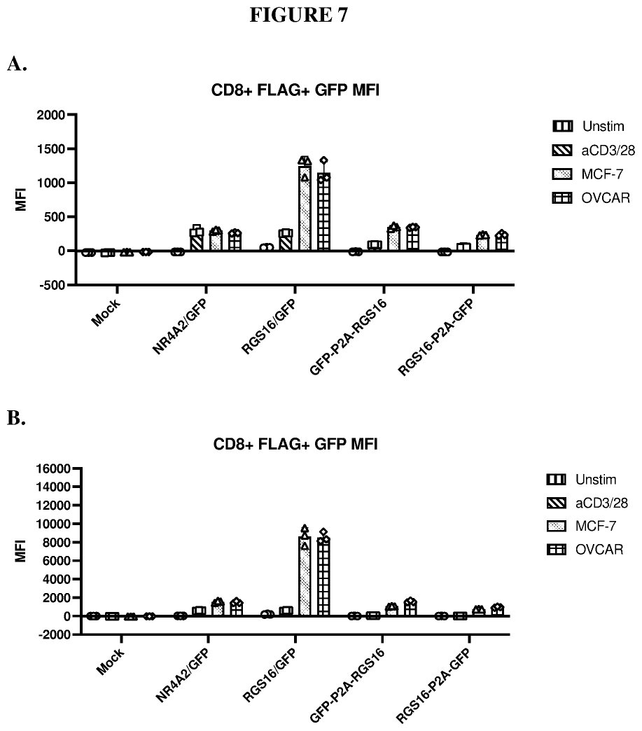

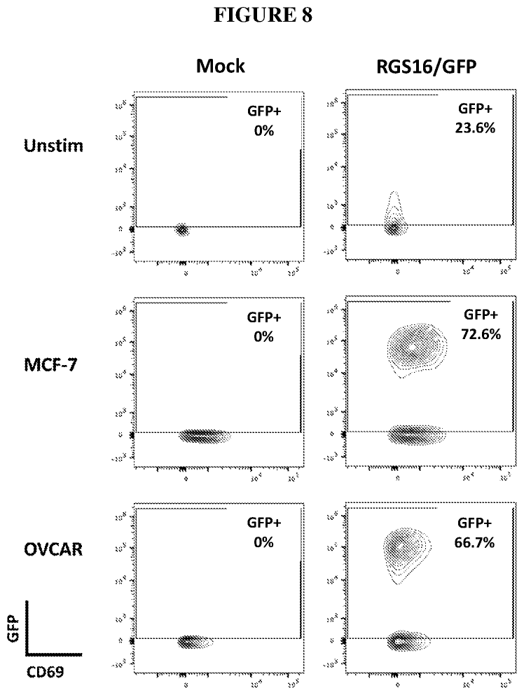

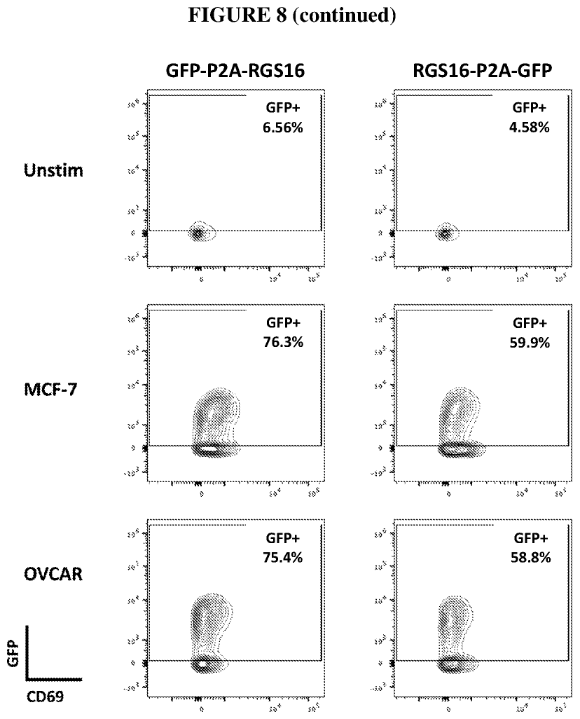

Cells with maintained endogenous RGS16 expression (e.g., GFP-P2A-RGS16 and RGS16-P2A-GFP) had a relatively high proportion of GFP-positive cells following stimulation with plate-bound anti-CD3+anti-CD28, MCF-7 or OVCAR tumour lines, but with low levels of GFP expression when unstimulated (FIGS. 7 and 8). In contrast, cells with disrupted endogenous RGS16 expression (i.e., RGS16/GFP) showed a higher proportion of GFP positive cells when unstimulated (FIGS. 7 and 8).

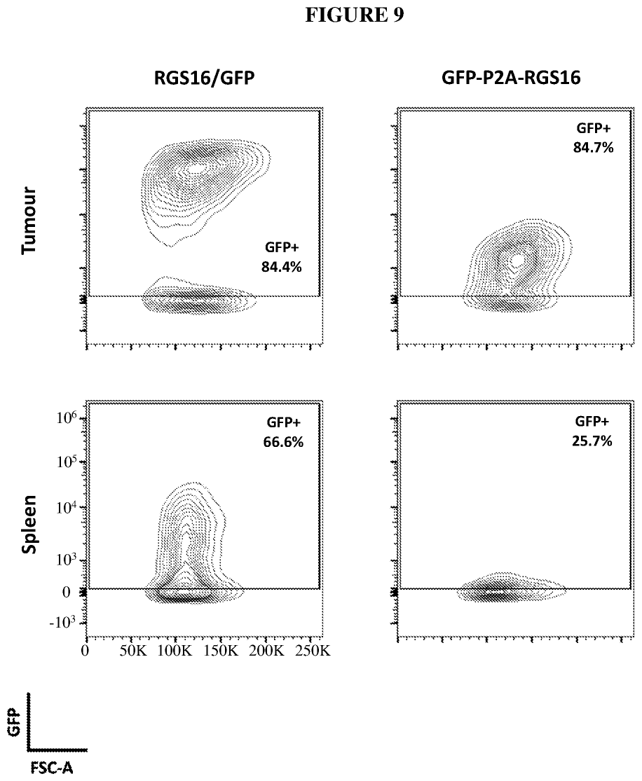

As shown in FIG. 9, maintenance of endogenous RGS16 expression by incorporation of a P2A linker in the HDR template (GFP-P2A-RGS16) maintained high levels of GFP expression in the tumor in vivo, while reducing the background expression of GFP in the spleen and liver when compared to the HDR template that leads to disruption of endogenous RGS16 expression (RGS16/GFP) (Table 8).

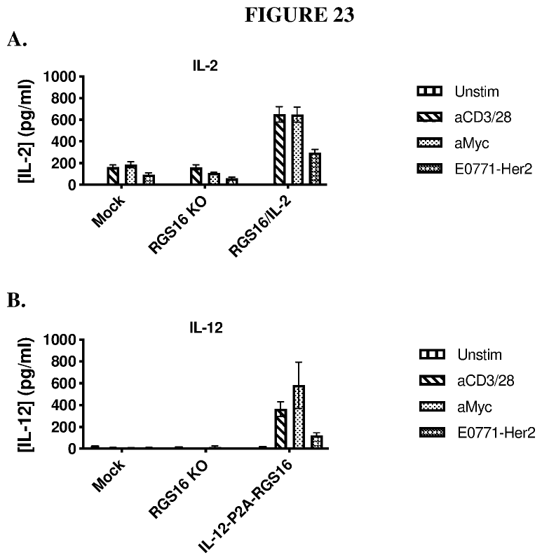

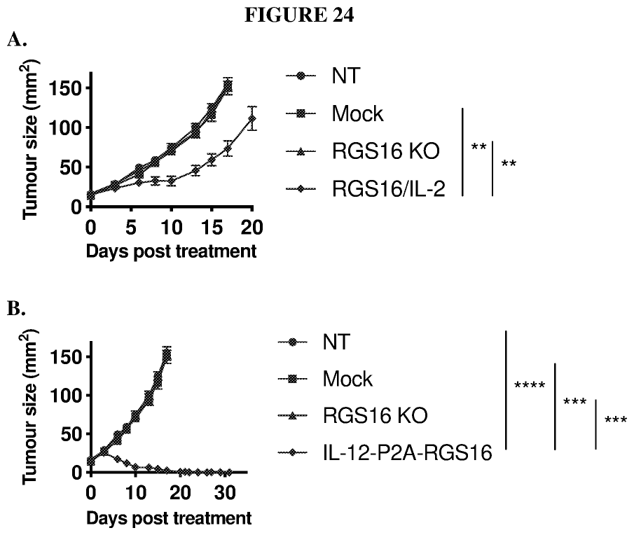

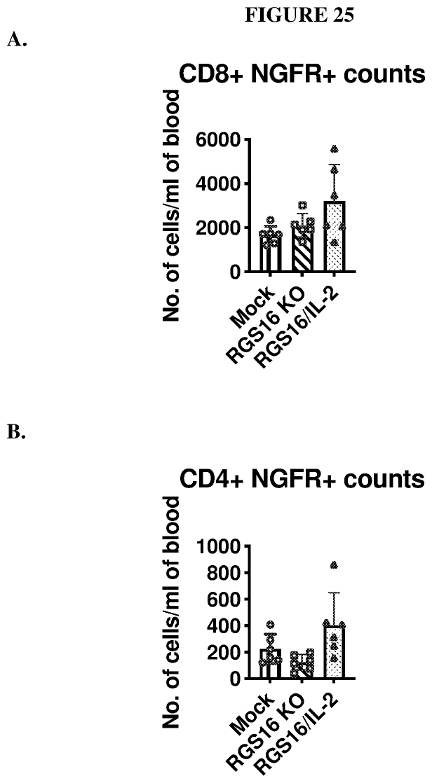

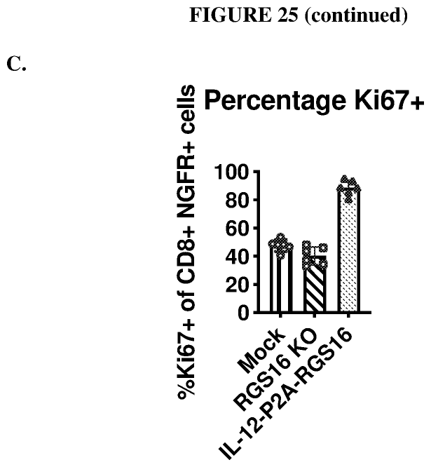

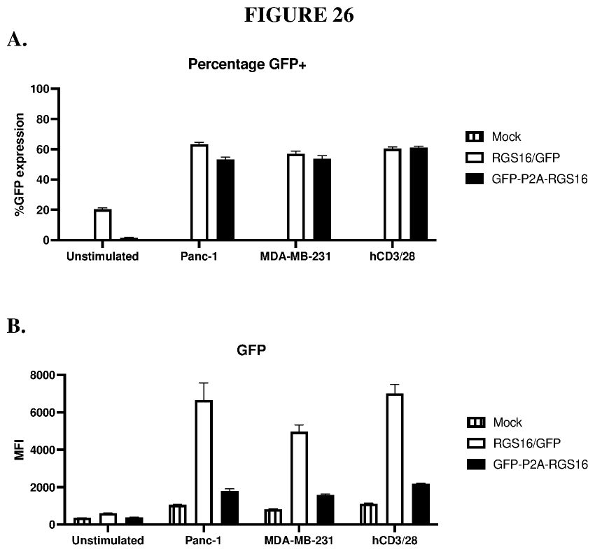

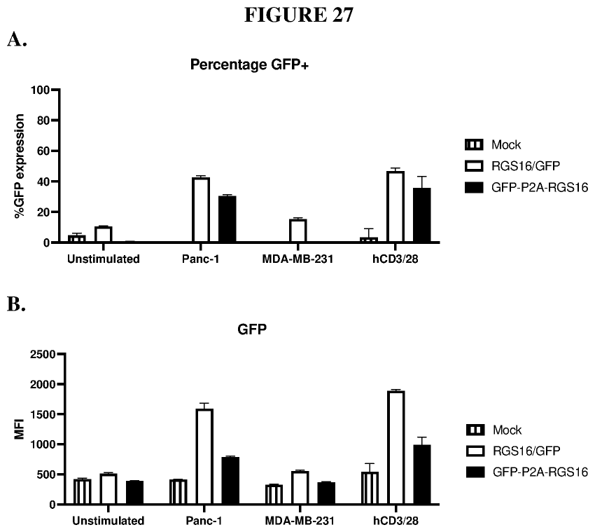

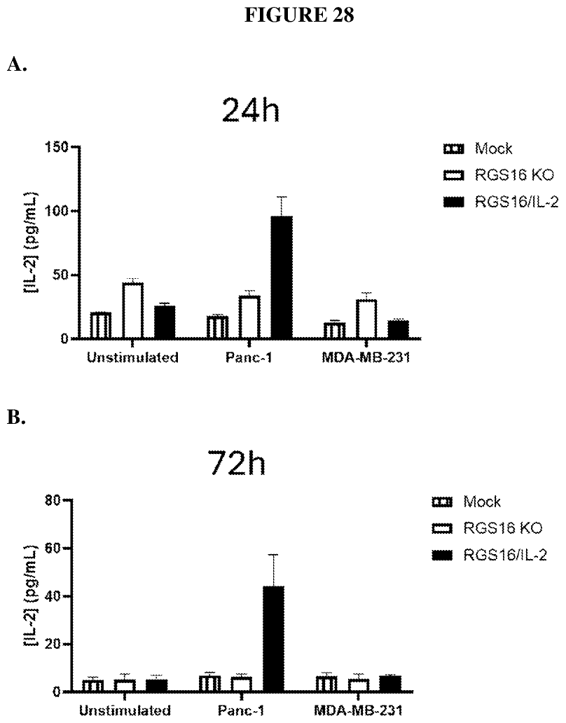

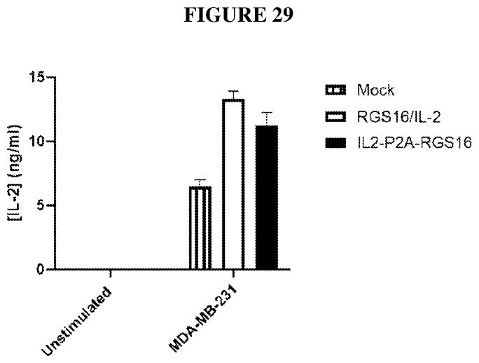

RGS16 HDR templates were further validated for IL-2 and IL-12 expression in vitro and in vivo by engineering murine anti-Her2 CAR T cells to express IL-2 and IL-12 via the RGS16 target locus, where endogenous RGS16 expression was either disrupted (RGS16/IL-2) or maintained (IL-12-P2A-RGS16). For the in vitro analysis, CAR T cells were stimulated with plate-bound anti-CD3/28, anti-Myc or E0771-Her2 tumor cells for 24 hours before IL-2 or IL-12 secretion was assessed by cytometric bead array (FIG. 23). The in vivo results are presented in FIGS. 24 and 25, following injection of the murine anti-Her2 CAR T cells into E0771-Her2 tumor-bearing mice.

### Example 6—Impact of Endogenous Gene Knockout

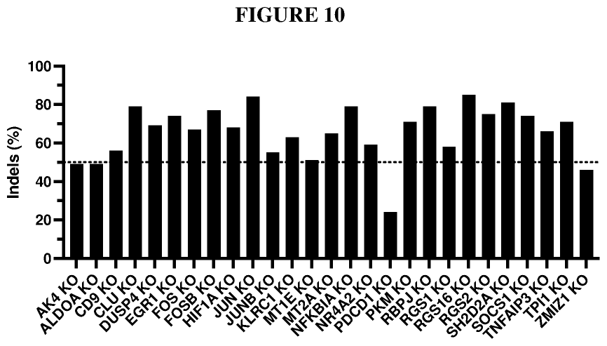

The impact of secondary endogenous gene knockout was assessed in vitro using CRISPR-edited human anti-Lewis Y CAR T cells comprising a genetic modification that disrupts the expression, function or both expression and function of B2M, AK4, ALDOA, CD9, CLU, DUSP4, EGR1, FOS, FOSB, HIF1A, JUN, JUNB, KLRC1, MTIE, MT2A, NFKBIA, NR4A2, PDCD1, PKM, SOCS1, RBPJ, RGS1, RGS2, SH2D2A, TNFAIP3, TPI1, ZMZ1 (FIG. 10).

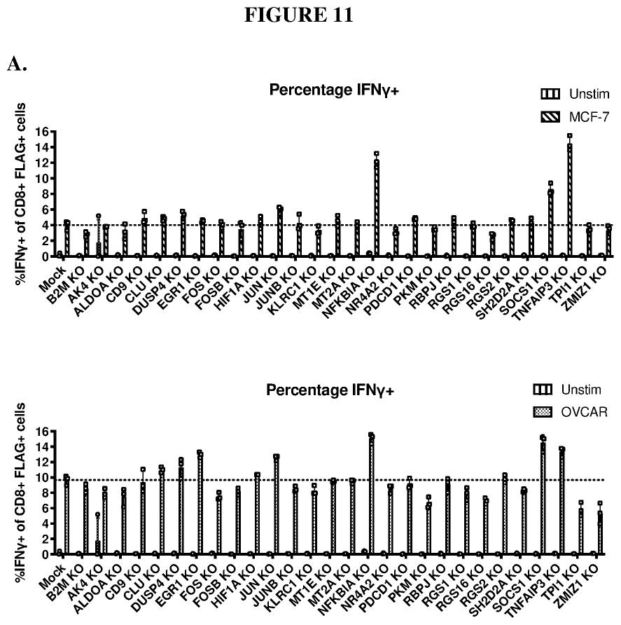

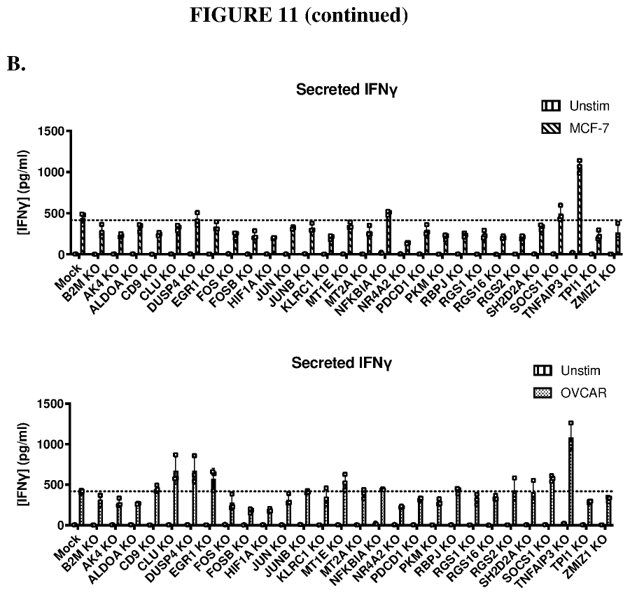

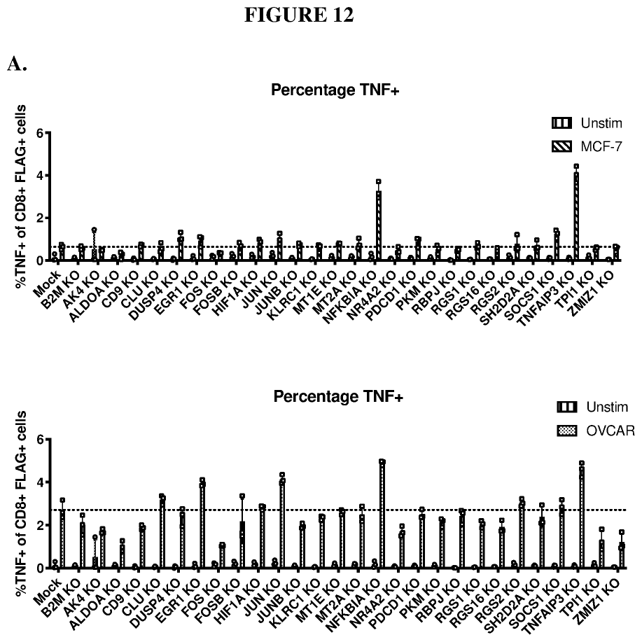

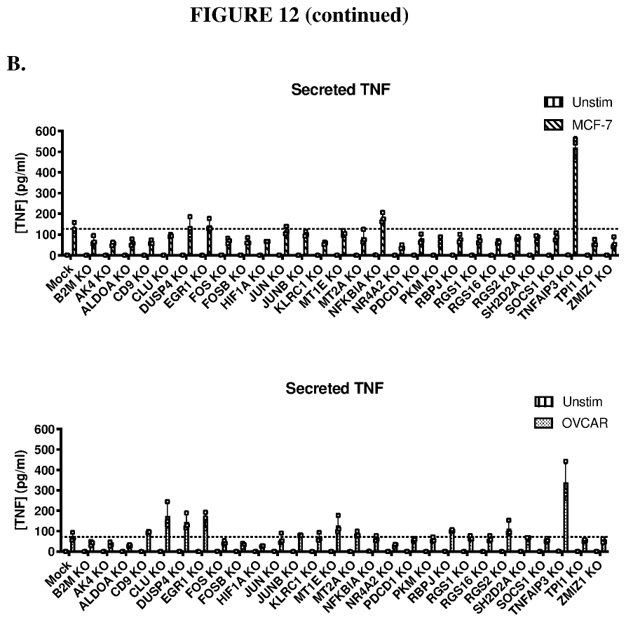

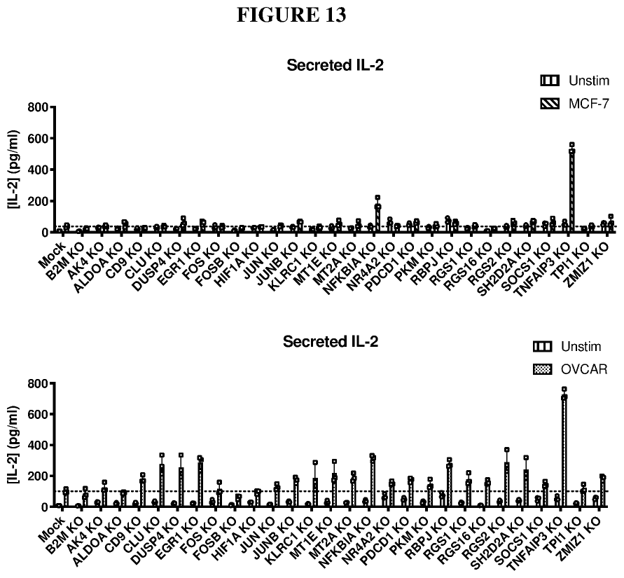

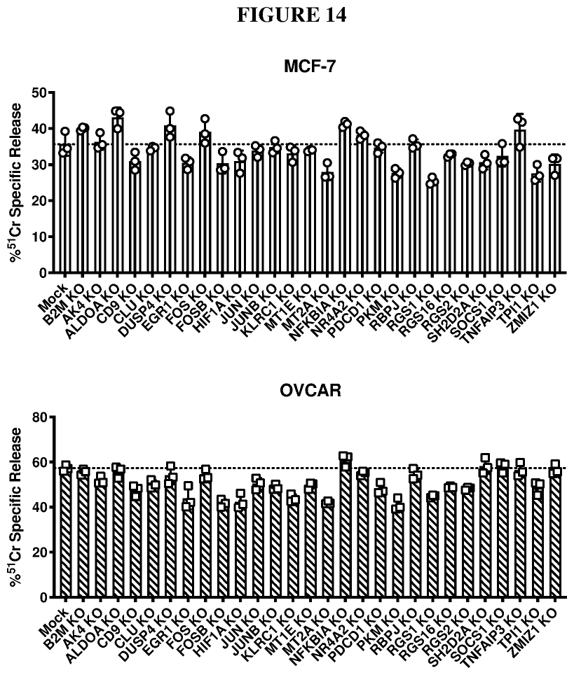

Cytokine production and cytotoxicity was assessed, which demonstrated that a number of secondary endogenous genes, including NFKBIA, SOCS1, and TNFAIP3, may contribute to the anti-tumor immune response associated with immunotherapy when knocked out, e.g., by enhancing cytokine production by CAR T cells (FIGS. 11 to 13) and cytotoxicity (FIG. 14).

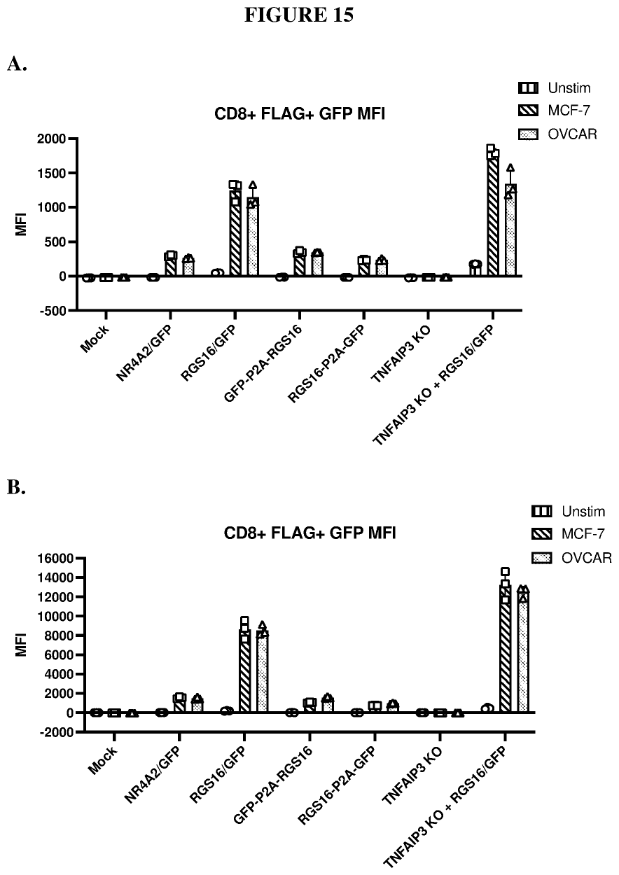

When combined with the insertion of a HDR template in-frame of RGS16, it was shown that knockout of TNFAIP3 improved the level of GFP expression in human anti-Lewis Y CAR T cells stimulated with either MCF-7 or OVCAR cells (FIG. 15).

### Example 7—Therapeutic Efficacy of Human CRISPR-Engineered CAR T Cells

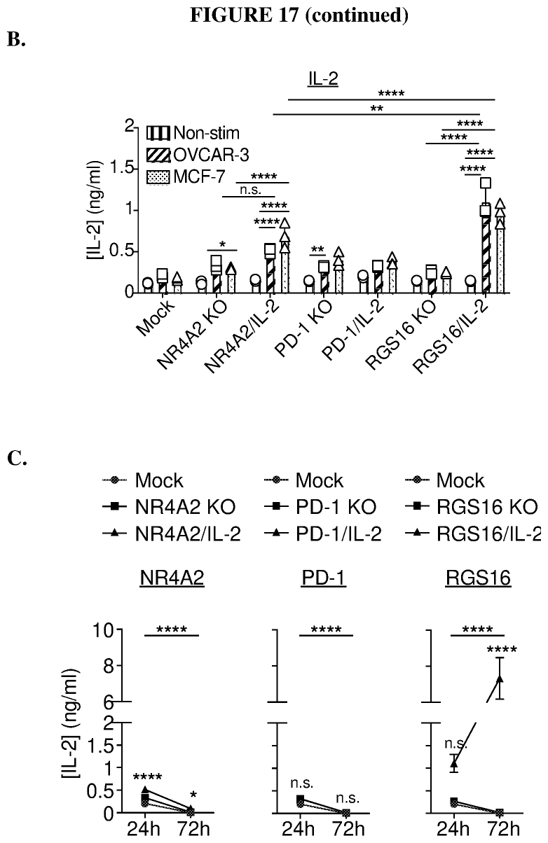

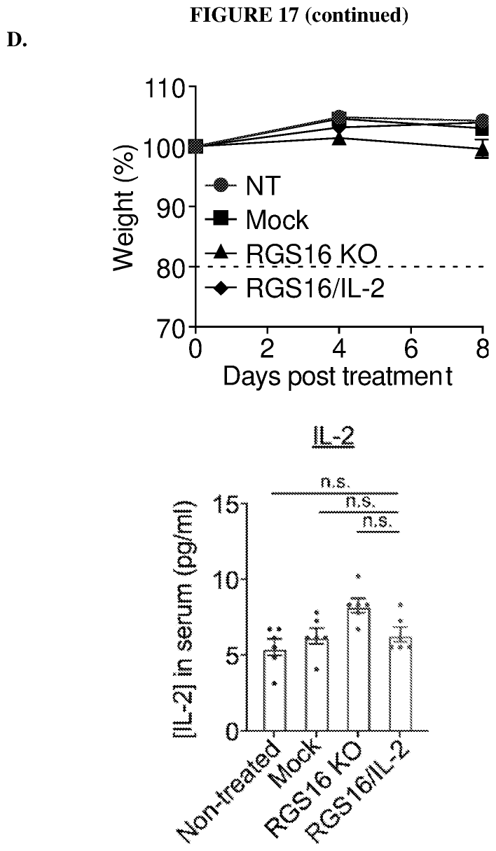

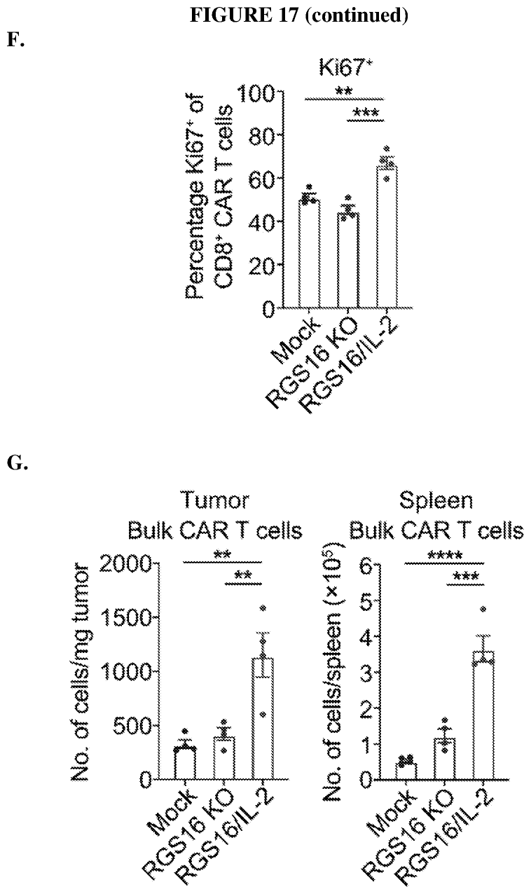

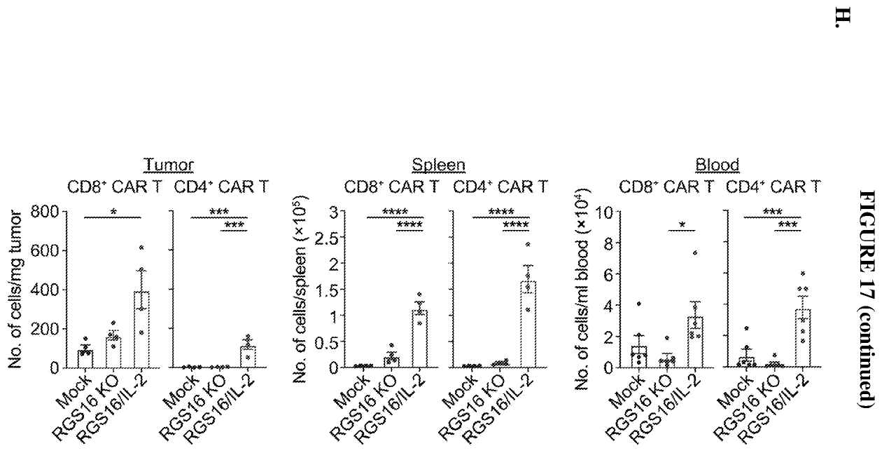

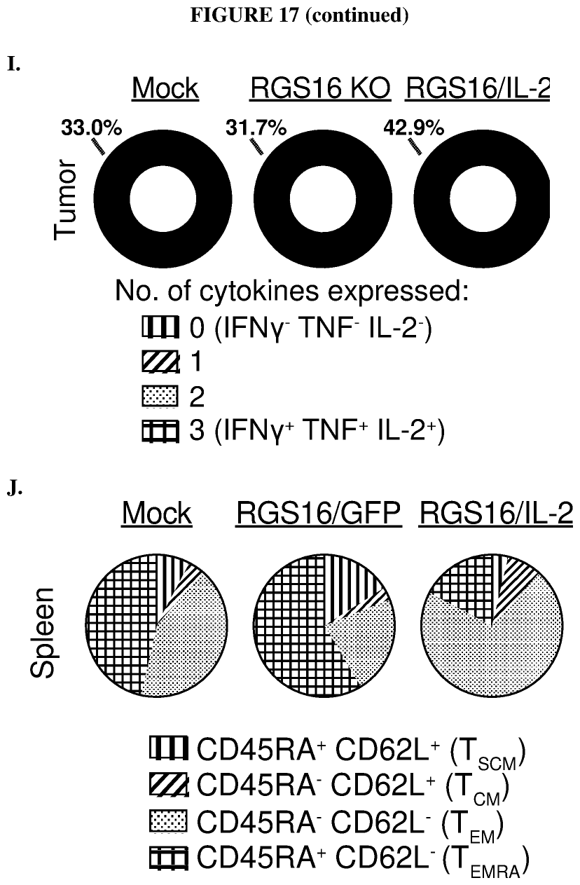

As RGS16 was previously identified as a promoter with potent intratumoral expression (FIG. 16), the therapeutic effect of RGS16/IL-2 human CAR T cells was compared to that of NR4A2/IL-2 and PD-1/IL-2 human CAR T cells. As shown in FIG. 17A, RGS16/IL-2 CAR T cells exhibited a superior anti-tumor effect compared to NR4A2/IL-2 and PD-1/IL-2 CAR T cells. Moreover, RGS16/IL-2 human CAR T cells exhibited significantly higher IL-2 expression than PD-1/or NR4A2/IL-2 engineered CAR T cells upon in vitro stimulation with OVCAR-3 or MCF-7 tumor cells (FIG. 17B-C). This was in the absence of toxicities, indicated by stable body weight and no differences in serum IL-2 levels compared to the control groups (FIGS. 17D-E), suggesting that the RGS16 promoter is suitable for driving intratumoral expression of factors with a milder toxicity profile. Mechanistically, RGS16/IL-2 CAR T cells exhibited significantly increased Ki67 expression (FIG. 17F), concurrent with significantly enhanced numbers of both CD8⁺ and CD4⁺ T cells within the tumor, spleen and blood (FIGS. 17G and H), as well as improved polyfunctionality, indicated by an increased proportion of T cells that co-expressed IFNγ and TNF (FIG. 171). Furthermore, RGS16/IL-2 CAR T cells exhibited an increased frequency of the CD45RA CD62L; effector memory-like subset in the spleen (FIG. 17J).

### Example 8—Clinical Applicability of CRISPR-Engineered Armoured CAR T Cells

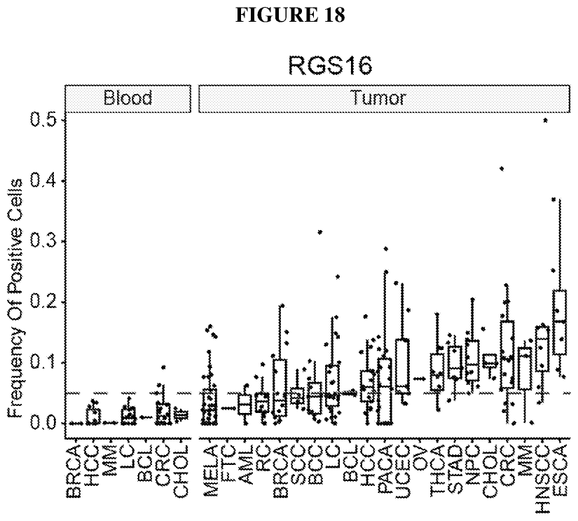

To interrogate the transcriptional activity of the RGS16 promoter in patient T cells, publicly available scRNA-seq data was analysed for RGS16 expression in T cells from the blood and tumor site of patients from a broad range of cancer types (Zheng et al., 2021, Science, 374). This revealed a higher frequency of RGS16-expressing T cells within the tumor compared to peripheral blood (FIG. 18), suggesting that these genes are likely to support tumor-localized transgene expression across numerous different tumor types when engineered through the gene editing method described herein.

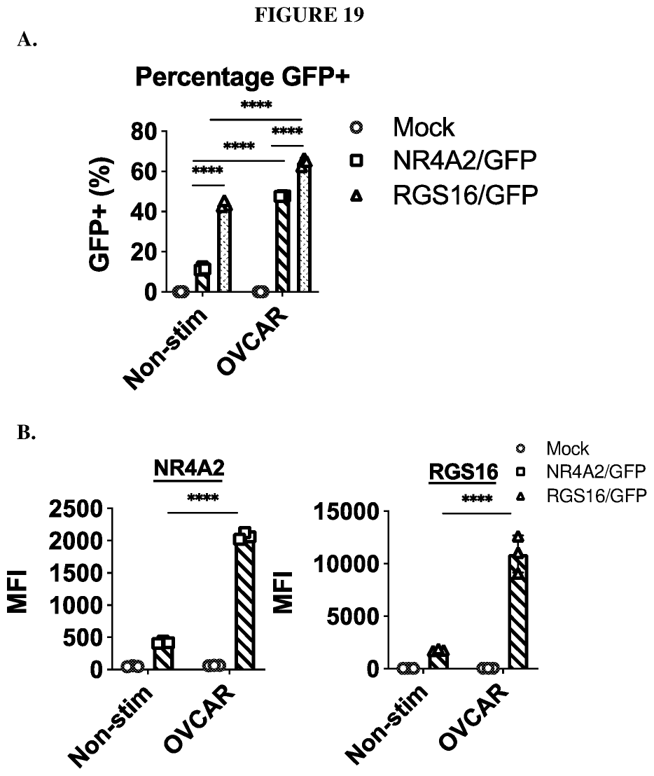

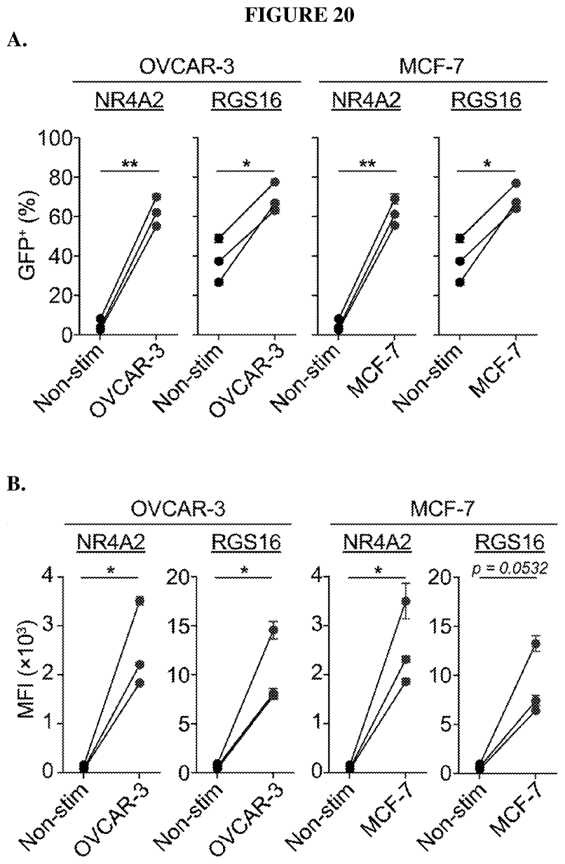

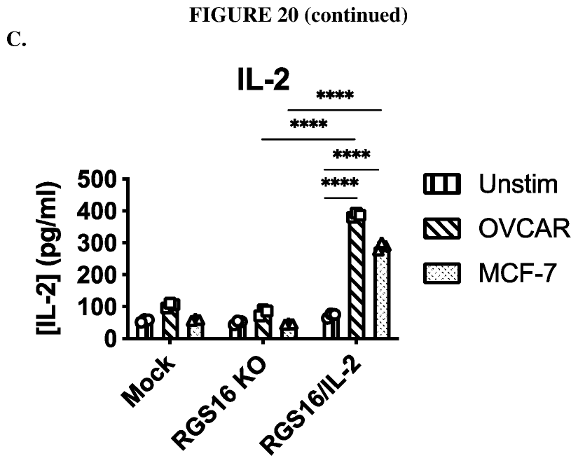

Additionally, it was investigated whether patient-derived T cells could be reprogrammed via the gene editing method and systems described herein to secrete transgenes upon stimulation in vitro. T cells from a patient with multiple myeloma (MM) were first engineered to express an anti-Lewis Y CAR as well as GFP via the RGS16 promoter. Upon in vitro stimulation with OVCAR-3 tumor cells, similar results as our findings in healthy donors were observed, with potent GFP expression in RGS16/GFP CAR T cells (FIG. 19). This was further demonstrated in T cells isolated from 3 diffuse large B cell lymphoma (DLBCL) patients (FIG. 20). Furthermore, generation of RGS16/IL-2 anti-Lewis Y CAR T cells from one of the DLBCL patients led to a significant increase in IL-2 production upon stimulation with OVCAR-3 or MCF-7 tumor cells in vitro (FIG. 20C).

Taken together, these data demonstrate the clinical potential of the gene editing method and systems described herein to engineer armoured CAR T cells, where the endogenous regulatory machinery of genes with tumor-restricted expression, e.g., RGS16, are leveraged to drive tumor-localised expression of immunomodulatory factors.

### DISCUSSION

Collectively, these data are enabling of engineered T cells that express heterologous nucleic acid sequences (such as those encoding immunomodulatory factors) in a tumor-site selective manner, and drive potent intratumoral transgene expression. Unlike previous approaches to the generation of “armored” T cells for use in immunotherapy, the engineered T cells of the present disclosure, by virtue of providing tumor-site selective expression of the heterologous nucleotide sequence, advantageously minimise or otherwise avoid the unwanted off-target effects associated with the constitutive expression of heterologous nucleotide sequences (e.g., toxicity) that has been observed in previous studies.

It has surprisingly been shown that the introduction of a heterologous nucleotide sequence in-frame of RGS16, RGS1, RGS2 or CLU under the control of an endogenous regulatory element, wherein expression of the heterologous nucleotide sequence is controlled by the endogenous regulatory elements of RGS16, RGS1, RGS2 or CLU, enables the tumor-selective expression of immunomodulatory factor(s) encoded by the heterologous nucleotide sequence. Moreover, the present inventors have demonstrated that undesired expression of the heterologous nucleotide sequence in non-tumor tissues can be reduced by adjusting the components and conformation of the heterologous nucleotide sequence or HDR templates. The reduction in off-target expression of the heterologous nucleotide sequence in the engineered T cells (e.g., within the spleen) observed when using HDR templates comprising a linker that helps retain endogenous target gene expression indicates that the engineered T cells described herein may have a superior safety and therapeutic profile relative to previous approaches to generate “armored” T cells, demonstrating that the engineered T cells, gene editing systems, and methods disclosed herein are particularly useful for clinical applications.

It has been demonstrated that coupling transgene expression to endogenous tumor-inducible genes enables the highly tumor-localized production of immunomodulatory factors. In contrast to synthetic inducible systems, the methods and systems disclosed herein encapsulate not only the promoter, but all regulatory elements of a tumor-specific gene, which enables the leveraging of in-built circuits to achieve spatiotemporal control of transgene expression. Through in vitro and in vivo assessment of their capacity to regulate GFP expression, RGS16 supported the highest intratumoral transgene expression. Additionally, by expressing transgenes via the RGS16 promoter while retaining expression of endogenous RGS16, this was demonstrated to greatly enhance the tumor-specificity of transgene expression, enabling potent intratumoral delivery of transgenes while maintaining minimal peripheral expression. Moreover, the clinical utility of the engineered T cells disclosed herein has been demonstrated using patient-derived T cells, where endogenous RGS16 regulatory elements_supported potent transgene expression that is comparable to what has been observed with edited healthy donor T cells upon in vitro CAR stimulation. Furthermore, analysis of RGS16 expression in patient datasets revealed increased expression of RGS16 in intratumoral T cells across a broad range of cancer types compared to several matched peripheral blood datasets.

TABLE 1
Exemplary sgRNAs

| Target | sgRNA | SEQ ID NO |
|---|---|---|
| Human RGS16 | CGCAGCCAUGUGCCGCACCC | 1 |
| Human RGS16 | CCTGAGTCTCCACGGCAGTG | 2 |
| Human RGS16 | CAAAGAGUUCAAGACACGUC | 3 |
| Human RGS1 | CAUUUUGUCUAACUUUGGAG | 4 |
| Human RGS1 | UCAUUCCAGAUUCCAGAUGU | 5 |
| Human RGS2 | CGCCCAGCCGCAAACAGCCG | 6 |
| Human RGS2 | AGGCAGUGGCCACAAGAGCG | 7 |
| Human CLU | AGACUCUGCUGCUGUUUGUG | 8 |
| Human CLU | UCUUGCAGAAAUGUCCAAUC | 9 |
| Human PD-1 | UCCAGGCAUGCAGAUCCCAC | 10 |
| Human TNFAIP3 | GGCUGAACAAGUCCUUCCUC | 11 |
| Human NFKBIA | CAUGGAUGAUGGCCAAGUGC | 12 |
| Human NR4A2 | GCCUGAACACAAGGCAUGGC | 13 |
| Human NR4A2 | UCCGGCGACGCUUGUCCACU | 14 |
| Human SOCS1 | UAGGAUGGUAGCACACAACC | 15 |
| Murine RGS16 | GGACGCAGGACAGCACGUCG | 38 |

TABLE 2
Exemplary HDR templates

| HDR template | SEQ ID NO |
|---|---|
| Human RGS16/GFP | 16 |
| Human GFP-P2A-RGS16 | 17 |
| Human RGS16-P2A-GFP | 18 |
| Human RGS1/GFP | 19 |
| Human RGS2/GFP | 20 |
| Human CLU/GFP | 21 |
| Human PD-1/GFP | 22 |
| Human NR4A2/GFP | 23 |
| Human TNFAIP3/GFP | 24 |
| Human RGS16/IL-2 | 39 |
| Human RGS16/IL-12 | 40 |
| Human IL-2-P2A-RGS16 | 41 |
| Human IL-12-P2A-RGS16 | 42 |
| Murine RGS16/IL-2 | 43 |
| Murine RGS16/IL-12 | 44 |
| Murine IL-2-P2A-RGS16 | 45 |
| Murine IL-12-P2A-RGS16 | 46 |
| Murine RGS16/GFP | 47 |
| Murine GFP-P2A-RGS16 | 48 |

TABLE 3
Exemplary heterologous nucleotide sequences
encoding immunomodulatory factors

| Immunomodulatory factor | SEQ ID NO |
|---|---|
| Human NGFR | 25 |
| Human CXCL9 | 26 |
| Human CXCL10 | 27 |
| Human CXCL9-P2A-CXCL10 | 28 |
| Human TNF | 29 |
| Human IFNg | 30 |
| Human TNF-P2A-IFNg | 31 |
| Human IL-2 | 32 |
| Human IL-12 | 33 |
| Human IL-12 | 34 |
| Murine IL-2 | 49 |
| Murine IL-12 | 50 |

TABLE 4
RT-qPCR primer sequences

| | | | SEQ ID |
|---|---|---|---|
| Primer | Direction | Sequence (5′ - 3′) | NO: |
| Human RGS16 | Forward | GGGCTGCGATACTGGGAG | 51 |
| Human RGS16 | Reverse | TTCAGGAAAGCGTGGAAGG | 52 |

TABLE 5
GFP expression by human anti-Lewis Y CAR T cells
edited to express Gfp at the RGS16, PD-1 or NR4A2
gene loci following co-culture with Lewis Y expressing
OVCAR-3 or MCF-7 tumor cells for 72 hours

| | GFP expression (%) | | |
|---|---|---|---|
| | | | OVCAR-3 |
| | Non-stimulated | MCF-7 stimulated | stimulated |
| Mock | 0 | 0 | 0 |
| PD-1/GFP | 20.5 | 68.5 | 62.5 |
| NR4A2/GFP | 11.2 | 68.3 | 67.4 |
| RGS16/GFP | 53.7 | 76.3 | 72.4 |
| GFP-P2A-RGS16 | 5.24 | 84.1 | 82.1 |

TABLE 6
GFP expression by murine OT-1 T cells edited to express Gfp at
the RGS16, PD-1 or NR4A2 gene loci following stimulation with
plate-bound anti-CD3/28 or AT3 ova tumor cells for 24 hours

GFP expression (%)

| | | anti-CD3/28 | AT3 ova |
|---|---|---|---|
| | Non-stimulated | stimulated | stimulated |
| Mock | 0.54 | 0.37 | 0.51 |
| PD-1/GFP | 28.7 | 41.3 | 37.4 |
| NR4A2/GFP | 3.09 | 25.8 | 24.3 |
| RGS16/GFP | 19.0 | 39.2 | 37.8 |
| GFP-P2A-RGS16 | 1.24 | 36.1 | 31.7 |

TABLE 7
GFP expression in tumor or spleen following adoptive
transfer of human anti-Lewis Y CAR T cells edited
to express Gfp at the RGS16, RGS1, RGS2 and CLU gene
loci to mice bearing subcutaneous OVCAR-3 tumors

GFP expression (%)

| | Tumor | Spleen |
|---|---|---|
| Mock | 0 | 0 |
| CLU/GFP | 22.5 | 0.7 |
| RGS1/GFP | 48.0 | 31.6 |
| RGS2/GFP | 18.2 | 3.4 |
| RGS16/GFP | 79.2 | 26.5 |

TABLE 8
GFP expression in liver, spleen or tumor following adoptive
transfer of human anti-Lewis Y CAR T cells edited to
express Gfp at the RGS16, PD-1 or NR4A2 gene loci to
mice bearing subcutaneous OVCAR-3 tumors

| | GFP expression (%) | | |
|---|---|---|---|
| | Liver | Spleen | Tumor |
| Mock | 0.5 | 1.5 | 0 |
| PD-1/GFP | 43 | 32.1 | 80.6 |
| NR4A2/GFP | 6.6 | 6.6 | 30.0 |
| RGS16/GFP | 33.6 | 40.5 | 75.8 |
| GFP-P2A-RGS16 | 4.8 | 3.3 | 75.7 |

TABLE 9
GFP expression in liver, spleen or tumor following adoptive transfer
of murine OT-I T cells edited to express Gfp at the RGS16, PD-1
or NR4A2 gene loci to mice bearing AT3-ova tumors

| | GFP expression (%) | | |
|---|---|---|---|
| | Liver | Spleen | Tumor |
| Mock | 0.1 | 0 | 0.6 |
| PD-1/GFP | 25.1 | 19.2 | 32.4 |
| NR4A2/GFP | 1.1 | 0.9 | 22 |
| RGS16/GFP | 7.6 | 5.3 | 28.9 |
| GFP-P2A-RGS16 | 0.9 | 0.5 | 31.8 |

1. An engineered T cell comprising a heterologous nucleotide sequence encoding at least one immunomodulatory factor, wherein the heterologous nucleotide sequence is introduced in-frame of an endogenous gene of the T cell, wherein expression of the endogenous gene is under the control of endogenous regulatory elements, wherein expression of the heterologous nucleotide sequence is controlled by the endogenous regulatory elements of the endogenous gene, and wherein the endogenous gene is selected from the group consisting of RGS16, RGS1, RGS2, and CLU.

2-37. (canceled)
